(19) United States
(12) Patent Application Publication
(10) Pub. No.: US 2021/0205462 A1
(43) Pub. Date: Jul. 8, 2021

(54) Translocation into Eukaryotic Cells

(71) Applicant: The University Court of the University of Aberdeen, Aberdeen (GB)

(72) Inventors: Franziska Trusch, Aberdeen (GB); Pieter Van West, United Kingdom (GB)

(73) Assignee: The University Court of the University of Aberdeen, Aberdeen (GB)

(21) Appl. No.: 17/056,545

(22) PCT Filed: May 29, 2019
(86) PCT No.: PCT/EP2019/063993

§ 371 Date: Nov. 18, 2020

(30) Foreign Application Priority Data
May 31, 2018 (GB) 1808910.2

(51) Int. Cl. A61K 47/64; A61K 47/69; A61K 39/385; C12N 15/88

(52) U.S. Cl. CPC A61K 47/64; A61K 47/6923; A61K 2039/6031; C12N 15/88; A61K 47/6925; A61K 39/385

(57) ABSTRACT

The present disclosure relates to compositions comprising an SpHtp1 peptide translocation sequence and uses of such compositions. Also described are compositions comprising a payload coupled to an SpHtp1 peptide translocation sequence and their uses.

### EARLIER APPLICATION

The present application claims priority from United Kingdom application GB1808910.2 filed on 31 May 2018.

### FIELD OF THE DISCLOSURE

The present disclosure relates to compositions comprising an SpHtp1 peptide translocation sequence and uses of such compositions. Also described are compositions comprising a payload coupled to an SpHtp1 peptide translocation sequence and their uses.

### BACKGROUND

Drug Delivery

Drug therapies can be delivered to patients by a variety of administration routes, each having its own advantages and disadvantages. Not all administration routes can be used for every drug therapy; developments in drug delivery systems thus have the potential to enhance the use of or provide alternative routes for administration of existing therapies, and open the possibility of delivering drugs previously restricted in their application to subjects in need thereof.

Inhalational drug delivery is an attractive route of drug administration as it is non-invasive and allows therapeutic agents to be absorbed quickly and act both locally and systemically while avoiding hepatic first pass metabolism. However, this route of administration has its own set of challenges, such as suitable formulation of the drug to ensure absorption in the lungs.

Despite advances in this field there remains an unmet need for further drug delivery systems.

Respiratory Disease

Respiratory diseases such as asthma and chronic obstructive pulmonary disease (COPD) are among the most widespread in the developed world. Inhalational drug delivery is an attractive, non-invasive, means of treating a wide range of lung diseases, but presents the additional challenge of ensuring local delivery of drug therapies while minimising absorption into the circulatory system (and thus undesirable delivery to other areas of the body). There is an ongoing need for drug delivery systems for effective treatment of respiratory diseases.

Vaccines

Vaccines against a broad range of pathogenic antigens are available in the form of live attenuated vaccines, polypeptide-based vaccines, polysaccharides, conjugated polysaccharides, and naked DNA vaccines among others. Conventionally, vaccines are prepared as a reconstituted liquid and administered to a patient by injection. This has a number of disadvantages, including the use of needles and the associated risks of contamination and transmission of disease.

Accordingly, there is an ongoing need for improved vaccine delivery systems, particularly for human and mammalian use.

Effector Proteins and Payload Delivery

Several prokaryotic and eukaryotic microbial pathogens have evolved mechanisms for delivery of pathogenicity effector proteins into host cells. Effector proteins modulate molecular processes in the host cells to suppress immune responses, thereby helping to establish an infection.

SpHtp1, a putative effector from the fish pathogenic oomycete, Saprolegnia parasitica, has been shown to be delivered by the pathogen into cells of a rainbow trout cell line (RTG-2) in the absence of the pathogen. Subsequently, it was demonstrated that this effect relied only on the helical integrity of a 44-amino acid stretch out of 198 amino acids for the full-length protein (amino acids 24-68; SpHtp1²⁴⁻⁶⁸), and that payloads coupled to this peptide fragment could be effectively delivered across the lipid membrane of eukaryotic cells, both in vitro and in live fish (WO 2011/148135; WO 2014/191759). When coupled to an antigenic payload, this delivery into live cells was effective enough to elicit an immune (antibody) response against the antigen.

This effect has been demonstrated for small molecule and peptide payloads with fish cells; experiments demonstrated no translocation into animal cells (Wawra et al. 2012, PNAS Vol 109 (6) pp 2096-2101; Wawra et al. 2012, MPMI Vol 26 (5) pp 528-36; WO 2014/191759).

The present disclosure has been devised in light of the above considerations and addresses these and other needs.

### SUMMARY OF THE DISCLOSURE

The present authors have unexpectedly found that the short fragment of the SpHtp1 RxLR effector protein SpHtp1²⁴⁻⁶⁸ can be used to enhance the translocation of particles in the μm size range across the lipid membrane of eukaryotic cells. Thus, according to one aspect of the disclosure there is provided a composition comprising: a particle; and, a translocation sequence comprising a polypeptide having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6 coupled to the particle.

In some embodiments the particle is at least 10, 20, 30, 40, 50, 60, 70, 80, 90 or 100 nm in size. Such particles are a number of orders of magnitude larger in size (and even larger again in volume) than the peptide and small molecule payloads previously shown to be translocated into cells by effector protein-derived translocation sequences.

Particles which can be translocated into cells by this sequence are not limited to those formed of biological material. Accordingly, in some embodiments, the particle can be: a metallic, semiconductor, or fluorescent particle; a magnetic particle; a polymeric or liposomal nanoparticle; a particle having an outer membrane comprising lipid, plastic, or other synthetic or natural polymer; a virus or viral particle; or, a bacteria, fungus, or other disease causing microbe.

In some embodiments the particle encapsulates or contains a cargo molecule, which may be: a marker or imaging agent; a polypeptide or nucleic acid; an antibody; an antigen, immunogen, or vaccine; an antibiotic agent; a lipid; a small organic molecule; a metal; a therapeutic agent; a protective agent; or a cytotoxic agent, among others. Thus, the surprising discovery of the present disclosure opens the possibility of delivering any molecule of interest into eukaryotic cells. This will be particularly advantageous in the delivery of drugs and other cargo molecules which are difficult to deliver into the interior of cells.

The translocation sequence can be coupled to the particle by any means of bonding or otherwise associating the particle and the translocation sequence. Thus in some embodiments the translocation sequence is bonded to the particle via a covalent, hydrogen, or electrostatic bond. In other embodiments the translocation sequence is associated with the particle via hydrophobic association or van der Waals interactions. In some embodiments the composition comprises a plurality of translocation sequences coupled to the particle. The number of translocation sequences coupled to the particle is sufficient to enhance translocation of the composition across the membrane of a eukaryotic cell.

In another aspect of the disclosure there is provided a composition comprising a virus, bacteria, fungus, or other disease causing microbe wherein the virus, bacteria, fungus, or other disease causing microbe expresses on its surface a polypeptide comprising a translocation sequence having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6. In some embodiments the virus, bacteria, fungus, or other disease causing microbe is inactivated.

In another aspect there is provided the use of the compositions of the disclosure in methods of treatment of the human or animal body. As described above, the present disclosure opens the possibility of delivering any molecule of interest, including drug therapies, into eukaryotic cells. It is believed that the present disclosure will be particularly advantageous in allowing translocation of drug therapeutics which are presently difficult to deliver into the interior of cells. Furthermore, previous work had incorrectly indicated that SpHtp1 translocation was fish specific, and that these translocation sequences were not able to direct translocation into mammalian cells. This unexpected finding advantageously expands the subject groups known to be treatable with SpHtp1-coupled payloads.

Previous work also indicated that SpHtp1 binds to tyrosine-O-sulfate, and that sulphated cell surface molecules were involved in SpHtp1 translocation. Here the authors demonstrate that SpHtp1 binds to the beta-2 (β2) adrenoceptor, and that SpHtp1 is able to enter human cells via the human β2-adrenoceptor. The β2-adrenoceptor homologue in fish is highly homologous with the mammalian β2-adrenoceptor, thus it is very likely that the β2-adrenoceptor homologue in fish mediates translocation of SpHtp1 into fish cells. In humans, the β2-adrenoceptor is known to be highly expressed in bronchial smooth muscle and epithelial cells, as well as in cardiac myocytes and vascular smooth muscle cells. The compositions of the present disclosure are therefore particularly well suited to administration by inhalational, injection (particularly intravenous), immersion, and oral routes. Accordingly, in some embodiments the compositions of the disclosure are administered by injection, inhalational, immersion, or oral routes.

In some embodiments the method is a method of treatment of a lung/respiratory disease, for example lung cancer; bacterial, viral, or fungal lung infection; asthma; chronic obstructive pulmonary disease (COPD); or, cystic fibrosis.

In another aspect of the disclosure there is provided a vaccine comprising a composition of the disclosure, wherein the particle comprises or encapsulates a target antigen. In some embodiments the vaccine composition comprises a pharmaceutically acceptable excipient, carrier, buffer or stabilizer.

In another aspect there is provided the use of the vaccines of the disclosure, for inducing an immune response in a human or animal subject. In some embodiments the immune response is the generation of antibodies against the particle or an antigen cargo molecule. In some embodiments the vaccine is administered by injection, inhalational, immersion, or oral routes. As outlined above, the compositions of the disclosure, including vaccine compositions of the disclosure, are particularly well suited to administration by these routes. A significant advantage of the vaccine compositions and methods of the disclosure when the subject is a mammal is that the vaccine compositions can be administered by the inhalational administration route. A significant advantage of the vaccine compositions and methods of the disclosure when the subject is a gilled animal is that the vaccine compositions can be administered without handling the gilled animal (e.g. via immersion or oral routes).

In another aspect of the disclosure there is provided use of a translocation sequence comprising a polypeptide having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6 to enhance translocation of a particle across the membrane of a eukaryotic cell, wherein the translocation sequence is coupled to the particle, the membrane comprises a β-adrenergic receptor or homologue thereof, and the translocation sequence interacts with the β-adrenergic receptor to enhance translocation of the nanoparticle across the membrane.

In a further aspect of the disclosure there is provided a method of translocating a particle across the membrane of a eukaryotic cell, the method comprising: coupling a translocation sequence comprising a polypeptide having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6 to a particle; and, contacting the particle with a eukaryotic cell.

In another aspect of the disclosure there is provided a method of delivering a molecule across the membrane of a eukaryotic cell, the method comprising: formulating a molecule into a particle, such that the particle encapsulates or contains the molecule; coupling the particle to a translocation sequence comprising a polypeptide having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6; and, contacting the particle with a eukaryotic cell.

In some embodiments of the uses and methods of the disclosure the cell is an in vitro or ex vivo cell. The surprising discovery of the present disclosure opens the possibility of delivering any molecule of interest into eukaryotic cells. Advantageously, this includes the delivery of drugs and other cargo molecules, such as nucleic acids or vectors, into the interior of in vitro cells. It is believed that this will be particularly advantageous as a research tool to deliver these molecules into cell lines that are otherwise difficult to genetically manipulate.

In another aspect the disclosure provides compositions comprising: a translocation sequence comprising a polypeptide having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6; and a payload coupled to the translocation sequence, for use in a method of treatment of a mammalian subject.

In another aspect the disclosure provides compositions comprising: a translocation sequence comprising a polypeptide having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6; and a payload coupled to the translocation sequence, for use in a method of treatment of a human or animal subject, wherein the subject is not a fish.

As outlined above, previous work had incorrectly indicated that SpHtp1 translocation was fish specific, and that SpHtp1 derived translocation sequences were not able to direct translocation into mammalian cells. Here the authors demonstrate that SpHtp1 is able to translocate coupled payloads into mammalian cells such as human cells via the human β2-adrenoceptor. This unexpected finding advantageously expands the subject groups known to be treatable with SpHtp1-coupled payloads.

In humans, the β2-adrenoceptor is known to be highly expressed in bronchial smooth muscle and epithelial cells, as well as in cardiac myocytes and vascular smooth muscle cells. The compositions of the present disclosure are therefore particularly well suited to administration to by inhalational, injection (particularly intravenous), immersion, and oral routes. Accordingly, in some embodiments the subject is human. In some embodiments the compositions of the disclosure are administered by injection, inhalational, or oral routes. In some embodiments the method is a method of treatment of lung/respiratory disease.

The authors have previously demonstrated that the SpHtp1 translocation sequence can effectively translocate coupled payloads into fish cells both in vitro and in live fish. This translocation was effective enough to elicit an immune (antibody) response against the antigenic polypeptide. Here, the authors demonstrate that this translocation is effective in mammalian cells even for large, non-biological, payloads in the μm size range. Accordingly, in some embodiments the payload is: a marker or imaging agent; a polypeptide or nucleic acid; an antibody; an antibiotic agent; a lipid; a small organic molecule; a metal; a therapeutic agent, preferably a therapeutic agent useful in the treatment of lung/respiratory disease; a protective agent; or a cytotoxic agent. In some embodiments the payload is an antigen or immunogen. In other embodiments the payload is a particle.

The translocation sequence can be coupled to the payload by any means of bonding or otherwise associating the payload and the translocation sequence. In some embodiments the translocation sequence is bonded to the payload via a covalent, hydrogen, or electrostatic bond. In some embodiments the translocation sequence is associated with the payload via hydrophobic association or van der Waals interactions. In some embodiments the translocation sequence is covalently bonded to a peptide payload, for example, via a peptide bond. In such embodiments the composition may be a fusion protein.

As outlined above, the authors have demonstrated that SpHtp1 is able to enter human cells via the human β2-adrenoceptor. The authors have also demonstrated that the β2-adrenergic receptor inhibitor SCH-202676 can block translocation of the SpHtp1 translocation sequence into cells in a concentration-dependent manner. In doing so the authors have uncovered a new and advantageous therapeutic application for modulators of β-adrenergic receptors. The β2-adrenoceptor homologue in fish is highly homologous with the mammalian β2-adrenoceptor, thus it is very likely that SpHtp1 binds to the β2-adrenoceptor homologue in fish. Accordingly, in another aspect the disclosure provides a method of preventing infection of fish or other gilled animals by a Saprolegnia genus pathogen, the method comprising administering to a fish or other gilled animal a β-adrenergic receptor modulator, preferably a β2-adrenergic receptor modulator. In some embodiments the β-adrenergic receptor modulator is a 6-adrenergic receptor inhibitor, preferably a β2-adrenergic receptor inhibitor. In some embodiments the Saprolegnia genus pathogen is selected from: S. australis, S. ferax, S. diclina, S. delica, S. longicaulis, S. mixta, S. parasitica, S. sporangium, and/or S. variabilis.

Advantageously, this new therapeutic application extends to any fish or gilled animal the cells of which express β-adrenergic receptors. In some embodiments the gilled animal is a fish. In some embodiments the fish is a salmonid, catfish, carp, sea bass, flat fish, or Tilapia. In some embodiments the fish is a: Grass carp (Ctenopharyngodon idella), Silver carp (Hypophthalmichthys molitrix), catla (Cyprinus catla or Gibelion catla), Common Carp (Cyprinus carpio), Bighead carp (Hypophthalmichthys nobilis or Aristichthys nobilis), Crucian carp (Carassius carassius), Nile Tilapia (Oreochromis niloticus), Mozambique Tilapia (Oreochromis mossambicus), Pangas catfish (Pangasius pangasius), Roho (Labeo rohita), Atlantic salmon (Salmo salar), Arctic charr (Salvelinus alpinus), brown trout (Salmo trutta), rainbow trout (Oncorhynchus mykiss), or sea trout (Salmo trutta). In some embodiments the β-adrenergic receptor modulator is administered by the injection, immersion, or oral route.

In another aspect the disclosure provides a β-adrenergic receptor modulator for use in a method of preventing infection of fish or other gilled animals by a Saprolegnia genus pathogen.

In another aspect the disclosure provides the use of a β-adrenergic receptor modulator to inhibit or block translocation of a polypeptide across the plasma membrane of a eukaryotic cell, wherein the polypeptide comprises a translocation sequence having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6.

Once inside the cell, SpHtp1 is not confined to vesicles but has access to the cytosol. Surprisingly, the authors have shown here that SpHtp1 is also able to effect the release of other molecules from endocytosed vesicles. Accordingly, in a further aspect the present disclosure provides a method of enhancing the release of vesicle contents into the cytoplasmic compartment of a eukaryotic cell, the method comprising contacting the cell with a composition comprising a polypeptide having at least 20, 30, 40, 50, 60, 70, 75, 80, 85, 90, 95, 98, 99 or 100% sequence identity to the SpHtp1²⁴⁻¹⁹⁸ or SpHtp1a²⁴⁻²²¹ shown in FIG. 6, or a polypeptide having at least 20, 30, 40, 50, 60, 70, 75, 80, 85, 90, 95, 98, 99 or 100% sequence identity to the full-length SpHtp1¹⁻¹⁹⁸ or SpHtp1a¹⁻²²¹ shown in FIG. 6.

In another aspect the disclosure provides a method of delivering a composition to the cytoplasmic compartment of a eukaryotic cell, the method comprising: contacting the cell with the composition such that the composition enters the cell by endocytosis; and, contacting the cell with a composition comprising a polypeptide having at least 20, 30, 40, 50, 60, 70, 75, 80, 85, 90, 95, 98, 99 or 100% sequence identity to the SpHtp1²⁴⁻¹⁹⁸ or SpHtp1a²⁴⁻²²¹ shown in FIG. 6, or a polypeptide having at least 20, 30, 40, 50, 60, 70, 75, 80, 85, 90, 95, 98, 99 or 100% sequence identity to the full-length SpHtp1¹⁻¹⁹⁸ or SpHtp1a¹⁻²²¹ shown in FIG. 6.

In another aspect the disclosure provides a composition comprising a translocation sequence comprising a polypeptide having at least 20, 30, 40, 50, 60, 70, 75, 80, 85, 90, 95, 98, 99 or 100% sequence identity to the SpHtp1²⁴⁻¹⁹⁸ or SpHtp1a²⁴⁻²²¹ shown in FIG. 6, or a polypeptide having at least 20, 30, 40, 50, 60, 70, 75, 80, 85, 90, 95, 98, 99 or 100% sequence identity to the full-length SpHtp1¹⁻¹⁹⁸ or SpHtp1a¹⁻²²¹ shown in FIG. 6, for use in a method of treatment of the human or animal body, wherein the treatment comprises administering the composition to a subject in order to enhance the release of the contents of endocytic vesicles into the cytosol of a eukaryotic cell.

In another aspect the disclosure provides the use of a translocation sequence comprising a polypeptide having at least 20, 30, 40, 50, 60, 70, 75, 80, 85, 90, 95, 98, 99 or 100% sequence identity to the SpHtp1²⁴⁻¹⁹⁸ or SpHtp1a²⁴⁻²²¹ shown in FIG. 6, or a polypeptide having at least 20, 30, 40, 50, 60, 70, 75, 80, 85, 90, 95, 98, 99 or 100% sequence identity to the full-length SpHtp1¹⁻¹⁹⁸ or SpHtp1a¹⁻²²¹ shown in FIG. 6 to enhance the release of vesicle contents into the cytosolic compartment of a eukaryotic cell.

In some embodiments the vesicles are endocytic vesicles. In some embodiments the vesicle contents is a composition co-administered to the cell with the composition comprising a polypeptide having at least 20, 30, 40, 50, 60, 70, 75, 80, 85, 90, 95, 98, 99 or 100% sequence identity to the SpHtp1²⁴⁻¹⁹⁸ or SpHtp1a²⁴⁻²²¹ shown in FIG. 6, or a polypeptide having at least 20, 30, 40, 50, 60, 70, 75, 80, 85, 90, 95, 98, 99 or 100% sequence identity to the full-length SpHtp1¹⁻¹⁹⁸ or SpHtp1a¹⁻²²¹ shown in FIG. 6. This newly discovered effect of SpHtp1 is envisaged as being particularly beneficial in effecting the release of, e.g. co-administered drug molecules, from endocytosed vesicles. A common problem with drug treatments is that while these can be delivered into cells, they remain trapped inside endocytosed vesicles and are unable to enter the cytoplasmic compartment to fulfil their therapeutic function.

The disclosure includes the combination of the aspects and preferred features described except where such a combination is clearly impermissible or expressly avoided.

### SUMMARY OF THE FIGURES

Embodiments and experiments illustrating the principles of the disclosure will now be discussed with reference to the accompanying figures in which:

FIG. 1 shows translocation of μm scale particles into human cells by SpHtp1. Particles (Dynabeads® microspheres; 6 μm) were coupled to SpHtp1²⁴⁻⁶⁸ and incubated with human A549 cells for 1 h at 37° C. Translocation into the cell can be observed as a dent in the cell nucleus (indicated by arrowhead).

FIG. 2 shows GPCR-mediated translocation of SpHtp1 into fish and human cells. (A) The fusion construct SpHtp1²⁴⁻⁶⁸-mRFP translocates into human A549 cells; the results shown are following a 1 h incubation at 37° C. (B) RTG-2 cells incubated with SpHtp1²⁴⁻⁶⁸-mRFP were co-incubated with compounds known to inhibit different uptake pathways into cells. Brefeldin A (an inhibitor of caveolae-mediated endocytosis) and nystatin (an inhibitor of lipid raft-mediated endocytosis) had no effect on the translocation of SpHtp1²⁴⁻⁶⁸-mRFP. Conversely, dynasore (an inhibitor of clathrin-mediated endocytosis) clearly resulted in the accumulation of red fluorescence at the cell surface without entering the cells, indicating that SpHtp1 is taken up by clathrin-mediated endocytosis. (C) The translocation of SpHtp1²⁴⁻⁶⁸-mRFP is a time-dependent process. Scale bars: 10 μM.

FIG. 3 shows data demonstrating that the β-adrenoreceptor mediates uptake of SpHtp1 into cells. (A) The translocation of SpHtp1²⁴⁻⁶⁸-mRFP into human A549 cells is inhibited by pre-incubation of the cells with a β-adrenergic inhibitor (10 μM SCH-202676). Binding of SpHtp1²⁴⁻⁶⁸-mRFP to the cells is not influenced. (B) The inhibition of the translocation of SpHtp1²⁴⁻⁶⁸-mRFP is concentration dependent—inhibition of translocation increases with increasing concentration of the β-adrenergic inhibitor SCH-202676. (C) Co-immunoprecipitation experiments with an antibody against a β2-adrenergic receptor indicate an interaction between the receptor molecule and SpHtp1. Scale bars: 10 μM.

FIG. 4 demonstrates binding of SpHtp1²⁴⁻⁶⁸-mRFP to isolated mitochondria. SpHtp1²⁴⁻⁶⁸-mRFP and mRFP only (as control) were incubated with isolated mitochondria from RTG-2 cells for 1 h at RT. The membrane-associated ion channel VDAC was used as a mitochondria marker. After incubation mitochondria were collected by centrifugation and analysed by immune blot. While mRFP can only be found in the supernatant, SpHtp1 is also found in the centrifuged pellet associated to mitochondria

FIG. 5 shows data demonstrating SpHtp3 release from vesicles by SpHtp1. (A) RTG-2 cells in direct contact with S. parasitica appear shrunken with a condensed nucleus. In these cells no cytosolic RNA (SytoRNA) can be detected and infected cells contain a high number of vesicles (membrane stain FM4-64FX). In contrast, cells in close proximity but no direct contact do not show any morphological abnormalities. (B) Trout RTG-2 cells (c) were challenged with S. parasitica (h) after 1 h incubation with SpHtp3-mRFP. A hyphal tip (arrowhead, DIC) is attacking an RTG-2 cell. Magnification of the infected cell (square) at different time points (bottom) show vesicles disappearing within a minute (arrowheads). In contrast, cells in close proximity but with no direct contact to S. parasitica contain less disappearing vesicles (C) Quantification of SpHtp3-mRFP containing vesicles of RTG-2 cells from (B) over time. (D) Vesicle release of SpHtp3-mRFP into the cytosol of RTG-2 cells after pre-incubation with SpHtp1²¹⁻¹⁹⁸ His6 at pH 7.5. SpHtp3 accumulates in vesicles of RTG-2 cells after self-translocation. However, after co-incubation of SpHtp1 with SpHtp3, the number of vesicles in the periphery of the cells is reduced and the cytosolic fluorescence of RFP increased. Fluorescence intensity of SpHtp3-mRFP across the cell as indicated by dashed lines. Scale bars: 20 μm. Pictures were taken with a Zeiss Imager M2. (E) In vitro complex formation of recombinant SpHtp1-His6 and SpHtp3-His6 after cross link verified by LC-MS/MS. An additional band which only appears in the sample with both proteins is highlighted (Complex).

FIG. 6 shows SpHtp1 and SpHtp1a sequences (“RxLR” motif shown in bold).

### DETAILED DESCRIPTION OF THE DISCLOSURE

Aspects and embodiments of the present disclosure will now be discussed with reference to the accompanying figures. Further aspects and embodiments will be apparent to those skilled in the art. All documents mentioned in this text are incorporated herein by reference.

The disclosure provides compositions comprising, consisting, or consisting essentially of a translocation sequence and a payload coupled to the translocation sequence. In some embodiments the payload is a particle. The disclosure also provides compositions comprising, consisting, or consisting essentially of an inactivated virus, bacteria, fungus, or other disease causing microbe wherein the inactivated virus, bacteria, fungus, or other disease causing microbe expresses on its surface a polypeptide comprising a translocation sequence. The translocation sequence enhances the translocation of the payload across the plasma membrane of a eukaryotic cell, for example a fish cell or mammalian cell.

Translocation Sequence

The oomycetes are a phylum of eukaryotes containing many pathogenic species that utilise effector proteins to modulate host cell molecular processes in order to establish an infection and/or suppress immune response in these host cells. A tetrameric amino acid sequence motif, RxLR (Arg, any amino acid, Leu and Arg), is known to be common to several characterised oomycete effector proteins. This sequence is believed to be essential for directing the protein to the appropriate site for secretion out of the pathogen, and positioning the effector at the appropriate location for translocation into the target host. This group of effector proteins are commonly referred to as RxLR effector proteins.

The authors have previously shown that translocation of RxLR effector proteins is not directly mediated by the RxLR-motif, but rather by the effector domain of oomycete RxLR effector proteins in a pathogen-independent manner. Thus, an RxLR sequence, or sequence comprising an RxLR motif, refers to a sequence present in a group of effector proteins which can translocate into host cells. The mechanisms by which RxLR effector proteins translocate into cells are thought to be similar to those of the Plasmodium PEXEL system.

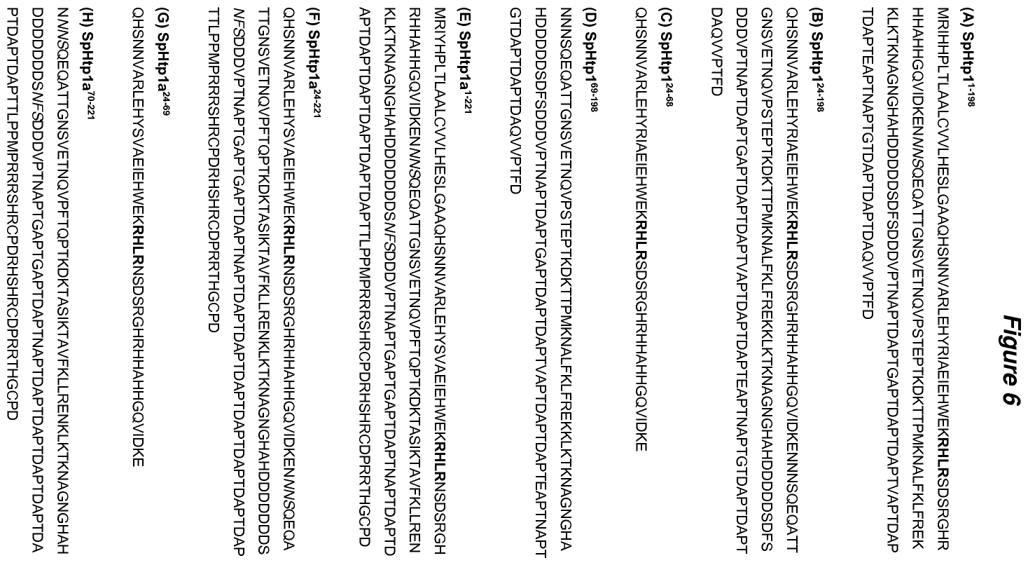

The authors have previously shown that payloads coupled to the S. parasitica derived SpHtp1 effector protein can be effectively delivered across the lipid membrane of eukaryotic cells, both in vitro and in live fish (WO 2011/148135; WO 2014/191759). Furthermore, as shown by the authors in WO 2011/148135, WO 2014/191759, and the present examples, payloads coupled to peptide fragments of SpHtp1, for example fragments having the sequence of SpHtp1²⁴⁻⁶⁸ or SpHtp1a²⁴⁻⁶⁹ as shown in FIG. 6, remain capable of translocating across the membrane of eukaryotic cells.

Thus, the disclosure provides compositions comprising, consisting, or consisting essentially of a translocation sequence and a payload coupled to the translocation sequence. Translocation sequences are capable of enhancing the translocation of a coupled payload across the membrane of a eukaryotic cell. In compositions of the disclosure comprising a translocation sequence, the translocation sequence enhances translocation of the composition across the membrane of a eukaryotic cell. As used herein, translocation refers to the movement of a translocation sequence and coupled payload across the membrane of a eukaryotic cell.

In some embodiments the translocation sequence is an SpHtp1 translocation sequence. As used herein, an “SpHtp1 translocation sequence” or “SpHtp1 derived translocation sequence” is a sequence comprising an SpHtp1 effector protein or fragment thereof, and which is capable of translocating across the membrane of eukaryotic cells.

In some preferred embodiments the translocation sequence may comprise, consist, or consist essentially of a polypeptide having the sequence of SpHtp1²⁴⁻⁶⁸ or SpHtp1a²⁴⁻⁶⁹ as shown in FIG. 6. In some embodiments the translocation sequence may comprise, consist, or consist essentially of a polypeptide having the sequence of SpHtp1²⁴⁻¹⁹⁸ or SpHtp1a²⁴⁻²²¹ shown in FIG. 6.

SpHtp1 is a putative effector from the fish pathogenic oomycete, Saprolegnia parasitica. The amino acid sequences of ‘full-length’ SpHtp1 (1-198) and a number of fragments are shown in FIG. 6.

“Full-length” SpHtp1 contains 198 amino acids (see FIG. 6). However the fragment consisting of amino acids 24 to 68 (SpHtp1²⁴⁻⁶⁸) fused to mRFP has previously been shown by the authors to retain the ability to translocate into both in vitro cells and into the cells of living fish in vivo. Previous work had incorrectly indicated that this effect was specific to fish cells (Wawra et al. 2012, PNAS Vol 109 (6) pp 2096-2101; Wawra et al. 2012, MPMI Vol 26 (5) pp 528-36; WO 2014/191759). Surprisingly, the present application shows that, contrary to this previous work, SpHtp1 is also able to translocate into human cells (such as HEK cells or the human epithelial lung cell line A549)—see Example 2.

The SpHtp1 homolog, SpHtp1a, also has this translocation ability. Thus, in some embodiments the translocation sequence consists of, or consists essentially of, SpHtp1²⁴⁻⁶⁸ or SpHtp1a²⁴⁻⁶⁹ as shown in FIG. 6.

The authors have found that SpHtp1 and fragments thereof do not have any immediate cytotoxic effects on cells, and do not alter cell morphology or viability, even with extended incubation periods of up to 24 hours.

Longer polypeptides comprising the SpHtp1²⁴⁻⁶⁸ or SpHtp1a²⁴⁻⁶⁹ fragments also have the ability to translocate into eukaryotic cells. Thus, in some embodiments the translocation sequence comprises SpHtp1²⁴⁻⁶⁸ or SpHtp1a²⁴⁻⁶⁹ as shown in FIG. 6. The translocation sequence may be of any length. However, in some embodiments it is no more than 200 amino acids, such as no more than 150, no more than 125, no more than 100, or no more than 75 amino acids. In some embodiments the translocation sequence is no more than 60 amino acids. In some embodiments the translocation sequence is no more than 50 amino acids.

Where the SpHtp1²⁴⁻⁶⁸ or SpHtp1a²⁴⁻⁶⁹ as shown in FIG. 6 forms part of a longer translocation sequence, the SpHtp1²⁴⁻⁶⁸ or SpHtp1a²⁴⁻⁶⁹ fragment can be at any point of the translocation sequence: for example, at the very N-terminal or at the very C-terminal.

Sequence variants of SpHtp1²⁴⁻⁶⁸ are also able to translocate into eukaryotic cells. Thus, in some embodiments the translocation sequence consists of, or consists essentially of, a sequence variant of SpHtp1²⁴⁻⁶⁸ shown in FIG. 6. For example, the SpHtp1 homolog SpHtp1a shown in FIG. 6 is able to translocate into both fish and human cells. Thus, in some embodiments the translocation sequence consists of, or consists essentially of, a sequence variant of SpHtp1²⁴⁻⁶⁸ shown in FIG. 6.

Longer polypeptides comprising sequence variants of the SpHtp1²⁴⁻⁶⁸ or SpHtp1a²⁴⁻⁶⁹ shown in FIG. 6 have the ability to translocate into eukaryotic cells. Thus, in some embodiments the translocation sequence comprises a sequence variant of SpHtp1²⁴⁻⁶⁸ or SpHtp1a²⁴⁻⁶⁹ shown in FIG. 6. The translocation sequence may be of any length. However, in some embodiments it is no more than 200 amino acids, such as no more than 150, no more than 125, no more than 100 or no more than 75 amino acids. In some embodiments the translocation sequence is no more than 60 amino acids. In some embodiments the translocation sequence is no more than 50 amino acids.

Where the sequence variant of SpHtp1²⁴⁻⁶⁸ or SpHtp1a²⁴⁻⁶⁹ shown in FIG. 6 forms part of a longer translocation sequence, the variant SpHtp1²⁴⁻⁶⁸ or SpHtp1a²⁴⁻⁶⁹ fragment can be at any point of the translocation sequence: for example, at the very N-terminal or at the very C-terminal.

As used herein, a “sequence variant of SpHtp1²⁴⁻⁶⁸ or SpHtp1a²⁴⁻⁶⁹ shown in FIG. 6” is an amino acid sequence having at least 60, 70, 75, 80, 85, 90, 95, 96, 97, 98, 99 or 100% identity to the amino acid sequence SpHtp1²⁴⁻⁶⁸ or SpHtp1a²⁴⁻⁶⁹ shown in FIG. 6. A “sequence variant of SpHtp1²⁴⁻¹⁹⁸ or SpHtp1a²⁴⁻²²¹ shown in FIG. 6” is an amino acid sequence having at least 20, 30, 40, 50, 60, 70, 75, 80, 85, 90, 95, 98, 99 or 100% identity to the sequence SpHtp1²⁴⁻¹⁹⁸ or SpHtp1a²⁴⁻²²¹ shown in FIG. 6. A “sequence variant of full-length SpHtp1¹⁻¹⁹⁸ or SpHtp1a¹⁻²²¹ shown in FIG. 6” is an amino acid sequence having at least 20, 30, 40, 50, 60, 70, 75, 80, 85, 90, 95, 98, 99 or 100% identity to the sequence of the full-length SpHtp1¹⁻¹⁹⁸ or SpHtp1a¹⁻²²¹ shown in FIG. 6.

In one embodiment the translocation sequence will have equal to, or no more than, 1, 2, 3, 4, 5, 6, 7, 8, 9, 10, 12, 15 or 20 substitutions, deletions, or additions in the amino acid sequence SpHtp1²⁴⁻⁶⁸ or SpHtp1a²⁴⁻⁶⁹ shown in FIG. 6.

Identity may be as defined using sequence comparisons are made using FASTA and FASTP (see Pearson & Lipman, 1988. Methods in Enzymology 183: 63-98). Parameters are preferably set, using the default matrix, as follows: Gapopen (penalty for the first residue in a gap): −12 for proteins/−16 for DNA; Gapext (penalty for additional residues in a gap): −2 for proteins/−4 for DNA; KTUP word length: 2 for proteins/6 for DNA.

For the avoidance of doubt, the level of sequence identity is measured over the full-length of the amino acid sequence, for example the full-length of SpHtp1²⁴⁻⁶⁸ or SpHtp1a²⁴⁻⁶⁹ shown in FIG. 6 (45 or 46 amino acids).

The variant translocation sequences may originate from other native proteins, or may be prepared by those skilled in the art, for example by site directed or random mutagenesis of the SpHtp1²⁴⁻⁶⁸ or SpHtp1a²⁴⁻⁶⁹ shown in FIG. 6. Alternatively the variant SpHtp1 translocation sequences may be produced by direct synthesis.

Changes may be desirable for a number of reasons, including introducing or removing the following features: sites which are required for post translation modification; cleavage sites in the encoded polypeptide; motifs in the encoded polypeptide (e.g. epitopes). Leader or other targeting sequences (e.g. hydrophobic anchoring regions) may be added or removed from the expressed protein to determine its location following expression.

Other desirable mutations may be made by random or site directed mutagenesis of the nucleic acid encoding the polypeptide in order to alter the activity (e.g. specificity) or stability of the encoded polypeptide.

Changes may be by way of conservative variation, i.e. substitution of one hydrophobic residue such as isoleucine, valine, leucine or methionine for another, or the substitution of one polar residue for another, such as arginine for lysine, glutamic for aspartic acid, or glutamine for asparagine. As is well known to those skilled in the art, altering the primary structure of a polypeptide by a conservative substitution may not significantly alter the activity of that peptide because the side chain of the amino acid which is inserted into the sequence may be able to form similar bonds and contacts as the side chain of the amino acid which has been substituted out. This is so even when the substitution is in a region which is critical in determining the peptides conformation.

Also included are variants having non conservative substitutions. As is well known to those skilled in the art, substitutions to regions of a peptide which are not critical in determining its conformation may not greatly affect its ability to raise antibodies because they do not greatly alter the peptide's three dimensional structure.

In regions which are critical in determining the peptides conformation or activity such changes may confer advantageous properties on the polypeptide. Indeed, changes such as those described above may confer slightly advantageous properties on the peptide e.g. altered specificity, stability or immunogenicity.

Payload

The payload may be any type of molecule, for example a polypeptide, nucleic acid, lipid, or small organic molecule. The authors have demonstrated, in WO 2011/148135 and WO 2014/191759, that SpHtp1 translocation sequence-coupled payloads such as antigenic peptides can be effectively translocated into eukaryotic cells both in vitro and in vivo in live fish. In WO 2014/191759 this translocation was shown to be effective enough to elicit an immune (antibody) response against the antigenic polypeptide.

The payload may be a molecule which the SpHtp1 translocation sequence is not associated with in its native setting. For example, the payload may be an “exogenous protein” i.e. a protein that is not natively expressed in the eukaryotic cell into which it is being translocated, or the payload may be a “particle”. Exogenous proteins include, for example, fusions of native proteins, or fragments of native proteins, with non-native polypeptides or other molecules. The payload may be molecules which are not fragments of a translocation sequence, for example molecules which are not fragments of SpHtp1.

In some embodiments the payload is a marker or imaging agent, for example a fluorescent protein such as GFP or RFP, or a fluorescent molecule or fluorophore such as fluorescein (FITC).

In some embodiments the payload is a peptide or polypeptide. In embodiments where the payload is a peptide or polypeptide, the translocation sequence may be of any length. However, in some embodiments it is no more than 500 amino acids, such as no more than 400, no more than 300, no more than 200 or no more than 100 amino acids. In some embodiments the translocation sequence is no more than 75 amino acids. In some embodiments the translocation sequence is no more than 50 amino acids, such as no more than 30 amino acids. The terms “peptide”, “polypeptide” and “protein” as used herein are used interchangeably to mean a polymer of two or more amino acids coupled through peptide bonds.

The payload may be an “exogenous protein” i.e. a protein that is not natively expressed in the eukaryotic cell into which it is being translocated. Exogenous proteins include, for example, fusions of native proteins, or fragments of native proteins, with non-native polypeptides or other molecules.

In some embodiments the payload is a nucleic acid, an antibody, an antibiotic agent, a lipid, a small organic molecule, or a metal. In some embodiments the payload is an RNA molecule such as an siRNA or miRNA.

In some embodiments the payload is a therapeutic agent. In some embodiments the payload is a therapeutic agent useful in the treatment of lung/respiratory disease, for example cystic fibrosis, chronic obstructive pulmonary disease (COPD), asthma, lung cancer, or bacterial, viral, or fungal lung infection. Therapeutic agents useful in the treatment of lung/respiratory disease include nucleic acids, proteins, and small molecule agents.

Exemplary therapeutic agents useful in the treatment of lung/respiratory disease include: β2-adrenergic receptor agonists, M3 muscarinic receptor antagonists, dual β2-adrenoceptor agonists/M3 muscarinic receptor antagonists, glucocorticoid receptor agonists, leukotriene antagonists, 5-lipoxygenase inhibitors, cromones, immunosuppressants, immune response modifiers (e.g. agonists of one or more Toll-Like Receptors) or vaccines, xanthine derivatives, selective phoshodiesterase (PDE) isoenzyme inhibitors (e.g., inhibitors of PDE4 and/or PDE5), inhibitors of certain kinase enzymes (e.g. p38 mitogen-activated protein (MAP) kinase, IkappaB kinase 2 (IKK2), tyrosine-protein kinase (Syk), and phosphoinositide-3 kinase gamma (PI3Kgamma)), histamine type 1 receptor antagonists, a adrenoceptor agonist vasoconstrictor sympathomimetics, inhibitors of a matrix metalloprotease, modulators of chemokine receptor function, cytokines, modulators of cytokine function, agents which act on a cytokine signalling pathway, immunoglobulins, immunoglobulin preparations, antagonists that modulate immunoglobulin function, antibodies that modulate immunoglobulin function, lung surfactant proteins (especially SP-A and SP-D), inhibitors of Der p 3, inhibitors of Der p 6, and inhibitors of Der p 9.

In some embodiments the payload is a protective agent (i.e. an agent that protects a target cell from the effects of a negative stimulus to which the cell is subsequently exposed). Protective agents include, for example, chemoprotective and radioprotective agents (which protect healthy tissue from the toxic effects of anticancer drugs and radiation therapy), neuroprotective agents, antimutagenic agents, and antioxidant agents.

In some embodiments the payload is a cytotoxic molecule (i.e. a molecule, which when bound to or taken up by a target cell stimulates the death and lysis of the cell). Cytotoxic molecules include members of the following groups or families: nitrogen—mustard types (e.g. melphalan), anthracyclines (e.g. adriamycin, doxorubicin, and daunomycin), nucleoside analogues (e.g. cytosine arabinoside) or antimetabolites (e.g. methotrexate). Also encompassed by the term “cytotoxic molecules” as used herein are enzymes intended to catalyse the conversion of a non-toxic prodrug into a cytotoxic drug (for example a HSV-Thymidine Kinase/Ganciclovir system). The prodrug may be systemically administered.

The authors have previously shown that payloads of these sorts can be effectively delivered across the lipid membrane of eukaryotic cells when coupled to a translocation sequence such as an SpHtp1 translocation sequence, both in vitro and in vivo in live fish (WO 2011/148135; WO 2014/191759).

In some preferred embodiments the payload is a particle or bead.

Particles/Beads

The authors have now surprisingly shown that SpHtp1 translocation sequences are capable of enhancing the translocation of coupled large particle payloads across the lipid membrane of eukaryotic cells. As shown in Example 1, particles in the μm size range (for example microbeads and microspheres such as Dynabeads® and Fluoresbrite®) coated with SpHtp1 derived translocation sequences can be translocated into eukaryotic cells. These particles are a number of orders of magnitude larger in size (and even larger again in volume) than the protein and small molecule payloads previously shown to be translocated into cells by SpHtp1 translocation sequences. Nor are these particles formed of biological material in the same way as previously investigated payloads. This surprising discovery opens the possibility of delivering any molecule of interest into eukaryotic cells, as outlined in the following.

Particles as described herein are those having a nanomeric or micromeric scale. The term “particle” as used herein thus refers to a particle between 0.01 and 100 μm in size, that is, between 10 and 100,000 nm in size. These are not limited to particles having any specific shape or composition. In some embodiments the particles described herein have a generally polyhedral or spherical geometry.

In some embodiments the particles are microparticles. That is, in some embodiments the particles are 0.1-100 μm in size. Microparticles may also be referred to as micro-beads, or micro-spheres. In some embodiments the particles are nanoparticles. That is, in some embodiments the particles are 1-100 nm in size. Nanoparticles may also be referred to as nano-beads, or nano-spheres.

In some embodiments the particles described herein are between 10 and 10,000 nm in size. In some embodiments the particles described herein are between 20 and 6000 nm in size, or are between 100 and 1000 nm in size.

In some embodiments the particles are at least 10, 20, 30, 40, 50, 60, 70, 80, 90 or 100 nm in size. In other embodiments the particles are at least 200, 300, 400, 500, 600, 700, 800, 900, or 1000 nm in size. In other embodiments the particles are at least 1000, 2000, 3000, 4000, 5000, 6000, 7000, 8000, 9000, or 10,000 nm in size.

Preferably, the particles are at least 10 nm in size. The authors have unexpectedly found that the translocation sequence SpHtp1²⁴⁻⁶⁸ is able to translocate particles in the μm size range into eukaryotic cells—see Example 1, where translocation of 6 μm particles is demonstrated.

In some embodiments the particles are less than 100, 90, 80, 70, 60, 50, 40, 30, 20, or 10 μm in size. In other embodiments the particles are less than 10,000, 9000, 8000, 7000, 6000, 5000, 4000, 3000, 2000, or 1000 nm in size. In other embodiments the particles are less than 900, 800, 700, 600, 500, 400, 300, 200, or 100 nm in size.

The particles described herein are not limited to those having any specific shape or composition.

In some embodiments the particles are rod-shaped, polyhedral, or spherical. In some embodiments the particles are non-enclosed containers or clamps, for example DNA containers (see Sprengel et al, 2017), molecular tweezers (see Trusch et al, 2016), molecular clips, or other molecules having an open cavity capable of binding or encapsulating one or more cargo molecule.

In some embodiments the particles are imaging agents having high contrast in detection methods, for example a metallic particle, gold particle, semiconductor particle (for example a quantum dot), or fluorescent particle. In some embodiments the particles are magnetic particles, for example Dynabeads®. In some embodiments the particles are polymeric or liposomal nanoparticles, or have an outer membrane comprising lipid, plastic, or other synthetic or natural polymer. In some embodiments the particles comprise both an outer and inner membrane (bilayer), while in other embodiments the particles comprise only an outer membrane. The outer membrane may in some cases be referred to as the outer surface. Examples of polymeric nanoparticles include micelles, capsules, platelets, fibres, spheroids, colloids, dendrimers, core-shells, and nanoparticle incorporated polymer matrixes.

In some embodiments the nanoparticles are viruses or viral particles (also called virions). Preferably the viral particles are inactivated. In some embodiments the nanoparticles are bacteria, fungii, or other disease causing microbes. Preferably the bacteria, fungii, or other disease causing microbes are inactivated. In such embodiments the virus, bacteria, fungus, or other disease causing microbe may express an SpHtp1 translocation sequence on its surface. In some preferred embodiments the virus, bacteria, fungus, or other disease causing microbe expresses on its surface a polypeptide comprising a translocation sequence having at least 60, 70, 75, 80, 85, 90, 95, 98, 99 or 100% sequence identity to the SpHtp1²⁴⁻⁸⁸ or SpHtp1a²⁴⁻⁶⁹ shown in FIG. 6.

In some embodiments the nanoparticles have an outer membrane comprising lipid, plastic, or other synthetic or natural polymers enclosing one or more interior cavities. In some embodiments the nanoparticles encapsulate or contain one or more cargo molecules.

In some embodiments, the particles described herein encapsulate or contain one or more cargo molecules. The term “cargo molecule” as used herein refers to a molecule encapsulated or contained within a particle, and for which the particle acts as a carrier to translocate the cargo molecule across the membrane of a eukaryotic cell.

The cargo molecule may be any type of molecule which can be encapsulated or contained within a particle, for example a polypeptide, nucleic acid, lipid, or small organic molecule. In some embodiments, the cargo molecules are less than 100, 90, 80, 70, 60, 50, 40, 30, 20, or 10 nm in size. In other embodiments the cargo molecules are less than 10, 9, 8, 7, 6, 5, 4, 3, 2, or 1 nm in size. In some embodiments, the cargo molecules have a molecular weight less than 1000, 900, 800, 700, 600, 500, 400, 300, 200, or 100 g/mol.

In some embodiments the particles encapsulate or contain at least 50, 100, 200, 300, 400, 500, 600, 700, 800, 900, or 1000 cargo molecules. In some embodiments the particles encapsulate or contain at least 1000, 2000, 3000, 4000, 5000, 6000, 7000, 8000, 9000, or 10,000 cargo molecules. In some embodiments the particles encapsulate or contain at least 100,000, 500,000, or 1,000,000 cargo molecules. In some embodiments the particles encapsulate or contain at least 1×10⁷, 1×10⁸, 1×10⁹, 1×10¹⁰, 1×10¹¹, 1×10¹², 1×10¹³, 1×10¹⁴, or 1×10¹⁵ cargo molecules.

In some embodiments the cargo molecule is a marker or imaging agent, for example a fluorescent protein such as GFP or RFP, or a fluorescent molecule or fluorophore such as fluorescein (FITC). In some embodiments the cargo molecule is a peptide, polypeptide, or nucleic acid. In some embodiments the cargo molecule is an antibody, an antibiotic agent, a lipid, a small organic molecule, or a metal. In some embodiments the cargo molecule is an RNA molecule such as an siRNA or miRNA.

In some embodiments the cargo molecule is a therapeutic agent. In some preferred embodiments the cargo molecule is a therapeutic agent useful in the treatment of lung/respiratory disease, for example cystic fibrosis, chronic obstructive pulmonary disease (COPD), asthma, lung cancer, or bacterial, viral, or fungal lung infection. Therapeutic agents useful in the treatment of lung/respiratory disease include nucleic acid, protein, and small molecule agents.

Exemplary therapeutic agents useful in the treatment of lung/respiratory disease include: β-adrenergic receptor agonists, M3 muscarinic receptor antagonists, dual β-adrenoceptor agonists/M3 muscarinic receptor antagonists, glucocorticoid receptor agonists, leukotriene antagonists, 5-lipoxygenase inhibitors, cromones, immunosuppressants, immune response modifiers (e.g. agonists of one or more Toll-Like Receptors) or vaccines, xanthine derivatives, selective phoshodiesterase (PDE) isoenzyme inhibitors (e.g., inhibitors of PDE4 and/or PDE5), inhibitors of certain kinase enzymes (e.g. p38 mitogen-activated protein (MAP) kinase, IkappaB kinase 2 (IKK2), tyrosine-protein kinase (Syk), and phosphoinositide-3 kinase gamma (PI3Kgamma)), histamine type 1 receptor antagonists, a adrenoceptor agonist vasoconstrictor sympathomimetics, inhibitors of a matrix metalloprotease, modulators of chemokine receptor function, cytokines, modulators of cytokine function, agents which act on a cytokine signalling pathway, immunoglobulins, immunoglobulin preparations, antagonists that modulate immunoglobulin function, antibodies that modulate immunoglobulin function, lung surfactant proteins (especially SP-A and SP-D), inhibitors of Der p 3, inhibitors of Der p 6, and inhibitors of Der p 9.

In some embodiments the cargo molecule is a protective agent (i.e. an agent that protects a target cell from the effects of a negative stimulus to which the cell is subsequently exposed). Protective agents include, for example, chemoprotective and radioprotective agents (which protect healthy tissue from the toxic effects of anticancer drugs and radiation therapy), neuroprotective agents, antimutagenic agents, and antioxidant agents.

In some embodiments the cargo molecule is a cytotoxic molecule (i.e. a molecule, which when bound to or taken up by a target cell stimulates the death and lysis of the cell). Cytotoxic molecules include members of the following groups or families: nitrogen—mustard types (e.g. melphalan), anthracyclines (e.g. adriamycin, doxorubicin, and daunomycin), nucleoside analogues (e.g. cytosine arabinoside) or antimetabolites (e.g. methotrexate). Also encompassed by the term “cytotoxic molecules” as used herein are enzymes intended to catalyse the conversion of a non-toxic prodrug into a cytotoxic drug (for example a HSV-Thymidine Kinase/Ganciclovir system). The prodrug may be systemically administered.

In some preferred embodiments the cargo molecule is an antigen, immunogen, or vaccine.

In some preferred embodiments of the compositions of the disclosure, the payload comprises an antigen or immunogen.

Antigens and Immunogens

In some embodiments of the compositions of the disclosure the payload or cargo molecule comprises or consists of an antigen or immunogen. In principle, the payload or cargo molecule can comprise an antigen from any pathogen.

In some embodiments the payload or cargo molecule consists of a single antigen. In other embodiments, the payload or cargo molecule comprises one, two, three, four, five, six, seven, eight, or more than eight antigens. In embodiments where the payload or cargo molecule comprises more than one antigen, each antigen can be from the same or different organisms. In one embodiment, each antigen in the payload or cargo molecule is from a different organism (i.e. antigens 1, 2, 3, 4 are correspondingly form organisms A, B, C, D). When the payload comprises two or more antigens, the antigens may be separated by a short linker sequence or residue in order to facilitate expression or folding e.g. one, two, three, four, five or more gly residues.

As used herein, the term “antigen” is used to describe a substance that induces an immune response in an organism into which it is introduced—for example, the production of one or more antibodies (as detectable by techniques such as ELISA).

An antigen may be an inactivated virus, bacteria, fungus, or other disease causing microbe. An antigen may be a peptide, lipid or nucleic acid. The antigen may be a specific type or group of molecule. For example, the antigen may be a viral coat protein, a viral envelope protein, a viral lipid, a viral glycan, a bacterial envelope protein, a bacterial coat protein, a bacterial lipid, and/or a bacterial glycan.

An antigen may contain only a single epitope. Alternatively, in some embodiments an antigen contains two, three, four, five or more than 5 epitopes.

The antigen may be from a human pathogen. For example, the antigen may be a peptide or polypeptide from a human pathogen, or the antigen may be an inactivated human pathogen. Example human pathogens from which antigens may be from are (i) bacterial pathogens, for example Streptococcus pneumoniae, Haemophilus influenza type b (Hib), Corynebacterium diphtheriae, Bordetella pertussis, and Clostridium tetani; (ii) viral pathogens, for example human papillomavirus (HPV), poliovirus, influenza virus, herpes virus including varicella zoster virus, hepatitis A virus, hepatitis B virus, and rotavirus; (iii) Fungal pathogens, for example Candida albicans; and/or (iv) Parasitic pathogens, for example Plasmodium falciparum.

The antigen may be from a fish pathogen. For example, the antigen may be a peptide or polypeptide from a fish pathogen. Example fish pathogens from which antigens may be from are (i) bacterial pathogens, for example Vibrio anguillarum & Vibrio salmonicida (vibriosis), Aeromonas salmonicida & Aeromonas hydrophila (furunculosis), Yersinia ruckeri (Enteric Redmouth Disease), Renibacterium salmonis, Lactococcus sp., Streptococcus sp., and Piscirickettsia salmonis; (ii) viral pathogens, for example Infectious pancreatic necrosis virus (IPNV), Infectious Salmon Anemia virus (ISAV), Salmon Pancreas Disease virus (SPD virus), Sleeping Disease of Trout virus (SDV), Viral Nervous Necrosis virus (VNNV) and Infectious Heamatopoietic Necrosis virus (IHNV), Viral haemorrhagic septicaemia virus (VHSV); (iii) Fungal pathogens, for example Saprolegnia sp. (such as S. parasitica, S. diclina and S. australis) or Aphanomyces sp. (A. invadans and A. astaci) and Branchiomyces sp. (Gill rot); and/or (iv) Parasitic pathogens, for example Ichthyophthirius multifiliis, Cryptocaryon irritans and Trichodina sp. (Trichodiniasis), Amoebic gill disease (eg. Neoparamoeba perurans), sea lice (copepods within the order Siphonostomatoida, family Caligidae (for example Lepeophtheirus salmonis, Caligus rogercresseyi, Caligus clemensi, Caligus elongatus), proliferative kidney disease or PKD (Tetracapsuloides bryosalmonae) and other species.

The antigen may be a peptide or polypeptide from a virus, for example a peptide or polypeptide from the outer proteins or coat proteins of a virus such as IPNV (example polypeptides, or encoding nucleotides=Genbank accession numbers ACY35988.1, ACY35989.1, ACY35990.1; UniProt accession numbers D0VF01-1, D0VF02-1, D0VF03-1), ISAV (example polypeptides, or encoding nucleotides=Genbank accession numbers EU625675, FJ594325; UniProt accession numbers C6ETL2-1, C6G756-1), SPD (example polypeptides, or encoding nucleotides=Genbank accession number AJ012631.1; UniProt accession number Q9WJ34), SDV (example polypeptides, or encoding nucleotides=Genbank accession number AJ238578.1; UniProt accession number Q8QL52), IHNV (example polypeptides, or encoding nucleotides=Genbank accession number X89213.1; UniProt accession numbers Q08449-1, Q08455-1, Q08454-1, Q08453-1, Q82706-1, Q08455-1, Q08454-1, Q82707-1) VHSV (example polypeptides, or encoding nucleotides=Genbank accession numbers EU481506.1, ACA34520.1, ACA34521.1, ACA34522.1, ACA34523.1, ACA34524.1, ACA34525.1).

Coupling of Translocation Sequence and Payload

The compositions described herein comprise an SpHtp1 translocation sequence coupled to a payload. As used herein, “coupled to” can mean that the payload is bonded to, conjugated to, or linked to the translocation sequence via any one of a number of different bonds. As used herein, “coupled to” can also mean that the payload is associated with, or coated with the translocation sequence. For example, the payload and translocation sequence may be bonded together via a covalent, hydrogen, or electrostatic bond. Alternatively, the payload and translocation sequence may be associated together via hydrophobic association or van der Waals interactions. As described herein, when a translocation sequence is “coupled to” a payload, for example a particle, the translocation or delivery of the payload across the membrane of a eukaryotic cell is enhanced.

The coupling of the translocation sequence and the payload may be direct i.e. without intervening elements. That is, the translocation sequence may be bonded, or conjugated, directly to the payload.

The coupling of the translocation sequence and the payload may be indirect i.e. with intervening elements such as a linker or spacer molecule, or amino acid. That is, the translocation sequence may be bonded, or conjugated, to the payload through a linker or spacer molecule.

In some embodiments, the translocation sequence is bonded to a particle payload via an electrostatic bond. In some embodiments the translocation sequence carries an overall positive charge (that is, the sum of all positive and negative charges on the translocation sequence as a whole is positive) and the particle payload carries an overall negative charge (that is, the sum of all positive and negative charges on the particle as a whole is negative). In such embodiments the translocation sequence may be bonded to the beads by incubating the two together.

In embodiments where the payload is a particle, the composition of the disclosure is not a fusion protein.

In some embodiments, the translocation sequence is covalently bonded to a peptide payload, for example, via a peptide bond. In embodiments where the payload is a peptide or polypeptide the translocation sequence and the payload may be coupled via a peptide bond so that they form a contiguous polypeptide chain; that is, the composition of the disclosure may be a fusion protein comprising the translocation sequence fused to the peptide payload. The fusion protein of the disclosure may be of any length. However, in some embodiments it is no more than 500 amino acids, such as no more than 400, no more than 300, no more than 200 or no more than 100 amino acids. In some embodiments the translocation sequence is no more than 80 amino acids. In some embodiments the translocation sequence is no more than 60 amino acids.

In some embodiments the translocation sequence and peptide or nucleic acid payload may be separated by a short linker sequence or residue in order to facilitate expression or folding e.g. one, two, three, four, five or more gly residues. However, the presence of a linker is not required. Similarly, in embodiments where the payload contains two or more antigens, the antigens may be separated by a short linker sequence or residue in order to facilitate expression or folding e.g. one, two, three, four, five or more gly residues.

Preferred embodiments of the compositions of the disclosure include those in which the payload is a particle. Such embodiments comprise a particle and an SpHtp1 translocation sequence coupled to the particle.

In such embodiments the compositions comprise one or more translocation sequences coupled to the particle, wherein the number of translocation sequences coupled to the particle is sufficient to enhance translocation of the particle across the membrane of a eukaryotic cell. Thus in some embodiments the compositions comprise a plurality of SpHtp1 translocation sequences coupled to the particle, wherein the number of translocation sequences is sufficient to enhance translocation of the composition across the membrane of a eukaryotic cell. In some embodiments at least 1000, 2000, 3000, 4000, or 5000 translocation sequences are coupled to the particle. In some embodiments at least 10,000, 20,000, 30,000, 40,000, or 50,000 translocation sequences are coupled to the particle. In some embodiments at least 100,000, 200,000, 300,000, 400,000, or 500,000 translocation sequences are coupled to the particle.

In such embodiments the particles are coated with translocation sequences to a degree sufficient to enhance translocation of the particle across the membrane. Thus in some embodiments the particles are partially or substantially coated with translocation sequences.

Inactivated Viruses, Bacteria, Fungii, and Other Disease Causing Microbes

The disclosure provides compositions comprising an SpHtp1 translocation sequence and a particle coupled to the translocation sequence. In some embodiments the particle is a virus, bacteria, fungus, or other disease causing microbe. Preferably the virus, bacteria, fungii, or other disease causing microbe is inactivated.

The disclosure also provides compositions comprising, consisting, or consisting essentially of a virus, bacteria, fungus, or other disease causing microbe wherein the virus, bacteria, fungus, or other disease causing microbe expresses on its surface a polypeptide comprising an SpHtp1 translocation sequence.

In some preferred embodiments the translocation sequence has at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6.

The term “inactivated” as used herein means that the virus, bacteria, fungus, or other disease causing microbe has undergone treatment to inactivate, kill, or otherwise modify the microbe so as to substantially eliminate its pathogenic properties while retaining its immunogenicity. The inactivated virus, bacteria, fungus, or other disease causing microbe may be any type of disease causing microbe which can express polypeptides on its surface. For example, in microbes comprising a lipid membrane or envelope, transmembrane proteins can be present in the membrane or envelope.

Thus, in some embodiments a polypeptide comprising an SpHtp1 translocation sequence may be present in, or coupled to, an external portion of a transmembrane protein expressed on the surface of the virus, bacteria, fungus, or other disease causing microbe. That is, the translocation sequence is present in, or coupled to, an external portion of a transmembrane protein that is expressed in the membrane or envelope of the virus, bacteria, fungus, or other disease causing microbe.

In such embodiments, polypeptides comprising an SpHtp1 translocation sequence are expressed in the membrane or envelope of the virus, bacteria, fungus, or other disease causing microbe in numbers sufficient to enhance translocation of the microbe across the membrane of a eukaryotic cell. Thus in some embodiments at least 1000, 2000, 3000, 4000, or 5000 polypeptides comprising a translocation sequence are expressed on the surface of the virus, bacteria, fungus, or other disease causing microbe. In some embodiments at least 10,000, 20,000, 30,000, 40,000, or 50,000 polypeptides comprising a translocation sequence are expressed on the surface of the virus, bacteria, fungus, or other disease causing microbe.

Pharmaceutical Compositions

The compositions of the disclosure may be formulated into pharmaceutical compositions comprising any composition or vaccine composition described herein as the active agent (active ingredient), admixed with a pharmaceutically acceptable diluent, excipient or carrier. In some embodiments the pharmaceutical composition may further comprise one or more therapeutic agents or compositions for co-administration with any composition or vaccine composition described herein.

For use according to the present disclosure, the compositions described herein may be presented as a pharmaceutical formulation, comprising the composition or any physiologically acceptable salt, ester or other physiologically functional derivative thereof, together with one or more pharmaceutically acceptable carriers and optionally other therapeutic and/or prophylactic ingredients. The carrier(s) must be acceptable in the sense of being compatible with the other ingredients of the formulation and not deleterious to the recipient thereof. The pharmaceutical compositions may be for human or animal use, in human and veterinary medicine.

Examples of suitable excipients for the various different forms of pharmaceutical compositions described herein may be found in the “Handbook of Pharmaceutical Excipients”, 2nd Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s), buffer(s), flavouring agent(s), surface active agent(s), thickener(s), preservative(s) (including anti-oxidants) and the like, and substances included for the purpose of rendering the formulation isotonic with the blood of the intended recipient.

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p hydroxybenzoic acid. Antioxidants and suspending agents may be also used.

Pharmaceutical formulations include those suitable for oral, topical (including dermal, buccal and sublingual), rectal or parenteral (including subcutaneous, intradermal, intramuscular and intravenous), nasal and pulmonary administration e.g., by inhalation. The formulation may, where appropriate, be conveniently presented in discrete dosage units and may be prepared by any of the methods well known in the art of pharmacy. All methods include the step of bringing into association an active compound with liquid carriers or finely divided solid carriers or both and then, if necessary, shaping the product into the desired formulation.

Pharmaceutical formulations suitable for oral administration wherein the carrier is a solid are most preferably presented as unit dose formulations such as boluses, capsules or tablets each containing a predetermined amount of active agent. A tablet may be made by compression or moulding, optionally with one or more accessory ingredients. Compressed tablets may be prepared by compressing in a suitable machine an active agent in a free-flowing form such as a powder or granules optionally mixed with a binder, lubricant, inert diluent, lubricating agent, surface-active agent or dispersing agent. Moulded tablets may be made by moulding an active agent with an inert liquid diluent. Tablets may be optionally coated and, if uncoated, may optionally be scored. Capsules may be prepared by filling an active agent, either alone or in admixture with one or more accessory ingredients, into the capsule shells and then sealing them in the usual manner. Cachets are analogous to capsules wherein an active agent together with any accessory ingredient(s) is sealed in a rice paper envelope. An active agent may also be formulated as dispersible granules, which may for example be suspended in water before administration, or sprinkled on food. The granules may be packaged, e.g., in a sachet. Formulations suitable for oral administration wherein the carrier is a liquid may be presented as a solution or a suspension in an aqueous or non-aqueous liquid, or as an oil-in-water liquid emulsion.

Formulations for oral administration include controlled release dosage forms, e.g., tablets wherein an active agent is formulated in an appropriate release—controlling matrix, or is coated with a suitable release—controlling film. Such formulations may be particularly convenient for prophylactic use.

Pharmaceutical formulations suitable for rectal administration wherein the carrier is a solid are most preferably presented as unit dose suppositories. Suitable carriers include cocoa butter and other materials commonly used in the art. The suppositories may be conveniently formed by admixture of an active agent with the softened or melted carrier(s) followed by chilling and shaping in moulds.

Pharmaceutical formulations suitable for parenteral administration include sterile solutions or suspensions of an active agent in aqueous or oleaginous vehicles.

Injectable preparations may be adapted for bolus injection or continuous infusion. Such preparations are conveniently presented in unit dose or multi-dose containers which are sealed after introduction of the formulation until required for use. Alternatively, an active agent may be in powder form which is constituted with a suitable vehicle, such as sterile, pyrogen-free water, before use.

An active compound may also be formulated as long-acting depot preparations, which may be administered by intramuscular injection or by implantation, e.g., subcutaneously or intramuscularly. Depot preparations may include, for example, suitable polymeric or hydrophobic materials, or ion-exchange resins. Such long-acting formulations are particularly convenient for prophylactic use.

Formulations suitable for pulmonary administration via the buccal cavity may be presented such that particles containing an active compound and desirably having a diameter in the range of 0.5 to 7 microns are delivered in the bronchial tree of the recipient.

As one possibility such formulations are in the form of finely comminuted powders which may conveniently be presented either in a pierceable capsule, suitably of, for example, gelatin, for use in an inhalation device, or alternatively as a self-propelling formulation comprising an active agent, a suitable liquid or gaseous propellant and optionally other ingredients such as a surfactant and/or a solid diluent. Suitable liquid propellants include propane and the chlorofluorocarbons, and suitable gaseous propellants include carbon dioxide. Self-propelling formulations may also be employed wherein an active agent is dispensed in the form of droplets of solution or suspension.

Such self-propelling formulations are analogous to those known in the art and may be prepared by established procedures. Suitably they are presented in a container provided with either a manually-operable or automatically functioning valve having the desired spray characteristics; advantageously the valve is of a metered type delivering a fixed volume, for example, 25 to 100 microlitres, upon each operation thereof.

As a further possibility an active agent may be in the form of a solution or suspension for use in an atomizer or nebuliser whereby an accelerated airstream or ultrasonic agitation is employed to produce a fine droplet mist for inhalation.

Formulations suitable for nasal administration include preparations generally similar to those described above for pulmonary administration. When dispensed such formulations should desirably have a particle diameter in the range 10 to 200 microns to enable retention in the nasal cavity; this may be achieved by, as appropriate, use of a powder of a suitable particle size or choice of an appropriate valve. Other suitable formulations include coarse powders having a particle diameter in the range 20 to 500 microns, for administration by rapid inhalation through the nasal passage from a container held close up to the nose, and nasal drops comprising 0.2 to 5% w/v of an active agent in aqueous or oily solution or suspension.

Pharmaceutically acceptable carriers are well known to those skilled in the art and include, but are not limited to, 0.1 M and preferably 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Preservatives and other additives may also be present, such as, for example, antimicrobials, antioxidants, chelating agents, inert gases and the like.

Formulations suitable for topical formulation may be provided for example as gels, creams or ointments. Such preparations may be applied e.g. to a wound or ulcer either directly spread upon the surface of the wound or ulcer or carried on a suitable support such as a bandage, gauze, mesh or the like which may be applied to and over the area to be treated.

Liquid or powder formulations may also be provided which can be sprayed or sprinkled directly onto the site to be treated, e.g. a wound or ulcer. Alternatively, a carrier such as a bandage, gauze, mesh or the like can be sprayed or sprinkle with the formulation and then applied to the site to be treated.

According to a further aspect of the disclosure, there is provided a process for the preparation of a pharmaceutical or veterinary composition as described above, the process comprising bringing the active compound(s) into association with the carrier, for example by admixture.

In general, the formulations are prepared by uniformly and intimately bringing into association the active agent with liquid carriers or finely divided solid carriers or both, and then if necessary shaping the product. The disclosure extends to methods for preparing a pharmaceutical composition comprising bringing an agent into association with a pharmaceutically or veterinarily acceptable carrier or vehicle.

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific agent employed, the metabolic stability and length of action of that agent, the age, body weight, general health, sex, diet, mode and time of administration, rate of excretion, drug combination, the severity of the particular condition, and the individual undergoing therapy.

Administration Routes

The pharmaceutical compositions of the present disclosure may be adapted for rectal, nasal, intrabronchial, topical (including buccal and sublingual), vaginal or parenteral (including subcutaneous, intramuscular, intravenous, intraarterial and intradermal), intraperitoneal or intrathecal administration. The pharmaceutical compositions of the present disclosure may be adapted for immersion administration.

Preferably the composition is administered by injection, inhalational, immersion, or oral routes.

Formulations for injection administration may be presented as solutions or emulsions which may be injected intravenously, intraarterially, intrathecally, subcutaneously, intradermally, intraperitoneally or intramuscularly, and which are prepared from sterile or sterilisable solutions.

Formulations for immersion administration are presented as immersion solutions.

Formulations for oral administration may be presented as: discrete units such as capsules, gellules, drops, cachets, pills or tablets each containing a predetermined amount of the active agent; as a powder or granules; as a solution, emulsion or a suspension of the active agent in an aqueous liquid or a non-aqueous liquid; or as an oil-in-water liquid emulsion or a water-in-oil liquid emulsion; or as a bolus etc.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropyl-methylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

The pharmaceutical compositions of the present disclosure may also be in form of suppositories, pessaries, suspensions, emulsions, lotions, ointments, creams, gels, sprays, solutions or dusting powders.

The pharmaceutical compositions of the present disclosure may also be formulated for transdermal administration, for example by use of a cream or skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

Methods and Uses

The present disclosure also provides methods and uses employing the compositions and pharmaceutical compositions of the disclosure described above. Methods of translocating payloads into fish cells, both in vitro and in vivo in live animals, using SpHtp1 translocation sequences have previously been described by the authors (WO 2011/148135 and WO 2014/191759).

While it was previously believed that this effect was specific to fish cells, here the authors surprisingly show that SpHtp1 derived translocation sequences are also able to translocate into mammalian (human) cells, such as HEK cells or the human epithelial lung cell line A549—see Example 2. Previous work had incorrectly indicated that SpHtp1 translocation sequences were not able to direct translocation into mammalian cells (Wawra et al. 2012, PNAS Vol 109 (6) pp 2096-2101; Wawra et al. 2012, MPMI Vol 26 (5) pp 528-36; WO 2014/191759). This surprising discovery opens the possibility of applying the compositions of the disclosure in a much wider variety of animal species than was previously believed possible. This is of particular interest as the authors have previously demonstrated that payloads coupled to SpHtp1 translocation sequences can be delivered into the cells of live animals, in quantities sufficient to yield a cellular immune response (see WO 2014/191759, in which antigenic payloads are delivered into live fish and elicit an immune (antibody) response).

Thus, the present disclosure provides a method of translocating a payload across the membrane of a eukaryotic cell, the method comprising: coupling a translocation sequence to a payload to form a composition of the disclosure; and, contacting the composition with a eukaryotic cell. In some embodiments the method comprises: coupling a translocation sequence to a particle to form a composition of the disclosure; and, contacting the particle with a eukaryotic cell.

The present disclosure also provides a method of delivering a molecule across the membrane of a eukaryotic cell, the method comprising: formulating a molecule into a particle, such that the particle encapsulates or contains the molecule; coupling the particle to a translocation sequence to form a composition of the disclosure; and, contacting the particle with a eukaryotic cell. When formulated into a particle, the molecule may be referred to as a cargo molecule.

The present disclosure also provides for the use of a composition of the disclosure to enhance translocation of a payload across the plasma membrane of a eukaryotic cell, wherein: the composition of the disclosure comprises a translocation sequence coupled to a payload; the membrane of the eukaryotic cell comprises a β-adrenergic receptor or homologue thereof; and, the translocation sequence interacts with the β-adrenergic receptor to enhance translocation of the payload across the membrane. Preferably the β-adrenergic receptor is a β2-adrenergic receptor or homologue thereof.

As described herein, translocating across the membrane of a eukaryotic cell and delivering across the membrane of a eukaryotic cell can mean enhancing or improving the translocation or delivery of a payload or molecule as compared to translocation or delivery of the payload or molecule when it is not coupled to a translocation sequence. As used herein, enhancing can mean an improvement in the rate or maximum amount of payload or molecule that is delivered across the membrane. Translocating across the membrane of a eukaryotic cell and delivering across the membrane of a eukaryotic cell can mean delivery of the payload or molecule into the interior of a cell.

The authors have previously demonstrated enhanced translocation of coupled payloads into eukaryotic cells using mRFP fusion constructs, in which SpHtp1-mRFP was clearly visible in fish cells following incubation of these cells with the fusion construct (WO 2011/148135). Similarly, in WO 2014/191759 the authors demonstrated that antigenic payloads coupled to an SpHtp1 translocation sequence were delivered into cells of live fish in quantities sufficient to elicit an immune (antibody) response. In Example 1, the authors demonstrate SpHtp1-mediated enhancement of the translocation of large particle payloads into cells, using SpHtp1-coupled fluorescent microspheres. The SpHtp1-coupled fluorescent microspheres visibly translocate into the cells following co-incubation.

In the methods of the disclosure the payload, particle, or molecule is delivered into the interior of the cell. In some embodiments the payload or molecule is delivered into vesicles present in the interior of the cell. In some embodiments the payload or molecule is delivered into the cytoplasmic compartment of the cell. In some embodiments the composition of the disclosure is released from cellular vesicles into the cytoplasmic compartment of the cell. In some embodiments the molecule is released from a particle into the cytoplasmic compartment of the cell.

In some embodiments of the methods of the disclosure the composition of the disclosure elicits a response in the cell, for example an immune response, protein expression or downregulation, or other cellular response. In WO 2014/191759 the authors demonstrate that SpHtp1 translocation sequence coupled payloads can be delivered into live fish cells where they elicit an immune (antibody) response.

The authors have found that SpHtp1 translocation sequences bind to the surface of eukaryotic cells in less than 10 minutes, and translocates into fish cells in less than 30 minutes. Thus, in some embodiments of the methods of the disclosure the composition of the disclosure is contacted with the cell for at least 5, 10, 20, 30, 60, 120, or 180 minutes. In embodiments where the payload is a small molecule, polypeptide, or nucleic acid (for example, an antigen, immunogen, or vaccine; therapeutic agent; protective agent; or cytotoxic agent), the composition of the disclosure is preferably contacted with the cell for at least 5 minutes. In embodiments where the payload is a particle, the composition of the disclosure is preferably contacted with the cell for at least 30 minutes. In embodiments where the payload is a nanoparticle, the composition of the disclosure is preferably contacted with the cell for at least 20, 25, 30, 35, 40, or 45 minutes. In embodiments where the payload is a microparticle, the composition of the disclosure is preferably contacted with the cell for at least 50, 55, 60, 65, 70, or 75 minutes. The authors have found that SpHtp1 translocation sequences do not have any immediate cytotoxic effects on cells, and do not alter cell morphology or viability, even with extended incubation periods of up to 24 hours.

In some embodiments of the methods of the disclosure the composition of the disclosure is contacted with the cell at a temperature between 0-40° C. In some embodiments of the methods of the disclosure the composition of the disclosure is contacted with the cell at a temperature between 4-24° C. In some preferred embodiments the composition of the disclosure is contacted with the cell at a temperature of 18° C. In other embodiments of the methods of the disclosure the composition of the disclosure is contacted with the cell at a temperature between 35-40° C. In some preferred embodiments the composition of the disclosure is contacted with the cell at a temperature of 37° C. In some preferred embodiments of the methods of the disclosure the composition of the disclosure is contacted with the cell at or around a temperature typical of the human or animal body.

In some embodiments the cell is an animal cell, for example a fish cell or a mammalian cell. In some preferred embodiments the cell is a human cell, for example an epithelial cell or smooth muscle cell. In some preferred embodiments the cell is a human lung tissue cell.

In some embodiments, the cell is a cell which expresses G-protein coupled receptors (GPCRs). In some embodiments the cells express adrenergic GPCRs, preferably a β-adrenergic receptor or homologue thereof, and more preferably a β2-adrenergic receptor or homologue thereof. In some embodiments the cell is a cell type which has a high level of expression of the β2-adrenergic receptor or a homologue thereof. The cell may be any cell which expresses a β-adrenergic receptor or homologue thereof, preferably a β2-adrenergic receptor or homologue thereof.

In some embodiments the methods of the disclosure are performed in vitro or ex vivo. That is, in embodiments of the methods and uses of the disclosure the cell may be an in vitro or ex vivo cell. In such embodiments the cell may be an isolated cell, a cell in cell culture, or a cell in a tissue.

Medical Methods and Uses

The present disclosure provides methods of treatment of the human or animal body, the method comprising administering to a subject a therapeutically effective amount of a composition of the disclosure as described above.

The term “treatment,” as used herein in the context of treating a condition refers to treatment and therapy, whether of a human or an animal (e.g., in veterinary applications), in which some desired therapeutic effect is achieved. A therapeutic effect may be, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

The term “therapeutically-effective amount” or “effective amount” as used herein refers to an amount of an active compound, or a material, composition or dosage form comprising an active compound, which is effective for producing some desired therapeutic effect, commensurate with a reasonable benefit/risk ratio, when administered in accordance with a desired treatment regimen.

The compositions of the disclosure can be administered by any technique and by any suitable administration route currently utilised in the art. Suitable techniques and administration routes as known in the art are outlined in detail above. In preferred embodiments compositions of the disclosure are administered to a subject by the injection, inhalational, immersion, or oral route.

The compositions of the disclosure can be administered to any human or animal subject in need thereof. In some preferred embodiments the subject is a fish or other gilled animal. In other preferred embodiments the subject is a mammal. In some preferred embodiments the subject is a human or animal which is not a fish. In some particularly preferred embodiments the subject is human.

Subjects suitable for treatment with the compositions of the disclosure include mammals, both human and non-human. Specifically contemplated non-human mammals include livestock, for example pigs, cattle, and sheep, and domesticated animals, for example dogs and cats. Thus, the present disclosure provides methods of treatment of a mammalian subject, the method comprising administering a therapeutically effective amount of a composition of the disclosure. The present disclosure also provides methods of treatment of a human or animal subject wherein the subject is not a fish, the method comprising administering a therapeutically effective amount of a composition of the disclosure. Advantageously, the compositions of the disclosure may be administered to mammals, including humans, by the injection, oral, or inhalational administration routes. The present disclosure therefore provides a method of treatment of a mammalian subject, the method comprising providing a composition of the disclosure; and, administering the composition to the subject by the inhalational administration route.

Other subjects suitable for treatment with compositions of the disclosure include gilled animals such as fish. As used herein, “gilled animals” refers to animals having gills or gill-like respiratory organs, for example fish, molluscs, crustaceans, aquatic insects, and amphibians. Thus, the present disclosure provides methods of treatment of a subject wherein the subject is a gilled animal, the method comprising administering a therapeutically effective amount of a composition of the disclosure. Advantageously, the compositions of the disclosure may be administered without handling the gilled animal (e.g. via immersion or oral routes). The present disclosure therefore provides a method of treatment of a fish or other gilled animal, the method comprising: providing an immersion solution comprising a composition of the disclosure; immersing the fish or other gilled animal in the immersion solution; and, incubating the fish or other gilled animal in the immersion solution for a treatment period.

The methods and compositions of the disclosure can be used to treat any disease or condition known in the art. In some preferred embodiments where the subject is a fish or other gilled animal, the disease is a disease of the gills. In some preferred embodiments where the subject is a human or other mammal, the disease is a lung/respiratory disease. Exemplary lung/respiratory diseases include cystic fibrosis, chronic obstructive pulmonary disease (COPD), asthma, or lung cancer, or bacterial, viral, or fungal lung infection. In some preferred embodiments the disease is an epithelial disease. Exemplary epithelial diseases include acne, eczema, psoriasis, and epithelial carcinoma. In some preferred embodiments the disease is an infectious disease and the composition of the disclosure is a vaccine composition. In some preferred embodiments the disease is a disease of the mouth, stomach, or gut.

The present disclosure also provides compositions of the disclosure as described above for use in a method of treatment of the human or animal body. Embodiments include those in which the method is according to any one of the methods of treatment described herein.

The disclosure also provides compositions of the disclosure as described above for use in the manufacture of a medicament for the treatment of the human or animal body. Embodiments include those in which the treatment is according to any one of the methods of treatment described herein.

Vaccines and Uses

The present disclosure also provides vaccines comprising the compositions of the disclosure, or where appropriate comprising nucleic acids encoding the compositions of the disclosure, and uses of these as vaccines. In particular, the present disclosure envisages the use of the compositions of the disclosure in vaccines against disease in mammals, in particular humans, and in vaccines against disease in fish. The authors have previously demonstrated that antigenic payloads coupled to SpHtp1 translocation sequences can be delivered into the cells of live fish, in quantities sufficient to elicit an immune (antibody) response. In the present application the authors also show that, contrary to what was previously believed, SpHtp1 translocation sequences can also deliver coupled payloads into mammalian (including human) cells via the β2-adrenoceptor.

A vaccine composition is a formulation comprising one or more immunogenic components that is capable of generating a protective immune response in an individual to the one or more immunogenic components. Where the immunogenic components are derived from a pathogen, an individual to whom the vaccine composition has been administered may display acquired and/or adaptive immune responses against the pathogen when subsequently exposed to it. These responses may confer protection against morbidity caused by infection with the pathogen. The vaccine may for example, reduce the likelihood of infection with the pathogen, reduce the severity or duration of the clinical signs of infection in the individual, prevent or delay the onset of clinical signs of infection, or prevent or reduce the risk of the death of the individual following infection with the pathogen.

In some embodiments, the vaccine compositions described herein comprise an SpHtp1 translocation sequence and an antigen or immunogen payload coupled to the translocation sequence. In some embodiments the translocation sequence and coupled antigen is a fusion protein. In some embodiments, the vaccine compositions described herein comprise a nucleic acid encoding such a fusion protein. In other embodiments, the vaccine compositions described herein comprise an expression vector comprising such a nucleic acid.

In some embodiments, the vaccine compositions described herein comprise an SpHtp1 translocation sequence and a particle coupled to the translocation sequence, wherein the particle encapsulates or contains an antigen or immunogen cargo molecule. In some embodiments, the vaccine compositions described herein comprise a translocation sequence and a particle coupled to the translocation sequence, wherein the particle is an inactivated virus, bacteria, fungus, or other disease causing microbe.

In some embodiments, the vaccine compositions described herein comprise an inactivated virus, bacteria, fungus, or other disease causing microbe which expresses on its surface a polypeptide comprising an SpHtp1 translocation sequence.

In some embodiments the vaccine compositions may comprise in addition to the above compositions or composition nucleic acids, a pharmaceutically acceptable excipient, carrier, buffer, or stabiliser (e.g. protease inhibitor) or other materials well known to those skilled in the art. Such materials should be non-toxic and should not interfere with the efficacy of the active ingredient (i.e. the vaccine compositions of the disclosure). The precise nature of the carrier or other material may depend on the route of administration, e.g. inhalational or immersion routes.

In some embodiments the vaccine compositions comprise components that increase the efficacy of uptake of the composition or composition nucleic acid of the disclosure by the organism to be immunized, for example cofactors of a payload.

The present disclosure also provides vaccine compositions of the disclosure for use in inducing an immune response in a human or animal subject. The disclosure also provides vaccine compositions of the disclosure for use in the manufacture of a medicament for inducing an immune response in a human or animal subject.

The disclosure also provides methods of inducing an immune response in a human or animal subject, and methods of reducing the likelihood of contracting a condition associated with infection by a pathogen in a human or animal subject. Such methods comprise administering an immunologically effective dose of a vaccine composition of the disclosure.

In some embodiments the immune response includes the generation of antibodies against one or more antigens comprised in the payload, particle, or cargo molecule or expressed on the surface of the inactivated virus, bacteria, fungus, or other disease causing microbe. In some embodiments the immune response is only the generation of antibodies against one or more antigens comprised in the payload, particle, or cargo molecule or expressed on the surface of the inactivated virus, bacteria, fungus, or other disease causing microbe. In some embodiments the immune response is a reduced likelihood of infection with a pathogen. In some embodiments the immune response is a reduced likelihood of contracting a condition associated with infection by a pathogen. In some embodiments the immune response is reduced severity or duration of the clinical signs of infection, the prevention or delay of the onset of clinical signs of infection, or the prevention or reduction of the risk of the death following infection with a pathogen. Thus, the vaccine compositions of the disclosure can be utilised in a vaccine strategy to induce an immune response in a human or animal subject.

Vaccine compositions of the disclosure may be produced in various forms, depending upon the route of administration. Formulation of compositions of the disclosure as known in the art are outlined in detail above. For example, the vaccine compositions can be made in the form of sterile aqueous solutions or dispersions, suitable for injectable use, or made in lyophilized forms using freeze-drying techniques. Lyophilized vaccine compositions are typically maintained at about 4° C., and can be reconstituted in a stabilizing solution, e.g., saline or HEPES, with or without adjuvant. Vaccine compositions can also be made in the form of suspensions or emulsions, or immersion solutions.

The vaccine compositions can comprise an adjuvant and be administered in an effective amount to a human or animal in order to elicit an immune response. In some preferred embodiments, the compositions are administered without an adjuvant to a human or animal subject.

These vaccine compositions may contain additives suitable for administration via any conventional route of administration. The vaccine compositions may be prepared for administration to subjects in the form of, for example, liquids, immersion solutions, powders, aerosols, tablets, capsules, enteric-coated tablets or capsules, or suppositories. Thus, the vaccine compositions may also be in the form of, but are not limited to, suspensions, solutions, emulsions in oily or aqueous vehicles, pastes, and implantable sustained-release or biodegradable formulations. Compositions for sustained release or implantation may comprise pharmaceutically acceptable polymeric or hydrophobic materials, such as an emulsion, an ion exchange resin, a sparingly soluble polymer, or a sparingly soluble salt.

The vaccine compositions can be administered to a subject by any technique and by any suitable administration route currently utilised in the art. Suitable techniques and administration routes as known in the art are outlined in detail above. For example, the vaccine compositions may be administered by rectal, nasal, intrabronchial, topical (including buccal and sublingual), vaginal or parenteral (including subcutaneous, intramuscular, intravenous, intraarterial and intradermal), intraperitoneal or intrathecal administration. The vaccine compositions may be administered by immersion administration. In preferred embodiments the vaccine compositions are administered to a subject by the injection, inhalational, immersion, or oral route.

Accordingly, the present disclosure provides a method of vaccinating a human or animal subject, comprising administering to a subject a vaccine composition of the disclosure as described above. In some embodiments the subject is a fish. In other embodiments the subject is a mammal. In some embodiments the subject is a human or animal which is not a fish. In some preferred embodiments the subject is human.

Subjects suitable for treatment with the methods of the disclosure include mammals, both human and non-human. Specifically contemplated non-human mammals include livestock, for example pigs, cattle, and sheep, and domesticated animals, for example dogs and cats. A significant advantage of the vaccine compositions and methods of the disclosure when the subject is a mammal is that the vaccine compositions can be administered by the inhalational administration route. Thus, the present disclosure provides methods of vaccinating a human subject, the method comprising: providing a vaccine comprising a vaccine composition of the disclosure; and, administering the vaccine to a human subject by the inhalational administration route.

Other subjects suitable for treatment with the methods of the disclosure include gilled animals such as fish. A significant advantage of the vaccine compositions and methods of the disclosure when the subject is a gilled animal is that the vaccine compositions can be administered without handling the animal (e.g. via immersion or oral routes). Thus, the present disclosure provides methods of vaccinating a fish or other gilled animal, the method comprising: providing an immersion solution comprising a vaccine composition of the disclosure; immersing the fish or other gilled animal in the immersion solution; and, incubating the fish or other gilled animal in the immersion solution for a treatment period.

While it may be desirable to administer the vaccine compositions of the present disclosure by the inhalational or immersion routes, it is also possible and in some cases desirable to administer vaccine compositions of the present disclosure by injection (for example, subcutaneous, intramuscular, intravenous, intraarterial and intradermal injection). The injected vaccines can comprise an adjuvant and be administered in an effective amount to a human or animal (such as a fish or other gilled animal) in order to elicit an immune response. In preferred embodiments, the compositions are administered without an adjuvant to a human or animal (such as a fish or other gilled animal). It is believed that such administration without an adjuvant will decrease the occurrence and/or severity of “local reactions”.

It will be appreciated that appropriate dosages of the vaccine compositions can vary from individual to individual, or population to population, depending on the circumstances. Determining the optimal dosage will generally involve the balancing of the level of therapeutic benefit against any risk or deleterious side effects of the administration. The selected dosage level will depend on a variety of factors including, but not limited to, the route of administration, the time of administration, the rate of excretion of the vaccine composition, other drugs, compounds, and/or materials used in combination, and the species, breed, maturity, sex, weight, condition and general health of the individual. The amount of vaccine composition and route of administration will ultimately be at the discretion of the veterinary surgeon or physician, although generally the dosage will be to achieve serum concentrations of the vaccine composition which are sufficient to produce a beneficial effect without causing substantial harmful or deleterious side-effects.

Suitable dosing regimens are preferably determined taking into account factors well known in the art including age, weight, sex and medical condition of the subject; the route of administration; the desired effect; and the particular composition employed. The vaccine compositions of the disclosure can be used in multi-dose vaccination formats.

The timing of doses depends upon factors well known in the art. After the initial administration, one or more booster doses may subsequently be administered to maintain antibody titers. An example of a dosing regime would be a dose on day 1, a second dose at 1 or 2 months, a third dose at either 3, 4, 5, 6, 7, 8, 9, 10, 11, or 12 months or greater than 12 months, and additional booster doses at distant times as needed.

Examples of the techniques and protocols mentioned above can be found in Remington's Pharmaceutical Sciences, 16th edition, Osol, A. (ed), 1980.

β-Adrenergic Receptors

The authors have previously shown that the host targeting protein SpHtp1 binds to tyrosine-O-sulfate, and that sulfatase treatment of cells strongly decreases the uptake of Saprolegnia effector proteins into those cells (WO 2011/148135). It was therefore believed that these proteins and homologues thereof recognize and bind O-sulfated cell surface molecules as part of the translocation mechanism.

The authors have now identified a G-protein coupled receptor (GPCR) which is believed to mediate translocation of the Saprolegnia effector protein SpHtp1 into fish and human cells. This GPCR belongs to the class of adrenergic receptors—G-protein coupled receptors involved in the transmission of stimuli from the sympathetic nervous system, and which bind endogenous catecholamines as well as exogenously administered drugs. Adrenergic receptors (also termed adrenoceptors) are divided into two main groups, α- and β, with several-subtypes coupled to different G-proteins.

In the present examples the authors demonstrate that the SpHtp1 effector protein binds to the beta-2 (β2) adrenoceptor, and that SpHtp1 is able to enter human cells (such as HEK cells or the human epithelial lung cell line A549) via the human β2-adrenoceptor (which has 63% identity with the fish receptor). Previously, it was believed that SpHtp1 translocation into cells was fish-specific (see WO 2014/191759).

In humans, the β2-adrenoceptor is known to be highly expressed within lung tissue—for example in bronchial smooth muscle cells and bronchial epithelial cells where activation results in bronchodilation. β2-adrenoceptors are also expressed in cardiac myocytes and vascular smooth muscle cells. The compositions of the present disclosure are therefore particularly well suited to administration by inhalational and injection (particularly intravenous) routes.

The β2-adrenoceptor homologue in fish is highly homologous with the mammalian β2-adrenoceptor (Nickerson et al, 2001), thus it is very likely that the β2-adrenoceptor homologue in fish mediates translocation of SpHtp1 into fish cells. Nickerson et al (2001) identified expression of the fish β2-adrenoceptor homologue in the gills of rainbow trout—which is consistent with the finding of the present authors that SpHtp1 coupled payloads are able to elicit immersion vaccination of live fish (WO 2014/191759). The compositions of the present disclosure are therefore particularly well suited to administration by the immersion route.

This new discovery by the authors has implications for new uses of SpHtp1 translocation sequences, as well as new uses of β-adrenergic receptor modulators, as outlined herein.

β-Adrenergic Receptor Modulators

The authors have demonstrated that a GPCR mediates translocation of the SpHtp1 translocation sequence into eukaryotic cells, and that application of an agent which binds this GPCR is able to inhibit translocation of the SpHtp1 translocation sequence into eukaryotic cells in a concentration-dependent manner—see Example 2.

SpHtp1 is an effector protein from the fish pathogenic oomycete Saprolegnia parasitica, secreted by the pathogen in order to establish an infection and/or suppress the immune response in a target host cell. By demonstrating that translocation of this effector protein is mediated by a GPCR, the authors have uncovered a new and advantageous therapeutic application for agents which inhibit binding of effector proteins to this receptor on fish cells

Thus, the present disclosure provides the use of an agent which inhibits binding of an SpHtp1 translocation sequence to GPCR, to inhibit or block translocation of a peptide across the plasma membrane of a eukaryotic cell, wherein the peptide comprises an SpHtp1 translocation sequence. In some embodiments the peptide is an exogenous pathogenic protein or effector protein. In preferred embodiments the peptide comprises a translocation sequence having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6. In some embodiments the cell is a fish or other gilled animal cell, preferably a fish cell. As used herein, “gilled animals” refers to animals having gills or gill-like respiratory organs, for example fish, molluscs, crustaceans, aquatic insects, and amphibians.

The present disclosure also provides a method of preventing infection of fish or other gilled animals by SpHtp1 dependent pathogens, the method comprising administering to a fish or other gilled animal an agent which inhibits binding of an SpHtp1 translocation sequence to a GPCR of a fish or gilled animal cell. The term “SpHtp1 dependent pathogen” as used herein refers to a pathogen which utilises the SpHtp1 effector protein when establishing an infection.

In some preferred embodiments, the SpHtp1 dependent pathogen is a Saprolegnia genus pathogen. In some embodiments, the Saprolegnia genus pathogen is selected from: S. australis, S. ferax, S. diclina, S. delica, S. longicaulis, S. mixta, S. parasitica, S. sporangium, and/or S. variabilis.

Any agent which inhibits or reduces binding of a peptide comprising an SpHtp1 translocation sequence to a GPCR of a fish or gilled animal cell may be used in the uses and methods of the disclosure. In some embodiments the peptide is an exogenous pathogenic protein or effector protein. In preferred embodiments the peptide comprises a translocation sequence having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6. Thus, in some embodiments any agent which inhibits or reduces binding of a peptide comprising a translocation sequence having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6 to a GPCR of a fish or gilled animal cell may be used in the uses and methods of the disclosure.

In some embodiments, the agent is a G-protein coupled receptor binder, or an SpHtp1 translocation sequence binder. In some preferred embodiments, the agent is a β2-adrenoceptor binder, for example a β2-adrenoceptor agonist, antagonist, inverse agonist, or allosteric binder. Exemplary β2-adrenoceptor agonists include salbutamol and levosalbutamol. Exemplary β2-adrenoceptor antagonists include propranolol and carteolol. In some preferred embodiments, the agent is a β2-adrenoceptor modulator, more preferably a β2-adrenoceptor inhibitor.

The authors have demonstrated that pre-incubation of human A549 cells with the β2-adrenoceptor inhibitor SCH-202676 blocks translocation of the SpHtp1 translocation sequence into these cells in a concentration-dependent manner.

In some embodiments, the agent inhibits the binding of the peptide comprising an SpHtp1 translocation sequence to a GPCR of a fish or gilled animal cell. In some embodiments the agent inhibits the binding of the peptide comprising a translocation sequence by steric blockade. In some embodiments, the agent binds to the GPCR on the fish or gilled animal cell. In other embodiments, the agent binds to the peptide comprising a translocation sequence.

In some embodiments the agent inhibits the binding of the peptide comprising an SpHtp1 translocation sequence by reducing expression of a GPCR on a fish or gilled animal cell.

In some embodiments, the agent inhibits translocation of the peptide comprising an SpHtp1 translocation sequence across the membrane of a cell. As used herein, inhibiting translocation can mean a reduction in the rate or maximum amount of peptide comprising a translocation sequence that is translocated across the cell membrane. In Example 2 the authors demonstrate that translocation of an SpHtp1-mRFP fusion construct into cells is inhibited by the β2-adrenoceptor inhibitor SCH-202676 in a concentration-dependent manner. Incubation with SCH-202676 results in a clear reduction in the amount of SpHtp1-mRFP fusion visible inside cells.

The methods of the disclosure can be used to prevent infection of any fish or gilled animal by SpHtp1 dependent pathogens, particularly Saprolegnia genus pathogens. In some preferred embodiments the gilled animal is a fish. In some preferred embodiments the fish is a salmonid, catfish, carp, sea bass, flat fish, or Tilapia. In some particularly preferred embodiments the fish is a: Grass carp (Ctenopharyngodon idella), Silver carp (Hypophthalmichthys molitrix), catla (Cyprinus catla or Gibelion catla), Common Carp (Cyprinus carpio), Bighead carp (Hypophthalmichthys nobilis or Aristichthys nobilis), Crucian carp (Carassius carassius), Nile Tilapia (Oreochromis niloticus), Mozambique Tilapia (Oreochromis mossambicus), Pangas catfish (Pangasius pangasius), Roho (Labeo rohita), Atlantic salmon (Salmo salar), Arctic charr (Salvelinus alpinus), brown trout (Salmo trutta), rainbow trout (Oncorhynchus mykiss), or sea trout (Salmo trutta).

In the methods of the disclosure the agent which inhibits binding of an spHtp1 translocation sequence to a GPCR of a fish or gilled animal cell can be administered by any technique and by any suitable administration route currently utilised in the art. Suitable techniques and administration routes as known in the art are outlined in detail above. In preferred embodiments these agents are administered by the injection, immersion, or oral route.

Thus, the present disclosure provides a method of preventing infection of fish or other gilled animals by SpHtp1 dependent pathogens, for example Saprolegnia genus pathogens, the method comprising: providing an immersion solution comprising an agent which inhibits binding of an SpHtp1 translocation sequence to a GPCR of a fish or gilled animal cell; immersing the fish or other gilled animal in the immersion solution; and, incubating the fish or other gilled animal in the immersion solution for a treatment period.

The present disclosure also provides agents and compositions comprising agents which inhibit binding of an SpHtp1 translocation sequence comprising to a GPCR of a fish or gilled animal cell, for use in a method of preventing infection of fish or other gilled animals by SpHtp1 dependent pathogens.

The present disclosure also provides agents and compositions comprising agents which inhibit binding of an SpHtp1 translocation sequence to a GPCR of a fish or gilled animal cell, for use in the manufacture of a medicament for preventing infection of fish or other gilled animals by SpHtp1 dependent pathogens.

SpHtp1 Enhances Release of the Contents of Endocytic Vesicles into the Cytosol

The present disclosure further provides methods employing, and uses of, an SpHtp1 translocation sequence to enhance the release of the contents of vesicles into the cytoplasmic compartment of a eukaryotic cell. The authors have surprisingly shown that SpHtp1 is able to translocate into eukaryotic cells, release itself from endocytosed vesicles, and then effect the release of other molecules (such as SpHtp3, another S. parasitica effector protein) from endocytosed vesicles. This newly discovered effect is believed to be specific to the SpHtp1 effector protein and is envisaged as being particularly beneficial in effecting the release of, e.g. drug molecules, from endocytosed vesicles. A common problem with drug treatments is that while these can be delivered into cells, they remain trapped inside endocytosed vesicles and are unable to enter the cytoplasmic compartment to fulfil their therapeutic function.

Thus, the present disclosure provides a method of enhancing the release of vesicle contents into the cytoplasmic compartment of a eukaryotic cell, the method comprising contacting the cell with a composition comprising an SpHtp1 translocation sequence. In some embodiments the composition comprises a vesicle release sequence comprising an SpHtp1 translocation sequence. A vesicle release sequence as described herein refers to a sequence which is capable of enhancing the release of vesicle contents into the cytosolic compartment of a eukaryotic cell.

In some embodiments the compositions comprise a polypeptide having at least 20, 30, 40, 50, 60, 70, 75, 80, 85, 90, 95, 98, 99 or 100% sequence identity to the SpHtp1²⁴⁻¹⁹⁸ or SpHtp1a²⁴⁻²²¹ shown in FIG. 6. In some preferred embodiments the compositions comprise a polypeptide having at least 20, 30, 40, 50, 60, 70, 75, 80, 85, 90, 95, 98, 99 or 100% sequence identity to the full-length SpHtp1¹⁻¹⁹⁸ or SpHtp1a¹⁻²²¹ shown in FIG. 6. Thus in some embodiments the vesicle release sequence is a polypeptide having at least 20, 30, 40, 50, 60, 70, 75, 80, 85, 90, 95, 98, 99 or 100% sequence identity to the SpHtp1²⁴⁻¹⁹⁸ or SpHtp1a²⁴⁻²²¹ shown in FIG. 6 or a polypeptide having at least 20, 30, 40, 50, 60, 70, 75, 80, 85, 90, 95, 98, 99 or 100% sequence identity to the full-length SpHtp1¹⁻¹⁹⁸ or SpHtp1a¹⁻²²¹ shown in FIG. 6.

The present disclosure also provides a method of delivering a composition to the cytoplasmic compartment of a eukaryotic cell, the method comprising: contacting the cell with the composition such that the composition enters the cell by endocytosis; and contacting the cell with an SpHtp1 translocation sequence. In some embodiments the composition comprises a polypeptide having at least 20, 30, 40, 50, 60, 70, 75, 80, 85, 90, 95, 98, 99 or 100% sequence identity to the SpHtp1²⁴⁻¹⁹⁸ or SpHtp1a²⁴⁻²²¹ shown in FIG. 6. In some preferred embodiments the composition comprises a polypeptide having at least 20, 30, 40, 50, 60, 70, 75, 80, 85, 90, 95, 98, 99 or 100% sequence identity to the full-length SpHtp1¹⁻¹⁹⁸ or SpHtp1a¹⁻²²¹ shown in FIG. 6.

The present disclosure also provides a composition comprising an SpHtp1 translocation sequence for use in a method of treatment of the human or animal body, wherein the treatment comprises administering the composition to a subject in order to enhance release of vesicle contents into the cytoplasmic compartment of the subject's cells. In some embodiments the composition comprises a polypeptide having at least 20, 30, 40, 50, 60, 70, 75, 80, 85, 90, 95, 98, 99 or 100% sequence identity to the SpHtp1²⁴⁻¹⁹⁸ or SpHtp1a²⁴⁻²²¹ shown in FIG. 6. In some preferred embodiments the composition comprises a polypeptide having at least 20, 30, 40, 50, 60, 70, 75, 80, 85, 90, 95, 98, 99 or 100% sequence identity to the full-length SpHtp1¹⁻¹⁹⁸ or SpHtp1a¹⁻²²¹ shown in FIG. 6.

Also provided is the use of an SpHtp1 translocation sequence to enhance the release of vesicle contents into the cytosolic compartment of a eukaryotic cell. In some embodiments the translocation sequence comprises a polypeptide having at least 20, 30, 40, 50, 60, 70, 75, 80, 85, 90, 95, 98, 99 or 100% sequence identity to the SpHtp1²⁴⁻¹⁹⁸ or SpHtp1a²⁴⁻²²¹ shown in FIG. 6. In some preferred embodiments the translocation sequence comprises a polypeptide having at least 20, 30, 40, 50, 60, 70, 75, 80, 85, 90, 95, 98, 99 or 100% sequence identity to the full-length SpHtp1¹⁻¹⁹⁸ or SpHtp1a¹⁻²²¹ shown in FIG. 6.

As used herein, enhancing the release of vesicle contents into the cytoplasmic compartment can mean enhancing or improving the release of vesicle contents as compared to release when the cell has not been contacted with a composition comprising an SpHtp1 translocation sequence. Enhancing can mean an improvement in the rate or maximum amount of vesicle contents that are released, or may mean an improvement in the number of vesicles which release their contents. Delivering to the cytoplasmic compartment of a cell means translocating a composition across the membrane of a cell, and subsequently enhancing its release from vesicles into the cytoplasmic compartment of the cell.

In Example 3, the authors demonstrate SpHtp1-mediated enhancement of the release of vesicle contents into the cytoplasmic compartment using mRFP fusion constructs. Following incubation of fish RTG-2 cells with a recombinant mRFP fusion construct of the S. parasitica effector protein SpHtp3 the mRFP fusion construct is clearly visible in intracellular vesicles. The SpHtp3-mRFP construct is confined to these vesicles, with very little cytosolic RFP fluorescence detected. However, following pre-incubation with SpHtp1, these vesicles disappear and cytosolic RFP fluorescence is increased, demonstrating involvement of SpHtp1 in the release of SpHtp3-mRFP from the endocytic vesicles.

As used herein, “vesicles” refers to any intracellular vesicles which is present inside a eukaryotic cell. In some preferred embodiments, the vesicles are endocytic vesicles. The endocytic vesicles may be formed by any cellular mechanism, for example by caveolae-mediated endocytosis, clathrin-mediated endocytosis, or lipid raft-mediated endocytosis.

The vesicle contents can be any molecule or material encapsulated or contained within an intracellular vesicle. In some embodiments, the vesicle contents is a composition of the disclosure as described above. In some embodiments, the vesicle contents is a composition comprising an SpHtp1 translocation sequence. In other embodiments, the vesicle contents is a composition which does not comprise an SpHtp1 translocation sequence. In some embodiments, the vesicle contents is a composition co-administered to the cell with the SpHtp1 translocation sequence.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the disclosure in diverse forms thereof.

While the disclosure has been described in conjunction with the exemplary embodiments described above, many equivalent modifications and variations will be apparent to those skilled in the art when given this disclosure. Accordingly, the exemplary embodiments of the disclosure set forth above are considered to be illustrative and not limiting. Various changes to the described embodiments may be made without departing from the spirit and scope of the disclosure.

For the avoidance of any doubt, any theoretical explanations provided herein are provided for the purposes of improving the understanding of a reader. The authors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example+/−10%.

### ASPECTS OF THE DISCLOSURE

The following numbered statements relate to aspects of the present disclosure, and form part of the description:
101. A composition comprising:
   (i) a particle; and
   (ii) an SpHtp1 translocation sequence coupled to the particle.
102. A composition according to statement 101, wherein the particle is a microparticle (0.1-100 μm in size).
103. A composition according to statement 101, wherein the particle is a nanoparticle (1-100 nm in size).
104. A composition according to statement 101, wherein the particle is at least 10, 20, 30, 40, 50, 60, 70, 80, 90 or 100 nm in size.
105. A composition according to statement 101 or 104, wherein the particle is less than 100, 90, 80, 70, 60, 50, 40, 30, 20, or 10 μm in size.
106. A composition according to any one of statements 101 or 104-105, wherein the particle is between 10-10,000, or 20-6000 nm in size.
107. A composition according to any one of statements 100-106, wherein the particle has a rod-shaped, polyhedral, or spherical geometry.
108. A composition according to any one of statements 101-107, wherein the particle:
   (i) is an imaging agent, for example a metallic particle, semiconductor particle (such as a quantum dot), or fluorescent particle;
   (ii) is a magnetic particle, for example Dynabeads®;
   (iii) is a polymeric or liposomal nanoparticle; or
   (iv) has an outer membrane comprising lipid, plastic, or other synthetic or natural polymer.
109. A composition according to any one of statements 101-107, wherein the particle:
   (i) is a virus or viral particle (also called virions); or
   (ii) is a bacteria, fungus, or other disease causing microbe;
   optionally wherein the virus bacteria, fungus, or other disease causing microbe is inactivated.
110. A composition according to statement 109, wherein the virus, bacteria, fungus, or other disease causing microbe expresses the SpHtp1 translocation sequence on its surface.
111. A composition according to any of statements 101-110, wherein the particle encapsulates or contains a cargo molecule.
112. A composition according to statement 111, wherein the cargo molecule is:
   (i) a marker or imaging agent, for example a fluorescent molecule such as FITC or a fluorescent protein such as GFP or RFP;
   (ii) a polypeptide or nucleic acid;
   (iii) an antibody;
   (iv) an antigen, immunogen, or vaccine;
   (v) an antibiotic agent;
   (vi) a lipid;
   (vii) a small organic molecule; or
   (viii) a metal.
113. A composition according to statement 111, wherein the cargo molecule is:
   (i) a therapeutic agent;
   (ii) a protective agent; or
   (iii) a cytotoxic agent.
114. A composition according to any one of statements 101-113, wherein the translocation sequence is bonded to the particle via a covalent, hydrogen, or electrostatic bond, or associated with the particle via hydrophobic association or van der Waals interactions.
115. A composition according to any one of statements 100-114, wherein the translocation sequence is conjugated to the particle through a linker or spacer molecule.
116. A composition according to any one of statements 101-114, wherein the translocation sequence is conjugated directly to the particle.
117. A composition according to any one of statements 101-116, wherein the composition is not a fusion protein.
118. A composition according to any one of statements 101-117, wherein at least 1000, 2000, 3000, 4000, or 5000 translocation sequences are coupled to the particle.
119. A composition according to any one of statements 101-117, comprising a plurality of translocation sequences coupled to the particle, wherein the number of translocation sequences is sufficient to enhance translocation of the composition across the membrane of a eukaryotic cell.
120. A composition according to any one of statements 101-119, wherein the particle is partially or substantially coated with translocation sequences.
121. A composition comprising a virus, bacteria, fungus, or other disease causing microbe; wherein the virus, bacteria, fungus, or other disease causing microbe expresses on its surface a polypeptide comprising an SpHtp1 translocation sequence;
   optionally wherein the virus, bacteria, fungus, or other disease causing microbe is inactivated.
122. A composition according to any one of statements 101-121, wherein the translocation sequence comprises a polypeptide having at least 20, 30, 40, 50, 60, 70, 75, 80, 85, 90, 95, 98, 99 or 100% identity to the amino acid sequence of SpHtp1²⁴⁻¹⁹⁸ or SpHtp1a²⁴⁻²²¹ shown in FIG. 6.
123. A composition according to any one of statements 101-121, wherein the translocation sequence comprises a polypeptide having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6.
124. A composition according to any one of statements 101-123, wherein the translocation sequence enhances translocation of the composition across the membrane of a eukaryotic cell.
125. A composition according to any one of statements 101-124, for use in a method of treatment of the human or animal body.
126. A method of treating a human or animal body, the method comprising administering to a subject a therapeutically effective amount of a composition according to any one of statements 101-124.
127. A composition for use or method according to statement 126 or 127, wherein the composition is administered by injection, inhalational, immersion, or oral routes.
128. A composition for use or method according to any one of statements 125-127, wherein the method is a method of treatment of lung/respiratory disease.
129. A vaccine comprising a composition according to any one of statements 101-124, wherein the particle comprises or encapsulates a target antigen;
   optionally in combination with a pharmaceutically acceptable excipient, carrier, buffer or stabilizer.
130. A vaccine according to statement 129, for inducing an immune response in a human or animal subject.
131. A vaccine according to statement 129, wherein the immune response is the generation of antibodies against the particle or an antigen cargo molecule.
132. A method of vaccinating a human or animal comprising administering to a subject a vaccine according to statement 129.
133. A vaccine for use or method according to any one of statements 130-132, wherein the vaccine is administered by injection, inhalational, immersion, or oral routes.
134. A vaccine for use or method according to any one of statements 130-132, wherein the subject is a fish or a human.
135. A method of vaccinating a human or mammalian subject, the method comprising:
   providing a vaccine comprising a composition according to any one of statements 101-124, wherein the particle comprises or encapsulates a target antigen; and
   administering the vaccine to a human subject by the inhalational administration route.
136. A method of vaccinating a fish or other gilled animal, the method comprising:
   providing an immersion solution comprising a composition according to any one of statements 101-124, wherein the particle comprises or encapsulates a target antigen;
   immersing the fish or other gilled animal in the immersion solution; and
   incubating the fish or other gilled animal in the immersion solution for a treatment period.
137. Use of an SpHtp1 translocation sequence to enhance translocation of a particle across the membrane of a eukaryotic cell; wherein
   the translocation sequence is coupled to the particle;
   the membrane comprises a β-adrenergic receptor or homologue thereof; and
   the translocation sequence interacts with the β-adrenergic receptor to enhance translocation of the nanoparticle across the membrane.
138. A method of translocating a particle across the membrane of a eukaryotic cell, the method comprising:
   coupling an SpHtp1 translocation sequence to a particle; and
   contacting the particle with a eukaryotic cell.
139. A method of delivering a molecule across the membrane of a eukaryotic cell, the method comprising:
   formulating a molecule into a particle, such that the particle encapsulates or contains the molecule;
   coupling the particle to an SpHtp1 translocation sequence; and
   contacting the particle with a eukaryotic cell.
140. A use or method according to any one of statements 137-139, wherein the particle is delivered into the interior of the cell.
141. A use or method according to any one of statements 137-139, wherein the particle is delivered into the cytoplasmic compartment of the cell.
142. A use or method according to any one of statements 137-141, wherein the particle elicits a response in the cell, for example an immune response, protein expression or downregulation, or other cellular response.
143. A method according to any one of statements 138-142, wherein the particle is contacted with the cell for at least 1, 5, 10, 20, 30, 60, 120, or 180 minutes.
144. A method according to any one of statements 138-143, wherein the particle is contacted with the cell at a temperature between 4-24° C.
145. A method according to any one of statements 138-143, wherein the particle is contacted with the cell at a temperature between 35-40° C.
146. A method according to any one of statements 138-145, wherein the cell expresses a β-adrenergic receptor or homologue thereof in its membrane.
147. A use or method according to any one of statements 137-146, wherein the cell is fish cell or a mammalian cell, preferably a human cell.
148. A use or method according to any one of statements 137-147, wherein the cell is an epithelial cell or smooth muscle cell, in particular a human epithelial cell or smooth muscle cell.
149. A use or method according to any one of statements 137-148, wherein the cell is an in vitro or ex vivo cell.
201. A composition for use in a method of treatment of a mammalian subject, the composition comprising:
   (i) an SpHtp1 translocation sequence; and
   (ii) a payload coupled to the translocation sequence.
202. A composition for use in a method of treatment of a human or animal subject, the composition comprising:
   (i) an SpHtp1 translocation sequence; and
   (ii) a payload coupled to the translocation sequence;
   wherein the subject is not a fish.
203. A composition for use according to statement 201 or 202, wherein the subject is human.
204. A composition for use according to any one of statements 201-203, wherein the composition is administered by the injection, inhalational, or oral route.
205. A composition for use according to any one of statements 201-204, wherein the method is a method of treatment of lung/respiratory disease.
206. A composition for use according to any one of statements 201-205, wherein the payload is:
   (i) a marker or imaging agent, for example a fluorescent molecule such as FITC or a fluorescent protein such as GFP or RFP;
   (ii) a polypeptide or nucleic acid;
   (iii) an antibody;
   (iv) an antibiotic agent;
   (v) a lipid;
   (vi) a small organic molecule; or
   (vii) a metal.
207. A composition for use according to any one of statements 201-205, wherein the payload is:
   (i) a therapeutic agent;
   (ii) a protective agent; or
   (iii) a cytotoxic agent.
208. A composition for use according to any one of statements 201-205 or 207, wherein the payload is a therapeutic agent useful in the treatment of lung/respiratory disease.
209. A composition for use according to any one of statements 201-205, wherein the payload is an antigen or immunogen.
210. A composition for use according to any one of statements 201-205, wherein the payload is a particle.
211. A composition for use according to statement 210, wherein the particle is a microparticle (0.1-100 μm in size).
212. A composition for use according to statement 210, wherein the particle is a nanoparticle (1-100 nm in size).
213. A composition for use according to statement 210, wherein the particle is at least 10, 20, 30, 40, 50, 60, 70, 80, 90 or 100 nm in size.
214. A composition for use according to statement 210 or 213, wherein the particle is less than 100, 90, 80, 70, 60, 50, 40, 30, 20, or 10 μm in size.
215. A composition for use according to any one of statements 210 or 213-214, wherein the particle is between 10-10,000, or 20-6000 nm in size.
216. A composition for use according to any one of statements 210-215, wherein the particle has a rod-shaped, polyhedral, or spherical geometry.
217. A composition for use according to any one of statements 210-216, wherein the particle:
   (i) is an imaging agent, for example a metallic particle, semiconductor particle (such as a quantum dot), or fluorescent particle;
   (ii) is a magnetic particle, for example Dynabeads®;
   (iii) is a polymeric or liposomal nanoparticle; or
   (iv) has an outer membrane comprising lipid, plastic, or other synthetic or natural polymer.
218. A composition for use according to any one of statements 210-216, wherein the particle:
   (i) is a virus or viral particle (also called virions); or
   (ii) is a bacteria, fungus, or other disease causing microbe;
   optionally wherein the virus bacteria, fungus, or other disease causing microbe is inactivated.
219. A composition for use according to statement 218, wherein the virus, bacteria, fungus, or other disease causing microbe expresses the translocation sequence on its surface.
220. A composition for use according to any of statements 210-219, wherein the particle encapsulates or contains a cargo molecule.
221. A composition for use according to statement 220, wherein the cargo molecule is:
   (i) a marker or imaging agent, for example a fluorescent molecule such as FITC or a fluorescent protein such as GFP or RFP;
   (ii) a polypeptide or nucleic acid;
   (iii) an antibody;
   (iv) an antigen, immunogen, or vaccine;
   (v) an antibiotic agent;
   (vi) a lipid;
   (vii) a small organic molecule; or
   (viii) a metal.
222. A composition for use according to statement 220, wherein the cargo molecule is:
   (i) a therapeutic agent;
   (ii) a protective agent; or
   (iii) a cytotoxic agent.
223. A composition for use according to any one of statements 206-222, wherein the payload is bonded to the translocation sequence via a covalent, hydrogen, or electrostatic bond, or associated with the translocation sequence via hydrophobic association or van der Waals interactions.
224. A composition for use according to any one of statements 206-222, wherein the payload is conjugated to the translocation sequence through a linker or spacer molecule.
225. A composition for use according to any one of statements 206-222, wherein the payload is conjugated directly to the translocation sequence.
226. A composition for use according to any one of statements 206, 209 or 223-225, wherein the composition is a fusion protein.
227. A composition for use according to any one of statements 201-226, wherein the translocation sequence comprises a polypeptide having at least 20, 30, 40, 50, 60, 70, 75, 80, 85, 90, 95, 98, 99 or 100% identity to the amino acid sequence of SpHtp1²⁴⁻¹⁹⁸ or SpHtp1a²⁴⁻²²¹ shown in FIG. 6.
228. A composition for use according to any one of statements 201-226, wherein the translocation sequence comprises a polypeptide having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6.
229. A composition for use according to any one of statements 201-228, wherein the translocation sequence enhances translocation of the composition across the membrane of a eukaryotic cell.
230. A method of treating a mammalian subject, the method comprising administering to the subject a therapeutically effective amount of a composition comprising:
   (i) an SpHtp1 translocation sequence; and
   (ii) a payload coupled to the translocation sequence.
231. A method of treating a human or animal subject, the method comprising administering to the subject a therapeutically effective amount of a composition comprising:
   (i) an SpHtp1 translocation sequence; and
   (ii) a payload coupled to the translocation sequence;
   wherein the subject is not a fish.
232. A method according to statement 230 or 231, wherein the subject is human.
233. A method according to any one of statements 230-232, wherein the composition is administered by the injection, inhalational, or oral route.
234. A method according to any one of statements 230-233, wherein the method is a method of treatment of lung/respiratory disease.
235. A method according to any one of statements 230-234, wherein the method is a method of vaccinating the subject.
236. A method of vaccinating a human or mammalian subject, the method comprising:
   providing a vaccine comprising:
      (i) an SpHtp1 translocation sequence; and
      (ii) an antigenic or immunogenic payload coupled to the translocation sequence; and administering the vaccine to the subject.
301. A method of preventing infection of fish by SpHtp1 dependent pathogens, the method comprising administering to a fish an agent which inhibits binding of an SpHtp1 translocation sequence to a GPCR of a fish cell.
302. A method according to statement 301, wherein the agent is a G-protein coupled receptor binder or an SpHtp1 translocation sequence binder.
303. A method according to statement 302, wherein the agent is a β-adrenergic receptor modulator, preferably a β2-adrenergic receptor modulator.
304. A method according to statement 303, wherein the agent is a β-adrenergic receptor inhibitor, preferably a β2-adrenergic receptor inhibitor.
305. A method according to any one of statements 301-304, wherein the SpHtp1 dependent pathogen is a Saprolegnia genus pathogen.
306. A method according to statement 305, wherein the SpHtp1 dependent pathogen is selected from: S. australis, S. ferax, S. diclina, S. delica, S. longicaulis, S. mixta, S. parasitica, S. sporangium, and/or S. variabilis.
307. A method according to any one of statements 301-306, wherein the fish is a salmonid, catfish, carp, sea bass, flat fish, or Tilapia.
308. A method according to any statements 307, wherein the fish is a: Grass carp (Ctenopharyngodon idella), Silver carp (Hypophthalmichthys molitrix), catla (Cyprinus catla or Gibelion catla), Common Carp (Cyprinus carpio), Bighead carp (Hypophthalmichthys nobilis or Aristichthys nobilis), Crucian carp (Carassius carassius), Nile Tilapia (Oreochromis niloticus), Mozambique Tilapia (Oreochromis mossambicus), Pangas catfish (Pangasius pangasius), Roho (Labeo rohita), Atlantic salmon (Salmo salar), Arctic charr (Salvelinus alpinus), brown trout (Salmo trutta), rainbow trout (Oncorhynchus mykiss), or sea trout (Salmo trutta).
309. A method according to any one of statements 301-309, wherein the agent is administered by the injection, immersion, or oral route.
310. An agent which inhibits binding of an SpHtp1 translocation sequence to a fish GPCR, for use in a method of preventing infection of fish by SpHtp1 dependent pathogens.
311. Use of an agent which inhibits binding of an SpHtp1 translocation sequence to a GPCR to inhibit or block translocation of a polypeptide across the plasma membrane of a eukaryotic cell, wherein the polypeptide comprises an SpHtp1 translocation sequence.
312. A use according to statement 311, wherein the eukaryotic cell is a fish cell.
313. A use according to any one of statements 311-312, wherein the translocation sequence comprises a polypeptide having at least 20, 30, 40, 50, 60, 70, 75, 80, 85, 90, 95, 98, 99 or 100% identity to the amino acid sequence of SpHtp1²⁴⁻¹⁹⁸ or SpHtp1a²⁴⁻²²¹ shown in FIG. 6.
314. A use according to any one of statements 311-312, wherein the translocation sequence comprises a polypeptide having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6.
315. A use according to any one of statements 311-314, wherein the translocation sequence enhances translocation of the composition across the membrane of a eukaryotic cell.
401. A method of enhancing the release of vesicle contents into the cytoplasmic compartment of a eukaryotic cell, the method comprising contacting the cell with a composition comprising an SpHtp1 translocation sequence.
402. A method of delivering a composition to the cytoplasmic compartment of a eukaryotic cell, the method comprising:
   contacting the cell with the composition such that the composition enters the cell by endocytosis; and
   contacting the cell with an SpHtp1 translocation sequence.
403. A composition comprising an SpHtp1 translocation sequence, for use in a method of treatment of the human or animal body,
   wherein the treatment comprises administering the composition to a subject in order to enhance the release of the contents of endocytic vesicles into the cytosol of a eukaryotic cell.
404. Use of an SpHtp1 translocation sequence to enhance the release of vesicle contents into the cytosolic compartment of a eukaryotic cell.
405. A method, composition for use, or use according to any one of statements 401-404, wherein the translocation sequence comprises a polypeptide having at least 20, 30, 40, 50, 60, 70, 75, 80, 85, 90, 95, 98, 99 or 100% identity to the amino acid sequence of SpHtp1²⁴⁻¹⁹⁸ or SpHtp1a²⁴⁻²²¹ shown in FIG. 6.
406. A method, composition for use, or use according to any one of statements 401-404, wherein the translocation sequence comprises a polypeptide having at least 20, 30, 40, 50, 60, 70, 75, 80, 85, 90, 95, 98, 99 or 100% sequence identity to the full-length SpHtp1¹⁻¹⁹⁸ or SpHtp1a¹⁻²²¹ shown in FIG. 6.
407. A method, composition for use, or use according to any one of statements 401-406, wherein the vesicles are endocytic vesicles.
408. A method, composition for use, or use according to any one of statements 401-407, wherein the vesicle contents is a composition co-administered to the cell with the SpHtp1 translocation sequence.

### Some Embodiments

The following numbered paragraphs relate to some specific embodiments of the present disclosure, and form part of the description:
1. A composition comprising:
   (i) a particle; and
   (ii) a translocation sequence comprising a polypeptide having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6 coupled to the particle.
2. A composition according to paragraph 1, wherein the particle:
   (i) is a polymeric or liposomal nanoparticle;
   (ii) has an outer membrane comprising lipid, plastic, or other synthetic or natural polymer;
   (iii) is a virus or viral particle;
   (iv) is a, fungus, or other disease causing microbe; and/or
   (v) encapsulates or contains a cargo molecule.
3. A composition according to paragraph 2, wherein the cargo molecule is:
   (i) a polypeptide or nucleic acid;
   (ii) an antigen, immunogen, or vaccine;
   (iii) a therapeutic agent;
   (iv) a protective agent; or
   (v) a cytotoxic agent.
4. A composition according to any one of paragraphs 1-4, wherein at least 1000, 2000, 3000, 4000, or 5000 translocation sequences are coupled to the particle.
5. A composition according to any one of paragraphs 1-4, comprising a plurality of translocation sequences comprising a polypeptide having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6 coupled to the particle, wherein the number of translocation sequences is sufficient to enhance translocation of the composition across the membrane of a eukaryotic cell.
6. A composition according to any one of paragraphs 1-5, wherein the particle is partially or substantially coated with translocation sequences.
7. A composition according to any one of paragraphs 1-6, for use in a method of treatment of a human or animal subject.
8. A method of treating a human or animal body, the method comprising administering to a subject a therapeutically effective amount of a composition according to any one of paragraphs 1-26.
9. A composition for use or method according to paragraph 7 or 8, wherein the composition is administered by the injection, inhalational, or immersion route.
10. A composition for use or method according to any one of paragraphs 7-9, wherein the method is a method of treatment of lung/respiratory disease.
11. A composition for use or method according to any one of paragraphs 7-10, wherein the subject is a human or a fish.
12. A vaccine comprising a composition according to any one of paragraphs 1-6, wherein the particle comprises or encapsulates a target antigen;
   optionally in combination with a pharmaceutically acceptable excipient, carrier, buffer or stabilizer.
13. A vaccine composition according to paragraph 12, for use in a method of vaccinating a human or animal subject.
14. A method of vaccinating a human subject, the method comprising:
   providing a vaccine comprising a composition according to paragraph 12; and
   administering the vaccine to a human subject by the inhalational administration route.
15. A method of vaccinating a fish or other gilled animal, the method comprising:
   providing an immersion solution comprising a vaccine comprising a composition according to paragraph 12;
   immersing the fish or other gilled animal in the immersion solution; and
   incubating the fish or other gilled animal in the immersion solution for a treatment period.
16. A composition for use in a method of treatment of a mammalian subject, the composition comprising:
   (i) a translocation sequence comprising a polypeptide having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6; and
   (ii) a payload coupled to the translocation sequence.
17. A composition for use in a method of treatment of a human or animal subject, the composition comprising:
   (i) a translocation sequence comprising a polypeptide having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6; and
   (ii) a payload coupled to the translocation sequence;
   wherein the subject is not a fish.
18. A composition for use according to paragraph 16 or 17, wherein the subject is human.
19. A composition for use according to any one of paragraphs 16 to 18, wherein the composition is administered by injection, inhalational, or oral route.
20. A composition for use according one of paragraphs 16 to 19, wherein the payload is:
   (i) a particle;
   (ii) a polypeptide or nucleic acid;
   (iii) an antigen, immunogen, or vaccine;
   (iv) a therapeutic agent;
   (v) a protective agent; or
   (vi) a cytotoxic agent.
21. A method of treating a mammalian subject, the method comprising administering to a subject a therapeutically effective amount of a composition comprising:
   (i) a translocation sequence comprising a polypeptide having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6; and
   (ii) a payload coupled to the translocation sequence.
22. A method of treating a human or animal subject, the method comprising administering to a subject a therapeutically effective amount of a composition comprising:
   (i) a translocation sequence comprising a polypeptide having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6; and
   (ii) a payload coupled to the translocation sequence;
   wherein subject is not a fish.
23. A method of vaccinating a human subject, the method comprising:
   providing a vaccine comprising a composition comprising:
      (i) a translocation sequence comprising a polypeptide having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6; and
      (ii) a target antigen payload coupled to the translocation sequence; and administering the vaccine to a human subject by the inhalational administration route.
24. A method of preventing infection of fish or other gilled animals by a Saprolegnia genus pathogen, the method comprising administering to a fish or other gilled animal a β2-adrenergic receptor modulator.
25. A β2-adrenergic receptor modulator for use in a method of preventing infection fish or other gilled animals by a Saprolegnia genus pathogen.
26. A method or modulator for use according to any one of paragraphs 24 or 25, wherein the β2-adrenergic receptor modulator is administered by the immersion or oral route.
27. A method or modulator for use according to any one of paragraphs 24 to 27, wherein the fish is a: Grass carp (Ctenopharyngodon idella), Silver carp (Hypophthalmichthys molitrix), catla (Cyprinus catla or Gibelion catla), Common Carp (Cyprinus carpio), Bighead carp (Hypophthalmichthys nobilis or Aristichthys nobilis), Crucian carp (Carassius carassius), Nile Tilapia (Oreochromis niloticus), Mozambique Tilapia (Oreochromis mossambicus), Pangas catfish (Pangasius pangasius), Roho (Labeo rohita), Atlantic salmon (Salmo salar), Arctic charr (Salvelinus alpinus), brown trout (Salmo trutta), rainbow trout (Oncorhynchus mykiss), or sea trout (Salmo trutta).
28. A method of enhancing the release of vesicle contents into the cytoplasmic compartment of a eukaryotic cell, the method comprising contacting the cell with a composition comprising a translocation sequence comprising a polypeptide having at least 20, 30, 40, 50, 60, 70, 75, 80, 85, 90, 95, 98, 99 or 100% sequence identity to the SpHtp1²⁴⁻¹⁹⁸ or SpHtp1a²⁴⁻²²¹ shown in FIG. 6, or a polypeptide having at least 20, 30, 40, 50, 60, 70, 75, 80, 85, 90, 95, 98, 99 or 100% sequence identity to the full-length SpHtp1¹⁻¹⁹⁸ or SpHtp1a¹⁻²²¹ shown in FIG. 6.
29. A method of delivering a composition to the cytoplasmic compartment of a eukaryotic cell, the method comprising
   contacting the cell with the composition such that the composition enters the cell by endocytosis; and
   contacting the cell with a translocation sequence comprising a polypeptide having at least 20, 30, 40, 50, 60, 70, 75, 80, 85, 90, 95, 98, 99 or 100% sequence identity to the SpHtp1²⁴⁻¹⁹⁸ or SpHtp1a²⁴⁻²²¹ shown in FIG. 6, or a polypeptide having at least 20, 30, 40, 50, 60, 70, 75, 80, 85, 90, 95, 98, 99 or 100% sequence identity to the full-length SpHtp1¹⁻¹⁹⁸ or SpHtp1a¹⁻²²¹ shown in FIG. 6.
30. A composition comprising a translocation sequence comprising a polypeptide having at least 20, 30, 40, 50, 60, 70, 75, 80, 85, 90, 95, 98, 99 or 100% sequence identity to the SpHtp1²⁴⁻¹⁹⁸ or SpHtp1a²⁴⁻²²¹ shown in FIG. 6, or a polypeptide having at least 20, 30, 40, 50, 60, 70, 75, 80, 85, 90, 95, 98, 99 or 100% sequence identity to the full-length SpHtp1¹⁻¹⁹⁸ or SpHtp1a¹⁻²²¹ shown in FIG. 6, for use in a method of treatment of the human or animal body, wherein the treatment comprises administering the composition to a subject in order to enhance the release of the contents of endocytic vesicles into the cytosol of a eukaryotic cell.
31. Use of a translocation sequence comprising a polypeptide having at least 20, 30, 40, 50, 60, 70, 75, 80, 85, 90, 95, 98, 99 or 100% sequence identity to the SpHtp1²⁴⁻¹⁹⁸ or SpHtp1a²⁴⁻²²¹ shown in FIG. 6 or a polypeptide having at least 20, 30, 40, 50, 60, 70, 75, 80, 85, 90, 95, 98, 99 or 100% sequence identity to the full-length SpHtp1¹⁻¹⁹⁸ or SpHtp1a¹⁻²²¹ shown in FIG. 6, to enhance the release of vesicle contents into the cytosolic compartment of a eukaryotic cell.
101. A composition comprising:
   (i) a particle; and
   (ii) a translocation sequence comprising a polypeptide having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6 coupled to the particle.
102. A composition according to paragraph 101, wherein the particle is at least 10, 20, 30, 40, 50, 60, 70, 80, 90 or 100 nm in size.
103. A composition according to paragraph 101 or 102, wherein the particle:
   (i) is an imaging agent, for example a metallic, semiconductor, or fluorescent particle;
   (ii) is a magnetic particle, for example Dynabeads®;
   (iii) is a polymeric or liposomal nanoparticle;
   (iv) has an outer membrane comprising lipid, plastic, or other synthetic or natural polymer;
   (v) is a virus or viral particle; or
   (vi) is a bacteria, fungus, or other disease causing microbe.
104. A composition according to any of paragraphs 101-103, wherein the particle encapsulates or contains a cargo molecule.
105. A composition according to paragraph 104, wherein the cargo molecule is:
   (i) a marker or imaging agent;
   (ii) a polypeptide or nucleic acid;
   (iii) an antibody;
   (iv) an antigen, immunogen, or vaccine;
   (v) an antibiotic agent;
   (vi) a lipid;
   (vii) a small organic molecule;
   (viii) a metal;
   (ix) a therapeutic agent;
   (x) a protective agent; or
   (xi) a cytotoxic agent.
106. A composition according to any one of paragraphs 101-105, wherein the translocation sequence is bonded to the particle via a covalent, hydrogen, or electrostatic bond, or is associated with the particle via hydrophobic association or van der Waals interactions.
107. A composition according to any one of paragraphs 101-106, comprising a plurality of translocation sequences comprising a polypeptide having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6 coupled to the particle,
   wherein the number of translocation sequences is sufficient to enhance translocation of the composition across the membrane of a eukaryotic cell.
108. A composition comprising a virus, bacteria, fungus, or other disease causing microbe;
   wherein the virus, bacteria, fungus, or other disease causing microbe expresses on its surface a polypeptide comprising a translocation sequence having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6 coupled to the particle;
   optionally wherein the virus, bacteria, fungus, or other disease causing microbe is inactivated.
109. A composition according to any one of paragraphs 101-108, wherein the translocation sequence comprises a polypeptide having at least 20, 30, 40, 50, 60, 70, 75, 80, 85, 90, 95, 98, 99 or 100% identity to the amino acid sequence of SpHtp1²⁴⁻¹⁹⁸ or SpHtp1a²⁴⁻²²¹ shown in FIG. 6, or a polypeptide having at least 20, 30, 40, 50, 60, 70, 75, 80, 85, 90, 95, 98, 99 or 100% sequence identity to the full-length SpHtp1¹⁻¹⁹⁸ or SpHtp1a¹⁻²²¹ shown in FIG. 6.
110. A composition according to any one of paragraphs 101-109, wherein the translocation sequence enhances translocation of the composition across the membrane of a eukaryotic cell.
111. A composition according to any one of paragraphs 101-110, for use in a method of treatment of the human or animal body;
   optionally wherein the composition is administered by injection, inhalational, immersion, or oral routes.
112. A composition for use according to paragraph 111, wherein the method is a method of treatment of lung/respiratory disease,
   optionally wherein the disease is selected from: lung cancer; bacterial, viral, or fungal lung infection; asthma; chronic obstructive pulmonary disease (COPD); and, cystic fibrosis.
113. A vaccine comprising a composition according to any one of paragraphs 101-110, wherein the particle comprises or encapsulates a target antigen;
   optionally in combination with a pharmaceutically acceptable excipient, carrier, buffer or stabilizer.
114. A vaccine according to paragraph 113, for inducing an immune response in a human or animal subject,
   optionally wherein the immune response is the generation of antibodies against the particle or an antigen cargo molecule.
115. A method of vaccinating a human or animal comprising administering to a subject a vaccine according to paragraph 14;
   optionally wherein the vaccine is administered by injection, inhalational, immersion, or oral routes.
116. A vaccine for use according to paragraph 115, wherein the subject is a fish or a mammal, preferably a human.
117. Use of a translocation sequence comprising a polypeptide having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6 to enhance translocation of a particle across the membrane of a eukaryotic cell; wherein
   the translocation sequence is coupled to the particle;
   the membrane comprises a β-adrenergic receptor or homologue thereof; and
   the translocation sequence interacts with the β-adrenergic receptor to enhance translocation of the nanoparticle across the membrane.
118. A method of translocating a particle across the membrane of a eukaryotic cell, the method comprising:
   coupling a translocation sequence comprising a polypeptide having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6 to a particle; and
   contacting the particle with a eukaryotic cell.
119. A method of delivering a molecule across the membrane of a eukaryotic cell, the method comprising:
   formulating a molecule into a particle, such that the particle encapsulates or contains the molecule;
   coupling the particle to a translocation sequence comprising a polypeptide having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6; and
   contacting the particle with a eukaryotic cell.
120. A use or method according to any one of paragraphs 117-119, wherein the particle:
   (i) is delivered into the interior of the cell;
   (ii) is delivered into the cytoplasmic compartment of the cell;
   (iii) elicits a response in the cell, for example an immune response, protein expression or downregulation, or other cellular response;
   (iv) is contacted with the cell for at least 1, 5, 10, 20, 30, 60, 120, or 180 minutes; and/or
   (v) is contacted with the cell at or around a temperature typical of the human or animal body.
121. A use or method according to any one of paragraphs 117-120, wherein the cell:
   (i) expresses a β-adrenergic receptor or homologue thereof in its membrane, preferably a β2-adrenergic receptor or homologue thereof;
   (ii) is a fish cell or a mammalian cell, preferably a human cell;
   (iii) is an epithelial cell or smooth muscle cell, preferably a human epithelial cell or smooth muscle cell; and/or
   (iv) is an in vitro or ex vivo cell.
122. A composition for use in a method of treatment of a mammalian subject, the composition comprising:
   (i) a translocation sequence comprising a polypeptide having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6; and
   (ii) a payload coupled to the translocation sequence.
123. A composition for use in a method of treatment of a human or animal subject, the composition comprising:
   (i) a translocation sequence comprising a polypeptide having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6; and
   (ii) a payload coupled to the translocation sequence;
   wherein the subject is not a fish.
124. A composition for use according to paragraph 122 or 123, wherein:
   (i) the subject is human;
   (ii) the composition is administered by the injection, inhalational, or oral route; and/or
   (iii) the method is a method of treatment of lung/respiratory disease.
125. A composition for use according to any one of paragraphs 122-124, wherein the payload is:
   (i) a marker or imaging agent;
   (ii) a polypeptide or nucleic acid;
   (iii) an antibody;
   (iv) an antibiotic agent;
   (v) a lipid;
   (vi) a small organic molecule;
   (vii) a metal;
   (viii) a therapeutic agent, preferably a therapeutic agent useful in the treatment of lung/respiratory disease;
   (ix) a protective agent; or
   (x) a cytotoxic agent.
126. A composition for use according to any one of paragraphs 122-125, wherein the payload is an antigen or immunogen.
127. A composition for use according to any one of paragraphs 122-124 or 126, wherein the payload is a particle.
128. A composition for use according to any one of paragraphs 122-127, wherein the payload is bonded to the translocation sequence via a covalent, hydrogen, or electrostatic bond, or is associated with the translocation sequence via hydrophobic association or van der Waals interactions.
129. A composition for use according to any one paragraphs 122-126 or 128, wherein the composition is a fusion protein.
130. A composition for use according to any one of paragraphs 122-129, wherein the translocation sequence enhances translocation of the composition across the membrane of a eukaryotic cell.
131. A method of preventing infection of fish or other gilled animals by a Saprolegnia genus pathogen, the method comprising administering to a fish or other gilled animal a β-adrenergic receptor modulator, preferably a β2-adrenergic receptor modulator.
132. A method according to paragraph 131, wherein the β-adrenergic receptor modulator is a β-adrenergic receptor inhibitor, preferably a β2-adrenergic receptor inhibitor.
133. A method according to paragraph 131 or 132, wherein the Saprolegnia genus pathogen is selected from: S. australis, S. ferax, S. diclina, S. delica, S. longicaulis, S. mixta, S. parasitica, S. sporangium, and/or S. variabilis.
134. A method according to any one of paragraphs 131-133, wherein the fish is a salmonid, catfish, carp, sea bass, flat fish, or Tilapia.
135. A method according to paragraph 134, wherein the fish is a: Grass carp (Ctenopharyngodon idella), Silver carp (Hypophthalmichthys molitrix), catla (Cyprinus catla or Gibelion catla), Common Carp (Cyprinus carpio), Bighead carp (Hypophthalmichthys nobilis or Aristichthys nobilis), Crucian carp (Carassius carassius), Nile Tilapia (Oreochromis niloticus), Mozambique Tilapia (Oreochromis mossambicus), Pangas catfish (Pangasius pangasius), Roho (Labeo rohita), Atlantic salmon (Salmo salar), Arctic charr (Salvelinus alpinus), brown trout (Salmo trutta), rainbow trout (Oncorhynchus mykiss), or sea trout (Salmo trutta).
136. A method according to any one of paragraphs 131-135, wherein the β-adrenergic receptor modulator is administered by the injection, immersion, or oral route.
137. A β-adrenergic receptor modulator for use in a method of preventing infection of fish or other gilled animals by a Saprolegnia genus pathogen.
138. Use of a β-adrenergic receptor modulator to inhibit or block translocation of a polypeptide across the plasma membrane of a eukaryotic cell, wherein the polypeptide comprises a translocation sequence having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6.
139. A method of enhancing the release of vesicle contents into the cytoplasmic compartment of a eukaryotic cell, the method comprising contacting the cell with a composition comprising a polypeptide having at least 20, 30, 40, 50, 60, 70, 75, 80, 85, 90, 95, 98, 99 or 100% sequence identity to the SpHtp1²⁴⁻¹⁹⁸ or SpHtp1a²⁴⁻²²¹ shown in FIG. 6, or the full-length SpHtp1¹⁻¹⁹⁸ or SpHtp1a¹⁻²²¹ shown in FIG. 6.
140. A method of delivering a composition to the cytoplasmic compartment of a eukaryotic cell, the method comprising:
   contacting the cell with the composition such that the composition enters the cell by endocytosis; and
   contacting the cell with a composition comprising a polypeptide having at least 20, 30, 40, 50, 60, 70, 75, 80, 85, 90, 95, 98, 99 or 100% sequence identity to the SpHtp1²⁴⁻¹⁹⁸ or SpHtp1a²⁴⁻²²¹ shown in FIG. 6, or the full-length SpHtp1¹⁻¹⁹⁸ or SpHtp1a¹⁻²²¹ shown in FIG. 6.
141. A composition comprising a translocation sequence comprising a polypeptide having at least 20, 30, 40, 50, 60, 70, 75, 80, 85, 90, 95, 98, 99 or 100% sequence identity to the SpHtp1²⁴⁻¹⁹⁸ or SpHtp1a²⁴⁻²²¹ shown in FIG. 6, or the full-length SpHtp1¹⁻¹⁹⁸ or SpHtp1a¹⁻²²¹ shown in FIG. 6, for use in a method of treatment of the human or animal body,
   wherein the treatment comprises administering the composition to a subject in order to enhance the release of the contents of endocytic vesicles into the cytosol of a eukaryotic cell.
142. Use of a translocation sequence comprising a polypeptide having at least 20, 30, 40, 50, 60, 70, 75, 80, 85, 90, 95, 98, 99 or 100% sequence identity to the SpHtp1²⁴⁻¹⁹⁸ or SpHtp1a²⁴⁻²²¹ shown in FIG. 6, or the full-length SpHtp1¹⁻¹⁹⁸ or SpHtp1a¹⁻²²¹ shown in FIG. 6 to enhance the release of vesicle contents into the cytosolic compartment of a eukaryotic cell.
143. A method, composition for use, or use according to any one of paragraphs 139-142, wherein the vesicles are endocytic vesicles.
144. A method, composition for use, or use according to any one of claims 139-143, wherein the vesicle contents is a composition co-administered to the cell with the composition comprising a polypeptide having at least 20, 30, 40, 50, 60, 70, 75, 80, 85, 90, 95, 98, 99 or 100% sequence identity to the SpHtp1²⁴⁻¹⁹⁸ or SpHtp1a²⁴⁻²²¹ shown in FIG. 6, or the full-length SpHtp1¹⁻¹⁹⁸ or SpHtp1a¹⁻²²¹ shown in FIG. 6.

### EXAMPLES

Materials & Methods

Cell Translocation Studies

Cell culture maintenance of fish RTG-2 (ATCC CCL-55) was done according to the manufacturer's or publisher's instructions. L-15 medium was supplemented with 10% FBS, 100 units/ml Penicillin and 100 μg/ml Streptomycin. Wells containing cover slips with attached cells of 80% confluent were washed 2× with HBSS before translocation experiments.

Cell culture maintenance of human A549 cell, was done according to the manufacturer's or publisher's instructions. DMEM medium was supplemented with 10% FBS, 100 units/ml Penicillin and 100 μg/ml Streptomycin. Wells containing cover slips with attached cells of 80% confluent were washed 2× with HBSS before the translocation experiments.

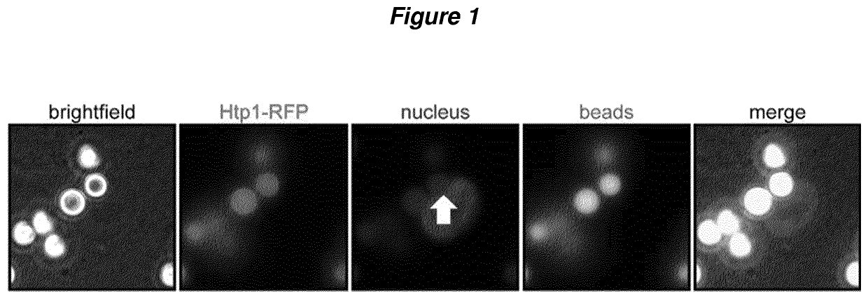

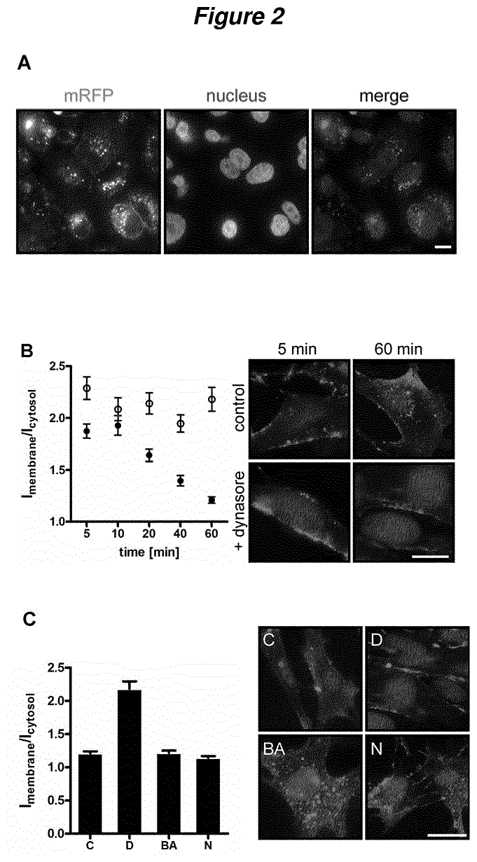

For translocation experiments using SpHtp1-coupled microparticles (FIG. 1), 300 μg carbon beads were pre-incubated with 3 μM SpHtp1²⁴⁻⁶⁸-mRPF in 300 μl DMEM without Fetal Calf Serum for 1 h at 37° C. on a rotator to couple the SpHtp1 translocation sequence to the particles. Coupling of particles and translocation sequence in this way was found to be successful in PBS, Sodium Phosphate buffer (NaPi), and HEPES, and in the presence of milk powder (casein) or BSA. Following coupling, the supplemented medium was applied to the cells for 1 h at 37° C. For translocation experiments using SpHtp1²⁴⁻⁶⁸-mRPF (FIG. 2) cells were incubated with 3 μm SpHtp1²⁴⁻⁶⁸-mRPF for 1 h at 37° C.

For experiments investigating translocation mechanism, cells were pre-incubated with various inhibitors (80 μM of dynasore, 1× Brefeldin A or 100 μM nystatin) for 1 h at 18° C., 2 h for dynasore (FIG. 2). Afterwards, 3 μM SpHtp1²⁴⁻⁶⁸-mRPF were incubated for another 1 h at 18° C. For the time course experiment (FIG. 2) cells were pre-incubated with 10 μM dynasore and the incubation with 3 μM SpHtp1²⁴⁻⁶⁸-mRPF at 18° C. stopped at the times as indicated. In order to investigate the influence of SpHtp1²⁴⁻¹⁹⁸-His on the vesicle release of SpHtp3²¹⁻²¹¹-RFP, 3 μM of each protein was pre-incubated in L-15 for 30 min at RT before added to the cells for the usual incubation of 1 h at 18° C.

For the inhibitor studies, A549 cells were pre-incubated with different concentrations of SCH-202676 (allosteric inhibitor via disulfhydryl group) as indicated for 1 h at 37° C. and after this incubated for another 1 h at 37° C. with 3 μM SpHtp1²⁴⁻⁶⁸-mRPF.

After incubation human as well as fish cells were washed 3× with HBSS and fixed with 4% ice-cold PFA in PBST (PBS+0.1% tween) for 15 min at RT. Residual PFA was removed with 5× washing steps with PBS. Cells were mounted with Vectashield with DAPI. Samples were analysed by a Zeiss LSM710 confocal microscope or an epifluorescence microscope (Zeiss Imager M2 with metal halide light source). For quantification mean intensity or vesicles per cell were counted with ImageJ. A maximum of 5 cells were analysed from the same picture. Bars denote s.e.m of 50 cells counted per sample.

Co-Immunoprecipitation Studies

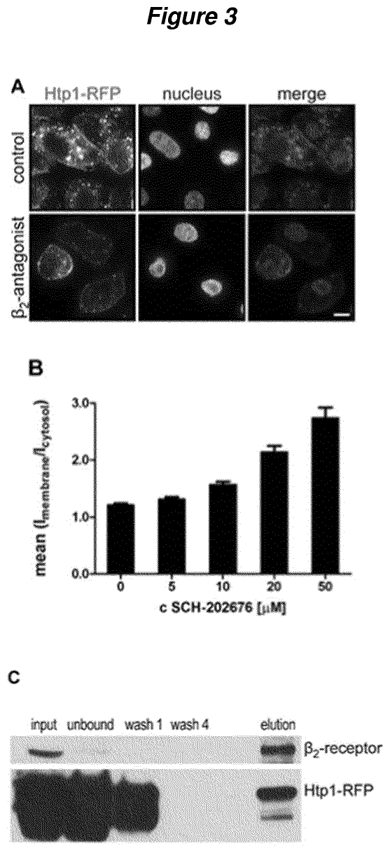

For co-immunoprecipitation (FIG. 3) confluent A549 cells from a 75 cm² flask where detached and resuspended in 500 μl PBS. The cells were lysed by sonication (3×30 sec, 30% intensity, 1 min break on ice) and cell debris removed by centrifugation (16,000×g, 25 min, 4° C.). The supernatant was supplemented with 10 μM SpHtp1²⁴⁻⁶⁸-RFP and 2.5 μg of an anti-82 adrenoceptor and incubated overnight on rotator at 4° C. The next day 200 μl PBS-equilibrated protein G/A beads were added for 2.5 h on rotator at 4° C. After, beads were washed 5 times with 1 ml PBS each. The elution was done with SDS sample buffer and boiling at 95° C. for 10 min.

For the immunoblot, samples were transferred to a nitrocellulose membrane and blocked with 5% milk powder in PBST (PBS+0.1% tween) for 1 h at RT. Incubation of the first antibody (anti-(32 adrenoceptor, 1:200) was done overnight on rotator at 4° C. Excess of antibody was removed by 3 washing steps with PBST, 5 min each. The secondary antibody (anti-rabbit HRP-coupled, 1:1000) was incubated for 1 h at RT in PBST with 5% milk powder. The membrane was washed 4 times (2×PBST and 2×PBS) before the bands were visualised with an ECL substrate and a developer.

Mitochondria Isolation and Effector Binding Studies

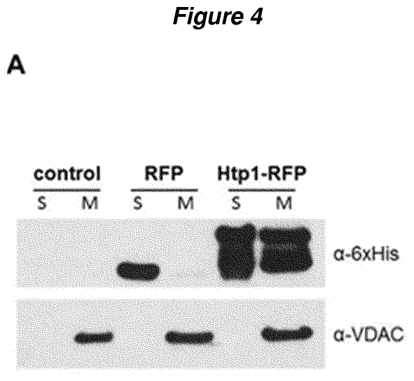

Mitochondria were isolated from confluent RTG-2 cells of a 75 cm² flask (FIG. 4). Cells were detached, harvested and resuspended in 5× of the pellet volume of ice cold isolation buffer (0.3 M mannitol, 0.1% BSA, 0.2 mM EDTA, 10 mM HEPES, pH 7.4). Cells were homogenized with a pestle 5× for 30 sec (250 μl sample volume, 30 sec breaks on ice). Lysate was centrifuged (1000×g, 10 min at 4° C.) and the mitochondria containing supernatant centrifuged again (1000×g for 5 min at 4° C.) to increase purity. To collect the mitochondria the supernatant was centrifuged again (14000×g for 15 min at 4° C.) and the mitochondria containing pellet resuspended in 300 μl isolation buffer. 150 μl mitochondria suspension was incubated with either 50 nM RFP only or SpHtp1²⁴⁻⁶⁸-RFP for 30 min on ice with gently mixing every few minutes. Subsequently, mitochondria were collected (14000×g, 15 min, 4° C.). Samples from the supernatant as well as the pellet were taken for immunoblot analysis.

For the immunoblot, samples were transferred to a nitrocellulose membrane and blocked with 5% milk powder in PBST (PBS+0.1% tween) for 1 h at RT (membrane for the anti-His detection was blocked overnight at 4° C.). Incubation of the first antibody (anti-VDAC, rabbit, 1:200) was done overnight on rotator at 4° C. Excess of antibody was removed by 3 washing steps with PBST, 5 min each. The secondary antibody (anti-goat HRP-coupled, 1:10,000 or anti-His HRP-coupled, 1:10,000) was incubated for 1 h at RT in PBST with 5% milk powder. The membrane was washed 4 times (2×PBST and 2×PBS) before the bands were visualised with an ECL substrate and a developer.

In Vitro Infection Assays with S. parasitica

RTG-2 cells were grown to 70% confluence on glass cover slips. For the infection of RTG-2 cells with S. parasitica 3750 zoospores/cysts were diluted in HBBS supplemented with 10% FBS and 30% L-15 medium. Zoospores/cysts were added to the cells and incubated for 14 h at 24° C.

For fluorescence microscopy cells were washed after co-incubation of cells and spores 3× with HBSS and fixed with 4% ice-cold PFA in PBST (PBS+0.1% tween) for 15 min at RT. Residual PFA was removed with 3× washing steps with PBS and RTG-2 cell/S. parasitica hyphae were stained with SytoRNA for 20 min at RT in the dark. Remaining dye was removed with 3× washing steps with PBS. Successively, membrane was stained by FM4-64FX for 5 min on ice in the dark. Remaining dye was removed with 3× washing steps with PBS. Cells were mounted with Vectashield with DAPI. Samples were analysed by a Zeiss LSM710 confocal microscope.

For experiments investigating vesicular release of SpHtp3, RTG-2 cells were grown to confluence and incubated with 3 μM SpHtp3²¹⁻²¹¹-mRFP for 1 h at 18° C. Cells were washed 3× with L-15 medium and once with HBSS to remove non-translocated protein as well remaining nutrients. Subsequently, 1 ml of zoospores/cysts (3750 cells/nil) of S. parasitica in HBSS supplemented with 3% FBS and 30% L-15 medium were added to the cells. Cells were co-incubated with the zoospores/cysts for another 3 h at 18° C. and SpHtp3-mRFP was monitored by confocal microscopy with a Zeiss LSM 510 confocal microscope equipped with a water dipping lens. Translocation and release from vesicles of SpHtp3²¹⁻²¹¹-mRFP was investigated for 70 min at RT. In total 70 frames were taken, each with a stack of 10 optical slices (z-series) to detect also moving vesicles. Shown are the Z-projections for the time step as indicated which were also used to analyse the decreasing mRFP fluorescence over time with ImageJ. Number of particles was counted for the infected cell compared to all non-infected cells.

Protein Cross Linking

In order to detect a direct interaction, 10 μg of SpHtp1 and 10 μg of SpHtp3 or each protein alone were pre-incubated in PBS (total: 15 μl) for 15 min at RT. For cross linking 15 μl ice-cold 4% PFA/PBS (final concentration: 2%) was added and incubated for 10 min at RT. The reaction was stopped with 7 μl Laemmli loading dye and subsequent heating to 65° C. for 10 min. Complex formation was investigated by SDS-PAGE and confirmed by LC-MS/MS.

### Example 1

SpHtp1 Covered Large Particles can be Translocated into Fish and Human Cells

SpHtp1 is an effector protein that is secreted by the pathogenic oomycete Saprolegnia parasitica during infection of fish to affect the host for the pathogen's benefit (Wawra et al., 2012). The authors have previously shown that the N-terminal moiety of SpHtp1 (24-68 aa) is sufficient for translocation of proteins (recombinant fusion proteins with mRFP or GST) and antigenic payloads into fish cells (WO 2011/148135; WO 2014/191759).

It has now been found that this short residue stretch of SpHtp1 with its self-translocating properties can also be used as a shuttling system to deliver even bigger particles of up to 6 μm in size (for example microbeads and microspheres such as Dynabeads® and Fluoresbrite®) into human cells.

FIG. 1 shows SpHtp1-mediated uptake of Dynabeads® microspheres into human A549 cells. After coating green fluorescent microspheres with SpHtp1 they are able to enter human A549 cells after a 1 h incubation. While a number of beads are still attached to the cell surface, the formation of dents (white arrow head) in some nuclei clearly indicate the uptake of beads with a size of 6 μm.

Because SpHtp1 can be linked to microparticles up to 6 μm in size to transfer these into cells, it has high potential for use as a translocation system for medical compounds that may otherwise be difficult to enter into cells.

### Example 2

Translocation of SpHtp1 into Fish and Human Cells is Mediated by GPCR Receptors

The authors have previously shown that SpHtp1 binds to tyrosine-O-sulfate, and that sulfatase treatment of cells strongly decreases the observed uptake of Saprolegnia host targeting proteins into those cells (Wawra et al., 2012). It is was therefore believed that these proteins and homologs thereof recognize and bind O-sulfated cell surface molecules as part of the translocation mechanism.

Furthermore, the ability of SpHtp1 (and SpHtp1²⁴⁻⁶⁸ linked payloads) to cross cell membranes was previously believed to be fish-specific, as SpHtp1²⁴⁻⁶⁸ containing compositions were shown to be unable to translocate into human (HEK293) or onion cells (see Wawra et al. 2012, PNAS Vol 109 (6) pp 2096-2101) or the human A549 cell line (Wawra et al. 2012, MPMI Vol 26 (5) pp 528-36).

The authors have now identified a GPCR (G-protein coupled receptor) belonging to the class of adrenergic receptors that is responsible for SpHtp1 translocation. Generally, this class of receptors bind endogenous catecholamines and are involved in transmission of stimuli from the sympathetic nervous system. Adrenergic receptors are divided into α- and β-subtypes according to their coupled G-protein.

These experiments indicate that SpHtp1 binds to the β-adrenoceptor, which is well studied in humans and known to mediate smooth muscle relaxation and vasodilation. Furthermore, and contrary to what has been shown previously, the authors have found that SpHtp1 is also able to enter human cells (such as HEK cells or the human epithelial lung cell line A549) via the β-adrenoceptor. The β-adrenoceptor homologue in fish is highly homologous with the mammalian β-adrenoceptor, thus it is very likely that the β-adrenoceptor homologue in fish mediates translocation of SpHtp1 into fish cells.

To investigate the mechanism by which SpHtp1²⁴⁻⁶⁸-mRFP translocates into cells, the fish cell line RTG-2 was incubated with SpHtp1²⁴⁻⁶⁸-mRFP and various compounds known to inhibit different uptake pathways into cells. Brefeldin A (inhibition of caveolae-mediated endocytosis) and nystatin (inhibition of lipid raft-mediated endocytosis) had no effect on SpHtp1²⁴⁻⁶⁸-mRFP translocation, while dynasore (inhibitor of clathrin-mediated endocytosis) resulted in an accumulation of red fluorescence at the membrane, indicating that SpHtp1 is taken up by clathrin-mediated endocytosis (see FIG. 2B).

FIG. 2A shows translocation of SpHtp1²⁴⁻⁶⁸-mRFP into human A549 cells after a 1 h incubation at 37° C., and without 60 mM MgSO4 (used for stabilisation of the protein). Uptake of SpHtp1 is mediated by clathrin-mediated endocytosis and is a time-dependent process. Binding of SpHtp1 to the cell surface occurs within minutes (<5-10 min), with significant uptake into the cell after about 20-30 min (EC50=1.23 μM). Uptake begins as soon as SpHtp1 is bound to the cell surface (see FIG. 2A, 2C).

Further investigation with different inhibitors resulted in the identification of a receptor molecule for SpHtp1 belonging to the group of GPCR's—namely the β-adrenergic receptor. While histamine receptors could be excluded, inhibitors of β-adrenergic receptors inhibit uptake of SpHtp1 but not its binding to the cell (observed as increased fluorescence intensity at the membrane). After pre-incubation of A549 cells with a β2-adrenoceptor inhibitor the overall red fluorescence intensity and the number of vesicles is reduced (FIG. 3A).

Inhibition of SpHtp1 with the β-adrenergic inhibitor SCH-202676 is concentration-dependent (FIG. 3B). In addition, an at least indirect interaction between SpHtp1 and the β2-adrenergic receptor is demonstrated by co-immunoprecipitation (FIG. 3C). SpHtp1 does not interfere with the binding of endogenous ligands of the receptors (acetylcholine or adrenalin/noradrenalin) because these compounds do not have an effect on the uptake of SpHtp1.

Because of a different binding site than the endogenous ligands SpHtp1 does not activate the normal receptor function. Presumably, SpHtp1 binds instead to a distinctive glycosylation pattern of receptor molecules rather than to the protein part of the receptor itself.

These results demonstrate that SpHtp1 is taken up by human, as well as fish cells, via receptor-mediated and clathrin-dependent endocytosis.

### Example 3

SpHtp1 is Involved in Protein Release from Endocytosed Vesicles in Animal and Human Cells

Once inside the cell, SpHtp1 is not confined to vesicles but has access to the cytosol and binds to isolated mitochondria (FIG. 4). The conditions leading to release of SpHtp1 from endocytic vesicles and the cell compartment targeted by SpHtp1 are not fully understood. However, it is known that both the uptake and vesicle release mechanisms of SpHtp1 rely only on the helical integrity of a 44-amino acid stretch (out of 198 amino acids for the full-length protein) that is located N-terminal after the secretion signal peptide (SpHtp1²⁴⁻⁶⁸).

Surprisingly, the authors have shown here that SpHtp1 is also able to effect the release of other proteins, such as SpHtp3 (another effector of S. parasitica) from endocytosed vesicles. SpHtp3, like SpHtp1, is able to translocate into human and animal cells (via a different receptor), where it functions as an RNase.

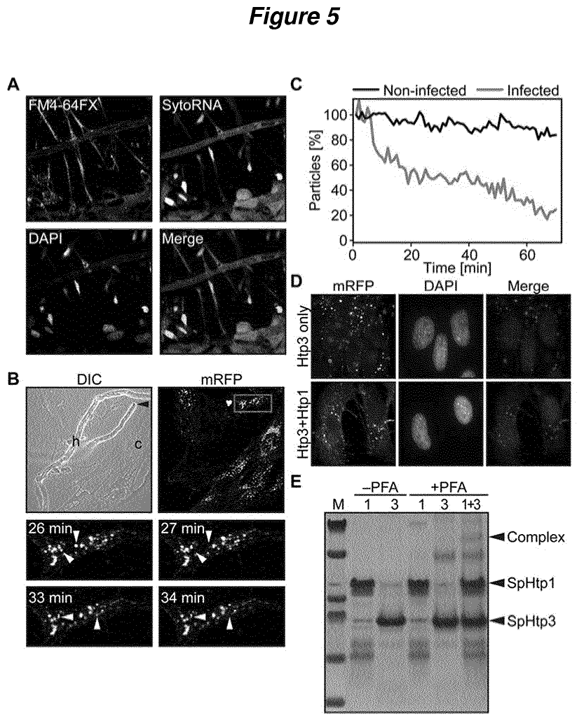

In infection studies of fish RTG-2 cells by S. parasitica degradation of cytoplasmic RNA was observed, as visualised by SytoRNA of cells that are in direct contact to hyphae of S. parasitica while the nuclei of infected cells remain intact (FIG. 5A). Therefore, it was concluded that SpHtp3 or similar, unidentified nucleases must have been translocated into the host cytosol.

To investigate the effect of SpHtp3 under infectious conditions, RTG-2 cells were pre-incubated with recombinant SpHtp3-mRFP. After SpHtp3 was taken up into vesicles, pre-treated cells were co-incubated with S. parasitica. In fish cells that are in direct contact with hyphae of S. parasitica vesicles filled with recombinant SpHtp3-mRFP disappear (FIG. 5B). Remarkably, the number of fluorescent SpHtp3 vesicles was only reduced in cells with direct hyphal contact compared to non-infected cells (75% and 17%, respectively; FIG. 5C). In RTG-2 cells treated with only SpHtp3, this protein remained inside its translocation vesicles—indicating a cofactor-mediated release of SpHtp3.

To investigate a potential interaction between SpHtp1 and SpHtp3, both proteins were pre-incubated before performing the translocation assay into RTG-2 cells. At pH 7.5 the translocation of SpHtp3 into vesicles is significantly reduced—reflected by the low amount of vesicles with a low fluorescence intensity (FIG. 5D).

Following pre-incubation with SpHtp1, vesicles in the periphery of the cell disappear and cytosolic RFP fluorescence is increased. This indicates that SpHtp1 is involved in the uptake and release of SpHtp3 from vesicles at a neutral pH (pH 7.5; FIG. 5D).

Co-incubation of recombinant SpHtp1 and SpHtp3 (mRFP- or His-tagged) in vitro, resulted in an additional band for a cross-linked SpHtp1-SpHtp3 complex (FIG. 5E), which was confirmed by LC-MSMS analysis.

Analysis of the additional band from SDS-PAGE after crosslink is shown in Table 1. SpHtp1-His6 and SpHtp3-His6 were co-incubated and cross linked with 4% PFA/PBS. An additional band appeared which contained peptides for both proteins according to LC-MS/MS analysis.

TABLE 1

| | | | | # Unique | # | # | # | MW |
|---|---|---|---|---|---|---|---|---|
| Accession | Description | Score | Coverage | Peptides | Peptides | PSMs | AAs | [kDa] |
| SPRG_03573T0 | SpHtp3 | 1550.47 | 70.62 | 22 | 22 | 65 | 211 | 23.8 |
| SPRG_04986T0 | SpHtp1 | 135.09 | 24.00 | 4 | 4 | 8 | 200 | 21.6 |
| SPRG_07885T0 | histone H4 | 28.19 | 21.78 | 2 | 2 | 2 | 101 | 11.3 |
| SPRG_15039T0 | unknown | 27.13 | 1.80 | 1 | 1 | 1 | 724 | 79.7 |
| SPRG_14283T0 | unknown | 23.68 | 1.60 | 1 | 1 | 1 | 501 | 55.8 |
| SPRG_04290T0 | unknown | 20.96 | 0.66 | 1 | 1 | 1 | 1213 | 133.3 |

### REFERENCES

A number of publications are cited above in order to more fully describe the disclosure and the state of the art to which the disclosure pertains. Full citations for these references are provided below. The entirety of each of these references is incorporated herein.
WO 2011/148135;
WO 2014/191759;
Wawra et al. 2012, PNAS Vol 109 (6) pp 2096-2101;
Wawra et al. 2012, MPMI Vol 26 (5) pp 528-36;
Sprengel et al, 2017, Nat Commun. Vol 16 (8) pp 4472;
Trusch et al, 2016, Chem Commun (Camb). Vol 52 (98) pp 14141-14144.
Nickerson et al, 2001, Eur J Biochem., 268(24):6465-72
Pearson & Lipman, 1988. Methods in Enzymology 183: 63-98;
A Wade & P J Weller, 1994, Handbook of Pharmaceutical Excipients, 2nd Edition;
Osol, 1980, Remington's Pharmaceutical Sciences;
Gennaro, 1985, Remington's Pharmaceutical Sciences;
For standard molecular biology techniques, see Sambrook, J., Russel, D. W. Molecular Cloning, A Laboratory Manual. 3 ed. 2001, Cold Spring Harbor, N.Y.: Cold Spring Harbor Laboratory Press

1. A composition comprising:
(i) a particle; and
(ii) a translocation sequence comprising a polypeptide having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6 coupled to the particle.

2. A composition according to claim 1, wherein the particle is at least 10, 20, 30, 40, 50, 60, 70, 80, 90 or 100 nm in size.

3. A composition according to claim 1 or 2, wherein the particle:
(i) is an imaging agent, for example a metallic, semiconductor, or fluorescent particle;
(ii) is a magnetic particle, for example Dynabeads®;
(iii) is a polymeric or liposomal nanoparticle;
(iv) has an outer membrane comprising lipid, plastic, or other synthetic or natural polymer;
(v) is a virus or viral particle; or
(vi) is a bacteria, fungus, or other disease causing microbe.

4. A composition according to any of claims 1-3, wherein the particle encapsulates or contains a cargo molecule.

5. A composition according to claim 4, wherein the cargo molecule is:
(i) a marker or imaging agent;
(ii) a polypeptide or nucleic acid;
(iii) an antibody;
(iv) an antigen, immunogen, or vaccine;
(v) an antibiotic agent;
(vi) a lipid;
(vii) a small organic molecule;
(viii) a metal;
(ix) a therapeutic agent;
(x) a protective agent; or
(xi) a cytotoxic agent.

6. A composition according to any one of claims 1-5, wherein the translocation sequence is bonded to the particle via a covalent, hydrogen, or electrostatic bond, or is associated with the particle via hydrophobic association or van der Waals interactions.

7. A composition according to any one of claims 1-6, comprising a plurality of translocation sequences comprising a polypeptide having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6 coupled to the particle,
wherein the number of translocation sequences is sufficient to enhance translocation of the composition across the membrane of a eukaryotic cell.

8. A composition comprising a virus, bacteria, fungus, or other disease causing microbe;
wherein the virus, bacteria, fungus, or other disease causing microbe expresses on its surface a polypeptide comprising a translocation sequence having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6 coupled to the particle;
optionally wherein the virus, bacteria, fungus, or other disease causing microbe is inactivated.

9. A composition according to any one of claims 1-8, wherein the translocation sequence comprises a polypeptide having at least 20, 30, 40, 50, 60, 70, 75, 80, 85, 90, 95, 98, 99 or 100% identity to the amino acid sequence of SpHtp1²⁴⁻¹⁹⁸ or SpHtp1a²⁴⁻²²¹ shown in FIG. 6, or a polypeptide having at least 20, 30, 40, 50, 60, 70, 75, 80, 85, 90, 95, 98, 99 or 100% sequence identity to the full-length SpHtp1¹⁻¹⁹⁸ or SpHtp1a¹⁻²²¹ shown in FIG. 6.

10. A composition according to any one of claims 1-9, wherein the translocation sequence enhances translocation of the composition across the membrane of a eukaryotic cell.

11. A composition according to any one of claims 1-10, for use in a method of treatment of the human or animal body;
optionally wherein the composition is administered by injection, inhalational, immersion, or oral routes.

12. A composition for use according to claim 11, wherein the method is a method of treatment of lung/respiratory disease,
optionally wherein the disease is selected from: lung cancer; bacterial, viral, or fungal lung infection; asthma; chronic obstructive pulmonary disease (COPD); and, cystic fibrosis.

13. A vaccine comprising a composition according to any one of claims 1-10, wherein the particle comprises or encapsulates a target antigen;
optionally in combination with a pharmaceutically acceptable excipient, carrier, buffer or stabilizer.

14. A vaccine according to claim 13, for inducing an immune response in a human or animal subject,
optionally wherein the immune response is the generation of antibodies against the particle or an antigen cargo molecule.

15. A method of vaccinating a human or animal comprising administering to a subject a vaccine according to claim 14;
optionally wherein the vaccine is administered by injection, inhalational, immersion, or oral routes.

16. A vaccine for use according to claim 15, wherein the subject is a fish or a mammal, preferably a human.

17. Use of a translocation sequence comprising a polypeptide having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6 to enhance translocation of a particle across the membrane of a eukaryotic cell; wherein
the translocation sequence is coupled to the particle;
the membrane comprises a β-adrenergic receptor or homologue thereof; and
the translocation sequence interacts with the β-adrenergic receptor to enhance translocation of the particle across the membrane.

18. A method of translocating a particle across the membrane of a eukaryotic cell, the method comprising:
coupling a translocation sequence comprising a polypeptide having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6 to a particle; and
contacting the particle with a eukaryotic cell.

19. A method of delivering a molecule across the membrane of a eukaryotic cell, the method comprising:
formulating a molecule into a particle, such that the particle encapsulates or contains the molecule;
coupling the particle to a translocation sequence comprising a polypeptide having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6; and
contacting the particle with a eukaryotic cell.

20. A use or method according to any one of claims 17-19, wherein the particle:
(i) is delivered into the interior of the cell;
(ii) is delivered into the cytoplasmic compartment of the cell;
(iii) elicits a response in the cell, for example an immune response, protein expression or downregulation, or other cellular response;
(iv) is contacted with the cell for at least 1, 5, 10, 20, 30, 60, 120, or 180 minutes; and/or
(v) is contacted with the cell at or around a temperature typical of the human or animal body.

21. A use or method according to any one of claims 17-20, wherein the cell:
(i) expresses a β-adrenergic receptor or homologue thereof in its membrane, preferably a β2-adrenergic receptor or homologue thereof;
(ii) is a fish cell or a mammalian cell, preferably a human cell;
(iii) is an epithelial cell or smooth muscle cell, preferably a human epithelial cell or smooth muscle cell; and/or
(iv) is an in vitro or ex vivo cell.

22. A composition for use in a method of treatment of a mammalian subject, the composition comprising:
(i) a translocation sequence comprising a polypeptide having at least 60, 70, 75, 80, 85, 90, 95, 98, 99 or 100% sequence identity to the SpHtp1²⁴⁻⁶⁸ or SpHtp1a²⁴⁻⁶⁹ shown in FIG. 6; and
(ii) a payload coupled to the translocation sequence.

23. A composition for use in a method of treatment of a human or animal subject, the composition comprising:
(i) a translocation sequence comprising a polypeptide having at least 60, 70, 75, 80, 85, 90, 95, 98, 99 or 100% sequence identity to the SpHtp124-68 or SpHtp1a24-69 shown in FIG. 6; and
(ii) a payload coupled to the translocation sequence;
wherein the subject is not a fish.

24. A β-adrenergic receptor modulator for use in a method of preventing infection of fish or other gilled animals by a Saprolegnia genus pathogen.

25. A method of enhancing the release of vesicle contents into the cytoplasmic compartment of a eukaryotic cell, the method comprising contacting the cell with a composition comprising a polypeptide having at least 20, 30, 40, 50, 60, 70, 75, 80, 85, 90, 95, 98, 99 or 100% sequence identity to the SpHtp1²⁴⁻¹⁹⁸ or SpHtp1a²⁴⁻²²¹ shown in FIG. 6, or a polypeptide having at least 20, 30, 40, 50, 60, 70, 75, 80, 85, 90, 95, 98, 99 or 100% sequence identity to the full-length SpHtp1¹⁻¹⁹⁸ or SpHtp1a¹⁻²²¹ shown in FIG. 6.


SEQUENCE LISTING (37 sequences)

SEQ ID NO: 1 - protein, 198 aa, Artificial Sequence
  SpHtp1 (1-198)
MRIHHPLTLAALCVVLHESLGAAQHSNNVARLEHYRIAEIEHWEKRHLRSDSRGHRHHAHHGQVIDKENNNSQEQATTGNSVETNQVPSTEPTKDKTTPMKNALFKLFREKKLKTKNAGNGHAHDDDDDSDFSDDDVPTNAPTDAPTGAPTDAPTDAPTVAPTDAPTDAPTEAPTNAPTGTDAPTDAPTDAQVVPTFD

SEQ ID NO: 2 - protein, 175 aa, Artificial Sequence
  SpHtp1 (24-198)
QHSNNVARLEHYRIAEIEHWEKRHLRSDSRGHRHHAHHGQVIDKENNNSQEQATTGNSVETNQVPSTEPTKDKTTPMKNALFKLFREKKLKTKNAGNGHAHDDDDDSDFSDDDVPTNAPTDAPTGAPTDAPTDAPTVAPTDAPTDAPTEAPTNAPTGTDAPTDAPTDAQVVPTFD

SEQ ID NO: 3 - protein, 45 aa, Artificial Sequence
  SpHtp1 (24-68)
QHSNNVARLEHYRIAEIEHWEKRHLRSDSRGHRHHAHHGQVIDKE

SEQ ID NO: 4 - protein, 130 aa, Artificial Sequence
  SpHtp1 (69-198)
NNNSQEQATTGNSVETNQVPSTEPTKDKTTPMKNALFKLFREKKLKTKNAGNGHAHDDDDDSDFSDDDVPTNAPTDAPTGAPTDAPTDAPTVAPTDAPTDAPTEAPTNAPTGTDAPTDAPTDAQVVPTFD

SEQ ID NO: 5 - protein, 221 aa, Artificial Sequence
  SpHtp1a (1-221)
MRIYHPLTLAALCVVLHESLGAAQHSNNVARLEHYSVAEIEHWEKRHLRNSDSRGHRHHAHHGQVIDKENNNSQEQATTGNSVETNQVPFTQPTKDKTASIKTAVFKLLRENKLKTKNAGNGHAHDDDDDDDSNFSDDDVPTNAPTGAPTGAPTDAPTNAPTDAPTDAPTDAPTDAPTDAPTDAPTDAPTTLPPMPRRRSHRCPDRHSHRCDPRRTHGCPD

SEQ ID NO: 6 - protein, 198 aa, Artificial Sequence
  SpHtp1a (24-221)
QHSNNVARLEHYSVAEIEHWEKRHLRNSDSRGHRHHAHHGQVIDKENNNSQEQATTGNSVETNQVPFTQPTKDKTASIKTAVFKLLRENKLKTKNAGNGHAHDDDDDDDSNFSDDDVPTNAPTGAPTGAPTDAPTNAPTDAPTDAPTDAPTDAPTDAPTDAPTDAPTTLPPMPRRRSHRCPDRHSHRCDPRRTHGCPD

SEQ ID NO: 7 - protein, 46 aa, Artificial Sequence
  SpHtp1a (25-69)
QHSNNVARLEHYSVAEIEHWEKRHLRNSDSRGHRHHAHHGQVIDKE

SEQ ID NO: 8 - protein, 152 aa, Artificial Sequence
  HpHtp1a (70-221)
NNNSQEQATTGNSVETNQVPFTQPTKDKTASIKTAVFKLLRENKLKTKNAGNGHAHDDDDDDDSNFSDDDVPTNAPTGAPTGAPTDAPTNAPTDAPTDAPTDAPTDAPTDAPTDAPTDAPTTLPPMPRRRSHRCPDRHSHRCDPRRTHGCPD

SEQ ID NO: 9 - protein, 260 aa, Artificial Sequence
  VP2, partial [Infectious pancreatic necrosis virus]
MLPENGPASIPDDITERHILKQETSSYNLEVSDSGSGLLVCFPGAPGSRVGAHYRWNLNQTELEFDRWLETSQDLKKAFNYGRLISRKYDIQSSTLPAGLYALNGTLNAATFEGSLSEVESLTYNSLMSLTTNPQDKVNNQLVTKGITVLNLPTGFDKPYVRLEDETPQGSQSMNGARMRCTAAIAPRRYEIDLPSERLPTVAATGTPTTIYEGNADIVNSTTVTGDVTFQLAAEPANETRFDFILQFLGLDNDVPVVSV

SEQ ID NO: 10 - protein, 260 aa, Artificial Sequence
  VP2, partial [Infectious pancreatic necrosis virus]
MLPETGPASIPDDITERHILKQETSSYNLEVSESGSGLLVCFPGAPGSRIGAHYRWNANQTGLEFDQWLETSQDLKKAFNYGRLISRKYDIQSSTLPAGLYALNGTLNAATFEGSLSEVESLTYNSLMSLTTNPQDKVNNQLVTKGVTVLNLPTGFDKPYVRLEDETPQGLQSMNGAKMRCTAATAPRRYEIDLPSQRLPPVPATGTLTTLYEGNADIVNSTTVTGDINFSLAEQPAVETKFDFQLDFMGLDNHVPVVTV

SEQ ID NO: 11 - protein, 260 aa, Artificial Sequence
  VP2, partial [Infectious pancreatic necrosis virus]
MLPETGPASIPDDITERHILKQETSSYNLEVSESGSGILVCFPGAPGSRIGAHYRWNANQTGLEFDQWLETSQDLKKAFNYGRLISRKYDIQSSTLPAGLYALNGTLNAATFEGSLSEVESLTYNSLMSLTTNPQDKVNNQLVTKGVTVLNLPTGFDKPYVRLEDETPQGLQSMNGAKMRCTAAIAPRRYEIDLPSQRLPPVPATGALTTLYEGNADIVNSTTVTGDINFSLAEQPAVETKFDFQLDFMGLDNDVPVVTV

SEQ ID NO: 12 - protein, 260 aa, Artificial Sequence; identical to SEQ ID NO: 9
  Infectious pancreatic necrosis virus

SEQ ID NO: 13 - protein, 260 aa, Artificial Sequence; identical to SEQ ID NO: 10
  Infectious pancreatic necrosis virus

SEQ ID NO: 14 - protein, 260 aa, Artificial Sequence; identical to SEQ ID NO: 11
  Infectious pancreatic necrosis virus

SEQ ID NO: 15 - DNA, 1122 bp, Artificial Sequence
  Infectious salmon anemia virus strain VT11052007-27 hemagglutinin-esterase (HE) mRNA, partial cds
atggcacgattcataattttattcctactgttggcgcctgtttacagtcgtctatgtcttagaaactaccctgacaccacctggttaggtgactctcgaagcgatcagtccagagtgaatccacagtctttggatctggtgactgagttcaagggggtgctgcaggccaaaaacggaaatggacttttgaagcagatgagtggaaggtttccaagtgactggtacacacctactacaaagtaccggatcctatacttgggaaccaatgactgcactgacggacctactgacatgatcatcccaacttcgatgacactggacaacgcggcaagggagctgtacctgggagcatgcaggggagacgtgagagtgacgcctacatttgtgggagcagcaattgttggacttgttggacgaacagacgcaattaccggtttttcggtgaaggtgttgactttcaacagccctacaattgtagtggtgggattgaatggaatgtccggaatctacaaggtctgcattgcagcaacatctgggaatgtgggaggagtgacacttatcaacggatgcggatatttcaacacacctttgaggtttgacaatttccaaggacaaatctacgtgtcagacacctttgaagtgaggggaaccaaaaacaagtgtgttctgctaagatcttctagtgatacgcctttgtgttcacacatcatgaggaacgttgagttagatgagtatgtagacacaccaaatacagggggtgtttatccttctgatggttttgattcactacatggttcagcttccgttagaacgttcctcactgatgcattgacatgcccagacattgactggagtagaattgatgctgcttcgtgtgaatatgacagctgccctaagatggttaaagattttgaccagacaagcttaggtaacacagacacacttatcatgagggaggtagcattgcacaaggagatgatcagtaaacttcagaggaacatcacagatgtaaaaacatctgtgttgagcaacatcttcatttctatgggtgtagcaggttttgggattgctctgttcctagcaggttggaaggcatgtatttggattgcagcattcatgtac

SEQ ID NO: 16 - DNA, 1186 bp, Artificial Sequence
  Infectious salmon anemia virus isolate Biovac32719-108 hemagglutinin-esterase protein (HE) mRNA, complete cds
atggcacgattcataattttattcctactgttggcgcctgtttacagtcgtctatgtcttagaaactaccctgacaccacctggttaggtgactctcgaagcgatcagtccagagtgaatccacagtctttggatctggtgactgagttcaagggggtgctgcaggccaaaaacggaaatggacttttgaagcagatgagtggaaggtttccaagtgactggtacacacctactacaaagtaccggatcctatacttgggaaccaatgactgcactgacggacctactgacatgatcatcccaactccgatgacactggacaacgcggcaagggagctgtacctgggagcatgcaggggagacgtgagagtgacgcctacatttgtgggagcagcaattgttggacttgttggacgaacagacgcaattaccggtttttcggtgaaggtgttgactttcaacagctctacaattgtagtggtgggattgaatggaatgtccggaatctacaaggtctgcattgcagcaacatctgggaatgtgggaggagtgacacttatcaacggatgcggatatttcaacacacctttgaggtttgacaatttccaaggacaaatctacgtgtcagacacctttgaagtgaggggaaccaaaaacaagtgtgttctgctaagatcttctagtgatacgcctttgtgttcacacatcatgaggaacgttgagttagatgagtatgtagacacaccaaatacagggggtgtttatccttctgatggttttgattcactacatggttcagcttccgttagaacgttcctcactgatgcattgacatgcccagacattgactggagtagaattgatgctgcttcgtgtgaatatgacagctgccctaagatggttaaagattttgaccagacaagcttaggtaacacagacacacttatcatgagggaggtagcattgcacaaggagatgatcagtaaacttcagaggaacatcacagatgtaaaaacatctgtgttgagcaacatcttcatttctatgggtgtagcaggttttgggattgctctgttcctagcaggttggaaggcatgtatttggattgcagcattcatgtacaagtctagaggtagaattccaccatcgaacctgtttgttgcttgattatgaaagaaagacaacc

SEQ ID NO: 17 - protein, 374 aa, Artificial Sequence
  Salmon isavirus
MARFIILFLLLAPVYSRLCLRNYPDTTWLGDSRSDQSRVNPQSLDLVTEFKGVLQAKNGNGLLKQMSGRFPSDWYTPTTKYRILYLGTNDCTDGPTDMIIPTSMTLDNAARELYLGACRGDVRVTPTFVGAAIVGLVGRTDAITGFSVKVLTFNSPTIVVVGLNGMSGIYKVCIAATSGNVGGVTLINGCGYFNTPLRFDNFQGQIYVSDTFEVRGTKNKCVLLRSSSDTPLCSHIMRNVELDEYVDTPNTGGVYPSDGFDSLHGSASVRTFLTDALTCPDIDWSRIDAASCEYDSCPKMVKDFDQTSLGNTDTLIMREVALHKEMISKLQRNITDVKTSVLSNIFISMGVAGFGIALFLAGWKACIWIAAFMY

SEQ ID NO: 18 - protein, 388 aa, Artificial Sequence
  Salmon isavirus
MARFIILFLLLAPVYSRLCLRNYPDTTWLGDSRSDQSRVNPQSLDLVTEFKGVLQAKNGNGLLKQMSGRFPSDWYTPTTKYRILYLGTNDCTDGPTDMIIPTPMTLDNAARELYLGACRGDVRVTPTFVGAAIVGLVGRTDAITGFSVKVLTFNSSTIVVVGLNGMSGIYKVCIAATSGNVGGVTLINGCGYFNTPLRFDNFQGQIYVSDTFEVRGTKNKCVLLRSSSDTPLCSHIMRNVELDEYVDTPNTGGVYPSDGFDSLHGSASVRTFLTDALTCPDIDWSRIDAASCEYDSCPKMVKDFDQTSLGNTDTLIMREVALHKEMISKLQRNITDVKTSVLSNIFISMGVAGFGIALFLAGWKACIWIAAFMYKSRGRIPPSNLFVA

SEQ ID NO: 19 - DNA, 5178 bp, Artificial Sequence
  Salmon Pancreas Disease virus mRNA for Capsid protein, E3 protein, E2 protein, 6K protein, E1 protein and partial nsP4 protein, isolate F93125
actatggactcagcggcaatgaacgtggaggcttttaaaagtttcgcctgtaaggacaccgacctgtggactgagttcgcggaaaaaccagtaaggttgtcgcccggccaaatcgaagagtatgtctttcatctacaaggggccaaggccaatgtgatgcacagcagagtcgaagccgtatgccctgacctctcggaggtggctatggacaggttcacactagacatgaaacgcgacgtcaaagtgacgccaggcacgaagcacgtagaggagagacctaaagtccaagagattcaagcggccgaccccatggccaccgcgtacttgtgcgccatccatagagagctagtccgaaggctgaaggccgtcctgaaaccgtctatacacgtgttgttcgatatgagctccgaggattttgatgctatcgtgggccatgggatgaagttgggtgacaaggtgctggaaacggacatctcctcattcgacaagagccaggaccaagccatggcggttacagcgctgatgctgctgagggacttgggagtagaagaagacctcctgaccctaattgaggcgtctttcggcgacatcacttctgcccacctgcccacaggcaccagatttcagtttggatcgatgatgaagtctggactttttctgacgctgttcgtgaacacgctgcttaacatcaccatagctgcccgagttttacgggagcagctggctgataccaggtgtgccgcgtttatcggtgacgacaacgtaatcaccggagtagtgtctgacgacatgatggtggccaggtgcgcatcctggctgaacatggaggtgaagatcatggacatggaaattggcaacatgagtccttatttttgtggcggcttcctgttactcgacacggtaacaggcactgtaagccgagtgtcggaccctgtaaaacgcctgatgaagatgggaaaaccggccctgaacgatccagaaacggacgtggacagatgccgcgcactgcgcgaagaagtggaaagctggtacagagtggggattcagtggccactgcaggtggctgccgccacacgctatggcgtgaaccacctgccgctggccacaatggcgatggccacgctcgcccaggacttgagatcgtacctgggcgcgcgaggggagtacgtatccctctacgtctaaccttaatattttctgcatcatacttccaaacaatcatgtttcccatgcaattcaccaactcagcctatcgccagatggagcccatgtttgcaccgggttcccgaggacaagtacagccgtaccggccgcgcactaagcgccgccaggagccgcaagtcggcaacgccgccattactgccctcgcgaaccagatgagtgcgctccagttgcaggtagctggacttgccggccaggcaagggtggaccgccgtgggccaagacgtgttcagaagaacaagcagaagaagaagaactcttccaacggagaaaaacccaaagagaagaagaagaagcaaaaacaacaggagaagaagggaagcggtggcgaaaaagtcaagaagactaggaaccgacccgggaaggaggtaaggatctccgtaaagtgtgcccgacagagcaccttccccgtgtaccacgaaggtgctatatccggctacgctgtgctgattggatctcgcgtattcaagccggcacacgtgaagggtaagatcgaccaccctgaactggcagacatcaagttccaggtcgccgaggacatggacctcgaagcagctgcgtacccgaagagcatgcgagaccaagcggctgaaccagcgaccatgatggacagagtgtacaactgggagtatggcactatcagagtggaggataatgtcataatcgacgcaagcggtaggggcaagccgggtgacagtggcagggccatcaccgacaactcgggaaaggttgttggtattgtcctcggaggaggacccgatggcaggcgcacacgcctctccgtgataggtttcgacaagaagatgaaggctagggagatcgcctacagtgatgccataccttggacacgcgctccggccctcctgctgctgcctatggttattgtctgcacctacaattccaacaccttcgattgctccaaaccgtcctgccaggactgctgcattactgctgaaccagagaaggccatgaccatgctgaaggacaatctgaacgacccgaactactgggacctactcattgctgtcaccacctgtggctccgcccggagaaagagggctgtgtctacgtcgcctgccgccttttacgacacacagatcctcgccgcccacgcagctgcctccccatacagggcgtactgccccgattgtgacggaacagcgtgtatctcgccgatagccatcgacgaggtggtgagcagtggcagcgaccacgtcctccgcatgcgggttggttctcaatcgggagtgaccgctaagggtggtgcggcgggtgagacctctctgcgatacctgggaagggacgggaaggttcacgccgcagacaacacgcgactcgtggtgcgcacgactgcaaagtgcgacgtgctgcaggccactggccactacatcctggccaactgcccagtggggcagagcctaaccgttgcggccacactggatggcacccggcatcaatgcaccacggttttcgaacaccaagtaacggagaagttcaccagagaacgcagcaagggccaccatctgtccgacatgaccaagaaatgcaccagattttccactacaccaaaaaagtccgccctctacctcgttgatgtgtatgacgctctgccgatttctgtagagattagcaccgtcgtaacatgcagcgacagccagtgcacagtgagggtgccacctggtaccacagtgaaattcgacaagaaatgcaagagcgctgactcggcaaccgtcactttcaccagcgactcccagacgtttacgtgtgaggagccagtcctaacggctgccagtatcacccagggcaagccacacctcagatcggcaatgttgcctagcggaggcaaggaagtgaaagcaaggatcccgttcccgttcccgccggaaaccgcaacttgcagagtgagtgtagccccactgccgtcgatcacctacgaggaaagcgatgtcctgctagccggtaccgcaaaataccctgtgctgctaaccacacggaaccttggtttccatagcaacgccacatccgaatggatccagggcaagtacctgcgccgcatcccggtcacgcctcaagggatcgagctaacatggggaaacaacgcgccgatgcacttttggtcatccgtcaggtacgcatccggggacgctgatgcgtacccctgggaacttctggtgtaccacaccaagcaccatccagagtacgcgtgggcgtttgtaggagttgcatgcggcctgctggctatcgcagcgtgcatgtttgcgtgcgcatgcagcagggtgcggtactctctggtcgccaacacgttcaactcgaacccaccaccattgaccgcactgactgcagcactgtgttgcataccaggggctcgcgcggaccaaccctacttggacatcattgcctactttttaggggtaagagggtggtcagccctgctggtcatccttgcgtatgtacagagctgcaagagctacgaacacaccgtggtggtcccaatggatccaagagccccgtcgtacgaagcagtgataaaccggaatgggtatgatccattgaagctgaccatctcagtgaatttcaccgtcatctcaccaactacggctctggaatattggacctgcgcaggagtccccatcgtcgagccgccccatgtgggctgctgcacgtcggtgtcctgcccctctgacctctctacgctgcatgcgtttactggcaaagctgtctccgacgtgcactgcgatgtgcacacaaacgtgtaccccttgttgtggggcgcggctcactgcttctgttccaccgagaatacacaggtcagcgctgtggcagccaccgtttctgagttctgtgcccaggactcagagcgtgccgaagcgttcagcgtacacagcagctcagtcaccgctgaggtcctggtgacgcttggtgaagtggtgacggcagtccacgtttacgtggacggggtaacatcagccaggggcactgacctcaagatcgtggctggaccaataacaaccgactactccccattcgatcgcaaagtagtccgcatcggcgaagaggtctataactatgactggcctccttacggggctggccgaccaggcacattcggagacattcaagctaggtcaaccaactatgtcaaacccaacgatctgtatggggacatcggaattgaagtactgcagccgactaacgaccacgtacatgtggcttacacgtatacgacctctgggttactgcgttggctgcaggacgctccgaaaccactcagtgtcacagcaccgcacggttgtaagatcagtgccaatccgctcctggccctcgattgtggggttggtgccgtccccatgtccatcaacattccggacgcgaagtttacccgcaaattaaaggatccgaaaccatcggccctgaaatgcgtggtggacagctgcgagtacggggtggactacgggggcgccgccacgatcacctacgagggccacgaggccgggaagtgcgggattcattccctgacaccaggagtccccctgagaacatcggtggttgaagtggttgctggcgccaataccgtcaaaacgaccttctcctcacccacgcccgaggttgcactcgaggtagagatctgttcggcaatagtgaagtgcgctggtgagtgcactccaccgaaggaacatgtggtcgcaaccaggcctcgccatggcagcgaccctggaggctacatctccgggcccgcaatgcgctgggccggagggattgtagggaccctagtggtcctgttccttatccttgccgtcatctactgcgtggtgaagaagtgccgctccaaaagaatccggatagtcaagagctaaattccggtatacaaattgctcactaggagcccatccgatcccacagggagtaggatgagtcatctattggttttaaaattttcaatacaaaaaaaaaaaaaaa

SEQ ID NO: 20 - protein, 394 aa, Artificial Sequence
  NsP4
TMDSAAMNVEAFKSFACKDTDLWTEFAEKPVRLSPGQIEEYVFHLQGAKANVMHSRVEAVCPDLSEVAMDRFTLDMKRDVKVTPGTKHVEERPKVQEIQAADPMATAYLCAIHRELVRRLKAVLKPSIHVLFDMSSEDFDAIVGHGMKLGDKVLETDISSFDKSQDQAMAVTALMLLRDLGVEEDLLTLIEASFGDITSAHLPTGTRFQFGSMMKSGLFLTLFVNTLLNITIAARVLREQLADTRCAAFIGDDNVITGVVSDDMMVARCASWLNMEVKIMDMEIGNMSPYFCGGFLLLDTVTGTVSRVSDPVKRLMKMGKPALNDPETDVDRCRALREEVESWYRVGIQWPLQVAAATRYGVNHLPLATMAMATLAQDLRSYLGARGEYVSLYV

SEQ ID NO: 21 - DNA, 4179 bp, Artificial Sequence
  Sleeping disease virus mRNA for polyprotein
cacaatggcgatggccacgctcgcccaggatctgagatcgtacttgggcgcgcgaggggagtacgtatccctctacgcctaaccttaatattttctgcatcatactctcaaccaaccatgtttcccatgcaattcaccaactcagcctatcgccagatggagcccatgttcgcaccggcttctcgaggacaagtacagccgtatcggccgcgcacaaagcgccgccaagagccgcaagtcggcaacgctgctattgctgccctcgcgaaccagatgagcgcgctccagctgcaggtggctggacttgccggccaggcaaggttggaccgtcctggaccgagactttttccagaaaaaacaagcagaagaagaagaactcttccaacggagaaaaacccccggagaagaagaaagaagccaaaaccacctggagaagaaagggaacggcggggaaaaagccaagaaacccccggaaccggccccggaaagaagttaggatctccgttaagcgtgcccgacagaacaccttccccgttgtaccatgacggtgccatatccggctatgcggtgctgattggctcccgcgtgtttaagccagcgcacgtgaagggtaagttcgaccaccccgaactggcggacatcaagttccaggtcgccgaggtcatggacctcgaagcagccgcataccctaagtgcatgcgagaccaggcggctgaaccagcaaccatgatggatggagtgtacaatggggagtacggcaatattcaggagtggaggacaattttgtattcgatgcgagcggcagaggcaagccggggtgacagtggcaggccattcaccgacaactcaggaaaggttgtcggtatcgtcctcggaggaggacccgatggtaggcgcacacgtctctccgtgataggtttcgacaagaagctgaaggccagagagatcgcctacagcgaggccatcccttggacacgcgcaccagctctcctgctgctgcctatggtcatcgcctgcacctacaactccaatacctttgattgctccaaaccgtcctgccaggattgttgcattactgctgaaccaaagaaggccatgactatgctgaaggacaacctgaatgacccgaactactgggacctgctcattgccgtcaccacctgcagttccgcccgaaaaaagagggctgtgtctacgtcgcctgtcgccgtttacgacacacaaattctcgccgcccacgcagctgcctccccgtatagggcgtactgccccgattgtgacggaactgcctgcatctcgccgatagctatcgacgaggtggtaagtagcggtagtgaccacgtccttcgcatccgggtcggttctcaatcgggagtgaccgctaaaggcggtgcggcgggtgaaacctctctgcgatacctgggaagggacggtaaggtttacgccgcggacaacacgcggctcgtggtgcgcaccactgcaaagtgtgacgtgctgcaggccactggccactacattctggccaactgcccagtggggcagagtctcactgttgcggccacactggacggtacccggcatcaatgcaccacggttttcgaacatcaagtaacggagaagttcacaagagaacgcagcaagggccaccacctgtccgatctgaccaagaaatgcaccaggttctccaccaccccgaagaagtccgcgctctatctcgttgatgtgtatgatgctctgccgacttctgtagagatcagcaccgtggtgacatgcaacgaaagacagtgcacagtgagggtgccacccggtaccacagtgaaattcgataagaggtgcaagaacgctgccaaagagaccgtcaccttcaccagcgactcccagacgtttacgtgcgaggagccggtcctaacggccgccagcatcacccagggcaagccgcacctcagatcgtcaatgttgcccagcggaggcaaagaggtgaaagcgaggattccattcccgttcccgccagagactgcgacttgcagagtgagcatcgccccactgccatcgattacctatgaggaaagcgatgttctgctggccggcactgcgaaataccccgtgctgctaactacacggaaccttggtttccatagcaacgccacatctgaatggatccagggtaagtacctgcgccgcatcccggtcacgccccaagggattgaactaatgttgggaaacaacgcaccgctgcacttctggtcatctgtcaggtacgcatctggagacgccgacgcgtacccctgggaacttctggtgcaccacatcaagcaccatccggagtacgcgtgggcgtttgtaggagttgcatgtggcctgctggccgttgcagcatgcatgttcgcgtgcgcatgcaacagggtgcggtactctctgctcgccaacacgttcaacccgaacccaccaccattgaccgcactgactgcagcattgtgctgcatacctggggctcgcgcggatcaaccctacctggacatcattgcctacttgtggaccaacagcaaagtggccttcgggctgcaatgcgcggcgcccgtggcttgcatgctcatcgttacatacgcccttagacattgcagattgtgctgcaattcttttttaggggtaagagggtggtcggctctgctggtcatccttgcgtatgtacagagctgcaaggcgtacgaacacaccgtggtggtcccaatggatccaagagccccgtcgtacgaggcggtgataaaccggaatgggtatgaccccctgaagcttaccatcgcagtgaactttaccgtcatctcaccaactacggctctggaatactggacctgtgcaggagtccctgtcgtcgagccgccccatgtgggctgctgcacgtcagtgtcctgcccctccgacctctccacgctgcacgcgttcaccggcaaagccgtctccgacgtgcactgcgatgtgcacacgaacgtgtaccccttgttgtggggtgcggctcactgcttctgttccactgaaaacacgcaggtcagcgctgtggccgccaccgtttctgagttctgtgctcaggactcagagcgcgccgaggcgttcagcgttcacagcagctcagtcactgcagagattctggtgacgcttggtgaagtggtgacggcggtccacgtttacgtggacggggtaacatcagccaggggtaccgacctcaagatcgtggctggcccaataacaactgactactccccgtttgaccgcaaagtagtccgtatcggcgaagaggtctataattacgactggcctccttacggggctggtcgaccaggcacattcggagacattcaagctaggtcaaccaactatgtcaaacccaatgatctgtacggggacatcggaattgaagtactgcagccgactaatgaccacgtgcacgtggcttacacgtatacgacctctgggttgctgcgttggttgcaggacgctccgaaaccactcagtgtcacagcaccgcacggttgtaagatcagtgctaacccgctcctggccctcgattgtggggttggtgccgtccccatgtccatcaacattccggacgcgaagttcacccgcaaactaaaggacccgaaaccttcggccctgaaatgcgtggtggacagttgcgagtacggggtggactacgggggcgccgccacgatcacctacgagggccacgaggctgggaagtgcgggatccattccctgacaccaggagtccctctgagaacatcagtggttgaagtagttgccggcgctaataccgtcaaaacgaccttctcctcacccacgcccgaggttacactcgaggtagagatctgttcggcaatagtgaagtgcgccagtgagtgcactccaccgaaggaacacgtagtcgcagccaggcctcgccatggcagcgacactggaggctacatctccgggcccgcaatgcgctgggccggaaggattgtagggaaccctagtggtcctgtttcctcatccttggccgtcacctactgcgtggtgaagaagtgccgctctaaaagaatccggatagtcaagagctaaattccggtatacaaatcgctcactaggagcccatccgaacccacagggagtaggatgagtcatctattggtttttaaaattttcaat

SEQ ID NO: 22 - protein, 1322 aa, Artificial Sequence
  Sleeping disease virus
MFPMQFTNSAYRQMEPMFAPASRGQVQPYRPRTKRRQEPQVGNAAIAALANQMSALQLQVAGLAGQARVDRRGPRRVQKNKQKKKNSSNGEKPKEKKKKQKQQEKKGSGGEKAKKPRNRPGKEVRISVKRARQSTFPVYHDGAISGYAVLIGSRVFKPAHVKGKFDHPELADIKFQVAEVMDLEAAAYPKCMRDQAAEPATMMDGVYNGEYGNIQEWRTILYSMRAAEASRGDSGRPFTDNSGKVVGIVLGGGPDGRRTRLSVIGFDKKLKAREIAYSEAIPWTRAPALLLLPMVIACTYNSNTFDCSKPSCQDCCITAEPKKAMTMLKDNLNDPNYWDLLIAVTTCSSARKKRAVSTSPVAVYDTQILAAHAAASPYRAYCPDCDGTACISPIAIDEVVSSGSDHVLRIRVGSQSGVTAKGGAAGETSLRYLGRDGKVYAADNTRLVVRTTAKCDVLQATGHYILANCPVGQSLTVAATLDGTRHQCTTVFEHQVTEKFTRERSKGHHLSDLTKKCTRFSTTPKKSALYLVDVYDALPTSVEISTVVTCNERQCTVRVPPGTTVKFDKRCKNAAKETVTFTSDSQTFTCEEPVLTAASITQGKPHLRSSMLPSGGKEVKARIPFPFPPETATCRVSIAPLPSITYEESDVLLAGTAKYPVLLTTRNLGFHSNATSEWIQGKYLRRIPVTPQGIELMLGNNAPLHFWSSVRYASGDADAYPWELLVHHIKHHPEYAWAFVGVACGLLAVAACMFACACNRVRYSLLANTFNPNPPPLTALTAALCCIPGARADQPYLDIIAYLWTNSKVAFGLQCAAPVACMLIVTYALRHCRLCCNSFLGVRGWSALLVILAYVQSCKAYEHTVVVPMDPRAPSYEAVINRNGYDPLKLTIAVNFTVISPTTALEYWTCAGVPVVEPPHVGCCTSVSCPSDLSTLHAFTGKAVSDVHCDVHTNVYPLLWGAAHCFCSTENTQVSAVAATVSEFCAQDSERAEAFSVHSSSVTAEILVTLGEVVTAVHVYVDGVTSARGTDLKIVAGPITTDYSPFDRKVVRIGEEVYNYDWPPYGAGRPGTFGDIQARSTNYVKPNDLYGDIGIEVLQPTNDHVHVAYTYTTSGLLRWLQDAPKPLSVTAPHGCKISANPLLALDCGVGAVPMSINIPDAKFTRKLKDPKPSALKCVVDSCEYGVDYGGAATITYEGHEAGKCGIHSLTPGVPLRTSVVEVVAGANTVKTTFSSPTPEVTLEVEICSAIVKCASECTPPKEHVVAARPRHGSDTGGYISGPAMRWAGRIVGNPSGPVSSSLAVTYCVVKKCRSKRIRIVKS

SEQ ID NO: 23 - DNA, 11137 bp, Artificial Sequence
  Infectious haematopoietic necrosis virus (IHNV) complete genome
gtataaaaaaagtaacttgactaagctcagaaggacacaaggacagaaaaaagaaaaaggtggcacttcagttgtaacctgataacgtgcttcagacactataaaccgagacagaacaagcagaactattttcactgaaaacaacacctgagagacagaaacggatcacgaacgatgacaagcgcactcagagagacgttcactggactcagagacatcaaggggggagtcctcgaagatgcagagacggagtatcgtcccggtacgataaccctccctctatttttctccaagacagactttgacctagagatgatcaagcgggcggtgagtcaagtcggaggagagggaacgagaagggcattgggcctcctgtgcgcgttcgtcattgcagagacggtcccatcggaggcaggtacggtcgccgaacttctggaagccctgggctttgtgctggagtctttggacactggggcaccaccagacgctaccttcgcagaccccaacaacaagcttgcagaaacgatcgtaaaggaaaatgtccttgaggttgtgaccggcctcctcttcacctgcgccctactgacaaagtatgatgtggacaagatggccacatactgccaaaacaagctcgagcgtcttgcaaccagccaagggattggcgagttggttaacttcaacgccaacaggggagtcctggccaggatcgggtcggtgcttagacccggacagaagctcaccaaggctatctatgggatcattctcatcaacctgtccgacccagccatcgctgccagagccaaggcactgtgcgccatgagactgagcgggacaggaatgacaatggtgggactgttcaaccaagccgcaaagaacctgggcgcccttccagccgaccttctagaggatctgtgcatgaagtcagtggtggagtccgccagacgcattgtcagactgatgaggatcgtagcagaggccccaggggtagcagcaaagtacggtatcatgatgagcaggatgctcggggaggggtacttcaaggcctacgggatcaacgagaacgccaggatcacctgcattctcatgaacatcaacgataggtatgacgatgggacctcgagaggactgacagggataaaggtatccgaccctttcaggaagctggcgagagagatcgctcgtctccttgtcctcaagtacgatggcgatggctcaaccggagagggggcgtccgagctgatccgccgggcggagatggcatctcggggaccagacatgggtgaggaagaggaggaggacgaggaggacgactactccagtgagccaggagactccgattcattccgctgaacacaaacagcccccccctcctttttcctctcccgcccctcgacccatccagatgtttactccaccctgactcttagatagaaaaaaatggcactatagtgcttttcaactcaaaccacaacaatgtcagatggagaaggagaacagttcttcaacctagaaggcgaagacatactgaggctagaatcccgcctgaaaaacccacggaatgacgggcaaatcggcaagaaccccaggcaacggaaggaggaccaggcgcctcaagaggaaccaaagaaaaccaccaggagacccgacaagaacaagggtctatctcaactggagcaactcatcctaaagtacgttgaggaggagagctgtcaggatgccctgaaggacttcggaggtctaattgccaacatcagacaggcccaccaggccgaactgacatctcacctagaaaaggttgctacggaacaccgagccaatcttcaggctcttacaaagtcccagcaagagcacgagaaagtctcgaaggagattttgtctgcggtaattgctattcggtccaacctcaacgagaaccacagtcccaggcccaagccactcgacccggatcaggtgaaggccgctcgtgcccttggattcggaattgggtatcgaacggcccttaatgtcttcgaccgaataaagggagtcaccccagacaacgcaggatcccaagaggtgaagaacatggccattcgggcggcggaggaggatgaatacgagggaagtcccactttcttcaggaaggtgatagacgccgtaaagaagcgcatgaagcaaggtcaatagacatcctcctccaggatcccggctaccaagacagaaaaaaatggcacgcaagtgtatcgttccaaacgaaatccaagagtcagttcaaacgagagcatgtctattttcaagagagcaaagagaacagttctgatccctcctcctcacctcctcagcggagacgaggagagggttacaatactcagtgcagagggggagatcaaggtgactggaagaagaccaaccacacttgaggagaagatctattactctatgaatctggcggctgccatcgtagggggagatctacacccctcattcaaatccatgacctatctgttccagaaagagatggagttcggaagcacccaagaaaaggtcaacttcggctctcggaaaccagcacctcagaccacctaccaggtaatgaaggcgagggaaatctaccttcagacccagcctctcgagaagaagatcccaatgcagacctactcagtcagcacagagggggctaccatcaccttcacaggacggttcctgttttcatccagccatgtaggttgcgacgacaacaggaccaaactggcggggcttgacgggttcacaacctccaacagctaccagagggtcaaagactactatgcccaggagacagccctggccctgaccttcgcggctcccgaaaagcgggggaaggaaaaataggagtcgaccatgccgtctctcactcacccatccatcggccgacccaaccctcctccatccccagagtccctccactcctcactccgtccaagacagaaaaaaatggcacttttgtgctttgagacagaacgcaactcgcagagacccaccaaaacaatggacaccacgatcaccactccgctcattctcattctgatcacctgcggagcaaacagccaaaccgtcaaacccgacaccgcaagcgaatcagaccaacccacctggtcaaacccgctcttcacctatcccgagggatgcactctggacaagctctccaaggtcaatgcctctcagctgagatgcccaaggatcttcgacgatgagaacagggggctaattgcctatcccacatccatccggtccctgtcagtcggaaacgacctcggggatattcacacccaagggaaccacatccacaaagtcctgtaccgcaccatctgctcaacagggttcttcgggggtcagacgatagagaaggcgcttgtagaaatgaaactctctacgaaagaagcaggggcgtatgacaccacaaccgcagccgctctgtacttcccagctccccgatgccaatggtacaccgacaacgtacaaaacgatctcatcttctactacacaacccaaaagagtgttctgagagatccctacaccagagactttctggactcagattttattggaggaaaatgcaccaaatcaccctgccagactcattggtccaacgtcgtttggatgggtgatgcagggataccagcctgtgattccagccaagagataaaaggtcacctctttgttgataaaatctccaatcgagtcgtgaaggcaacgagctacggacaccacccctggggactgcatcaggcctgtatgattgaattctgtgggaaacagtggatacggacagatctcggtgacctaatatctgtcgaatacaattctggagcagaaatcctctcgttcccgaagtgtgaggacaagactgtggggatgaggggaaacttggatgactttgcctatctagacgatcttgtgaaggcctctgagagcagagaggaatgtcttgaggcgcacgccgagataatatcaacaaacagtgtgactccatacctcctatccaagttccgatctccacatcccggaataaatgacgtctacgctatgcacaaaggctccatctatcacgggatgtgcatgacggtcgctgtggacgaggtatccaaggacaggacaacttacagggcccatcgcgctaccagtttcacgaaatgggaacgaccctttggggatgagtgggagggctttcacggattgcacggaaacaacaccaccattattccagacctggagaaatacgtcgcccagtacaagacgagcatgatggaaccgatgagcatcaaatcagtaccccatccaagcatcctggccctctacaatgagacagacgtatcagggatctccatcaggaaattggactcattcgaccttcaatcactccactggagtttctggcccacaatctccgcactgggtgggattccctttgctctcctccttgctgttgccgcgtgctgctgctggtcagggagacctcccactccctccgcgtcgcagagtatccccatgtatcacctggcaaaccggtcctaaaggacctcaatcctcacttcctccccaccagacagaaaaaaacggcacatctgtgctgcaaaaagagacaatggaccaccgcgacataaacacgaacatggaggcactcagagaagttctgcgatacaagaacaaggtggccggacacggcttcctctttgacgacggagacctggtatggcgtgaagaggacgacgcaacgtggaggcggctttgcgatgtcgtcaacgcactgatctcctccaagaggatgcagcgagtattgtacatggacctcagcatcaccaagggcgaggggcatctactttttgtggatctccaggggaccaagaaccgcctgcacaaagaaccccgattcaggagacatctgatcctgattgaagactttcttgcttatcccagatagaaaaaaatggcacttttgtgcaaaaaaactcaagcgcgactcacagagaacacaaccagcaacgacagcaacccatcacagaaaaatggacttcttcgatcttgacatagaaataaaacaagagagacttccagcggaatgctccctcaactcacccctaaattactcactgtctgcacaactgacagatcgcatgacccctagaactgagaacgtcaggaggcaaagagaaaggctcaggtcccacatgcgagaacacttccgggtcaaggatctatccaccctcgacaatgactccaccaggctccatgcacgactcacagaggatctcatcaccattcaaagccccgagatcgattcctcagtccttgaaaattggaccccacttaagtcctactatgcatctctagactacacactcccggaaaagacagccttcaagtgggaacacgcagccccctactggaatctatttgcacaactgagggcaattctcctccagagccaaaagatcaagaaacaagagacgggcacgagggaactctactcttgtggccctcttcagatagagttcgtggagggggtagtcttgtacttcacagatcgaggatcgaaagaggaattcaccaagtcaggagagctcccaagtgtaacaccgtatgcggacttccttgcctggatcaagatcatctcccaacgggcccaggccgtgttgatggcagtcatcctcagagtgaccgacaagggactatctccactaccagaatcactcctggccatctatcaaaacgtggacgacatcctgaaacgagcgggccagcctgcgatagacctcctgaaactctgggagccattggtgattacaaaattgggagaacttctcggagacagattcggactagaagaagatttccgaaacacaatacgaggtgaggcaacaaaattggcaaagaacctattcatttcacgcgggctcaatcgcctcatggacatcctggatcagcaaactgacgctcaaccgctctttcaatttttcggactattcaaacattttgcgtacccacgtgtcttctcaagagacaccatccaggcaatccaggaggtaagtgacaggcctagctcaatctcagctgtcgactttctccatgatcagtgcgagattcgcaaggaattctacgtcagatacataaacgcataccacagggcacctggccttgacctctccgccctgagtccctcctcatttctaagagacagtctcgagcgtggaaaaatcccaaacgaaagaagccctctctattcaaacaaagagtggtacttcgtcaaattcacaaaaagcatcgagtggccagttagcgacactctgtcgacattcctatcggacaaagcgatcactcgtgaccgacccgcctggattgaggacgggcactcgggtagagacatgtccgagaagcggctgctactcaaatttatcaaggagaacttctcgagtgttgcagacattgtcggtgcggcggaagcgatttacaacaaccaagaggaccgactgatcgcactgaaagtgaaagagatggaactaaagatcaagggcaggggattcgggctgatgaccttcatgcctcgtctcctccaagtcctccgtgagagcatcgccaaaaaaacacaaaaactattccctgagatcaccatgacctcctcagatctggacatgaaaaaaagaaagttcatgctctcaaaaaagagcgacgacaggcgaggcttcatacacgtgaataaaagtctggacatcaacaaattttgcaccagccagagacaattcaactccaatgcggttttctcaagtctagatgaactcatggggacctttcctttgttttcccgagttcacgaaatttttgaaaaaacctggatcgtagacgggtcatcctcggatccccccaacttggcacacttcactagaattctcgacgaatgcacggcacttggtctagacacccctcacatctgggcggacggggtcttctcggggttgaagggagggatcgaagggctctgtcaatacgtttggactatctgtcttttacttagagtagaacgcgtcatgcaaaaaacagcgctcacccactacattctggcacagggcgacaatgtgattatcacaattatcgtaccagtagaaatacatcgggatggcaccattccagaacaagagtcccgtcgtatcttggccttaagtcgagacatcgatctctcactggagagtgaactagaaaaaagtggtctcaccctaaagatcgaagagactctaacaagtgagaacatctccatctacgggaaggatcttcactgtccgcaacaccttaccctggcaatcaagaaggccgcctccgccgcgatcatatcaagtgagcagtaccaagatgtcccaacatttctatctggtctcggaacgagtctggaggccctctcagagtgtgtcaacaacaaagtcggggtgcatctctttggagtgatcatgggcgttgcgggatggagggacctggcgactcatcagacttggcgaggatggagatacccctatcacaagaaggcgataactgggcggattcgggcatccgagatgaagctcagcaaaggcgaacccaccgagctgtccatctctgtcctctccaagcgacgtagagagaccgaaacactcattgaactactatccaacagcctgctcgggtcagccctaggaatgcttgcgttccctactcctctagacctggaaaaacggggtgtgggagactacatcacccatcggctcaccatagcacgcaaggcccttctgagtggacaccttgatccccgtatcgggagaaaagttgaatcagcatgcaacatccccctctcgtccagaaccgacctaagcaaactgtttgactctccattttcattgaacatcgcaaccgaagaggacgcgacggcagtcatcaaacgacaggcaacaaaaatcctccgactgcaagagataaaaaacgagaagctgaaggcccagattgacaacatggacaagggcatagcgacccttgatgcagcacttgcaggagccaccaacatcaaccccagactgaattacatgatcaggagcatcacggacgaaaaggagtccgagatgtttgtcactaaattcgcatcagcaagaaccatgaggaccctggcaatgaatcactcctcagaacttcccattgtcatcctcctggagatgaaaagccaacaaaaagagacatacacaatctggagaaccaagagaccacctgtgaccatgtggaaatgctccacagtgttggcgaaggagctccgagacacctcctggggcaaaaacataatcggaggcacatccccttctccaatcgaggcaatggagacaatccaaattgaccccaccgaatgggaagaccgacggtcccaggacgccatgtccatcaactactatctgtcacgggctggcatggacgaacagacagcaaagctcactcgagggttcctagtcccttactacgggacccaaacaaaaccccttgtggccaaggcctacctagaactcaagggcaacccccgaaccaacaaggctctccttctcctgagtgttcgagagtctctcgtaaaaacaggcagcaatctcgacaagttgatcataaagctctgctcccatgcactagacatcgatgtagcctcactccccgcccttcgagcacaggaagaggcggcagctggggagggtctccgaggaggaatcaaagaatccatgtctccagtaggtcccgataacttctacacaaacatcacgcacaaggtcttcaacaggaagtgggcaacaccataccacgtcaacattgcagacttcatcatccaaggcctgatagagaccagaaggcacctccttgtaaatgagaggatgaatgggctactcccggtcagctcagtcaaatgcacctcctgcttcaggaagaaagagagggagttctttgatattcccgaggaattcacctggaaaaatgagtcaaaaacctcagatcccgcctacacctacttcaccacctggtgtgacctccccagggtctccaacctgcccgagatggatcaacggtcagccactcgtctcttgggaagagggctcgcattaaacagatcctcctccggtgaaatcatcaccaaattctactcaatgcccatggaatcacaacggctccttcaccccgtggaactgctcctgggatatggtgaaggggttatatttggatatttgaggagtcagcacataaatcacggagccctctttcacattggcgacgagtcactggcaaagaaactcagacgttatgtcctagacacaaagacacaacatgccaagcaaattggctacctcttccaggatgaggactcattacatgagctcctgggccaaggactttgtccatatatacccaggtcaatccctcttaccatcactgaactaacaaacgcctgcgcgatcacaaccatcagggcaacggaggtaatcctaagcacaaaagccaggatccaccatatgcccgtccaggcaatcgacgaatcagatgtcgacaccagcagactagctgcgaacaccatgcagaccatactcggagaccccagacccatgaacctcgtgcatcttgactgcgatctcacccacaacatggtcgcatgggagtcagaagtggaactcgacatcttgaaatcagaaaacttccacattgacggactcctggtagaactcactgcccgcgagctccccattggagacaccccctggaagcaaagagactggacctgttccaacgatcccaagatcatcgccaagggcatcaagacaaaatccctgttcattcatcagggcgtgactggggcaatcaacttgattcccgatcttctggtcgtgatcgggggaggtctcggagggtgcgccgttccctaccttcaggaatggccagacacaccaatcatctttgcaaccctattcgacgaacgggagaggatctccgaggatggagacttgattgttcccccagagctcctggtgaggggcatggcacctaggatgatcgagagggagatcttagaagcagagctgtgtgacatcaccaatgaggggaatcgtcgcctgctaattcgactggtcacgaaaaacaagggaggaggcaaggtggtgctcatagacgagatcgagaatcgaggggccccagaatcccttctacagagctcgctccaagacctattcgagaaactggacaaagtctgcaaactcacctctgtgcacacagtcagagagtcaacagtggaacaattctcccagagggtaaattctatcaaacgcagcaggaaggcagtaacactgcattggaaccgctacaacagaagagaccaatttgaggccctggtcatcgtgaagggcgaagaggccaagtccgactaccggatctccacgatcacctcggcaaaggcatttcgcaagatcgacgaacagctcgagatagacgggagactctcgtcgacacagtggtccctccccgcgcttccttctcgagaaaaaaacattctttttgggtacgtgtcatccgtcttccttaagatgaaccttgcactctcagccaacgacatggaccgagagcgactgatcgagaccattgagggaaccgccccagggttgatctcctggaaggaaaaactcgaacacagggaccatgcacatcgctccgacattgaagagaaaggaataactcaggacaagatcttcaacctcatctgcctctcctgggtcctcaagggtctgagatacggagtatgggacaccgacgctcaatccattgtagctcaaacggtgtacatcactcgagggccaaagctctgtccattgggggagaagcccaagcgcatcttcgcatcattcaaactacaaagcggaaagcgagttgaagatgccaagggattcctgagcgcactgcttcaccttgagggcttctttccactaggcgaacaataggtacactccagcagcacccagtgccagggaccaaactcccagatacaaaaaaatggctcccgactcccatggtaagggaaccagtcattgttttctgttattaaaaaccttttttaactgccttttgagaacccttctgttactttttttat

SEQ ID NO: 24 - protein, 391 aa, Artificial Sequence
  Infectious hematopoietic necrosis virus (strain Oregon69)
MTSALRETFTGLRDIKGGVLEDAETEYRPGTITLPLFFSKTDFDLEMIKRAVSQVGGEGTRRALGLLCAFVIAETVPSEAGTVAELLEALGFVLESLDTGAPPDATFADPNNKLAETIVKENVLEVVTGLLFTCALLTKYDVDKMATYCQNKLERLATSQGIGELVNFNANRGVLARIGSVLRPGQKLTKAIYGIILINLSDPAIAARAKALCAMRLSGTGMTMVGLFNQAAKNLGALPADLLEDLCMKSVVESARRIVRLMRIVAEAPGVAAKYGIMMSRMLGEGYFKAYGINENARITCILMNINDRYDDGTSRGLTGIKVSDPFRKLAREIARLLVLKYDGDGSTGEGASELIRRAEMASRGPDMGEEEEEDEEDDYSSEPGDSDSFR

SEQ ID NO: 25 - protein, 111 aa, Artificial Sequence
  Infectious hematopoietic necrosis virus (strain Oregon69)
MDHRDINTNMEALREVLRYKNKVAGHGFLFDDGDLVWREEDDATWRRLCDVVNALISSKRMQRVLYMDLSITKGEGHLLFVDLQGTKNRLHKEPRFRRHLILIEDFLAYPR

SEQ ID NO: 26 - protein, 230 aa, Artificial Sequence
  Infectious hematopoietic necrosis virus (strain Oregon69)
MSDGEGEQFFNLEGEDILRLESRLKNPRNDGQIGKNPRQRKEDQAPQEEPKKTTRRPDKNKGLSQLEQLILKYVEEESCQDALKDFGGLIANIRQAHQAELTSHLEKVATEHRANLQALTKSQQEHEKVSKEILSAVIAIRSNLNENHSPRPKPLDPDQVKAARALGFGIGYRTALNVFDRIKGVTPDNAGSQEVKNMAIRAAEEDEYEGSPTFFRKVIDAVKKRMKQGQ

SEQ ID NO: 27 - protein, 195 aa, Artificial Sequence
  Infectious hematopoietic necrosis virus (strain Oregon69)
MSIFKRAKRTVLIPPPHLLSGDEERVTILSAEGEIKVTGRRPTTLEEKIYYSMNLAAAIVGGDLHPSFKSMTYLFQKEMEFGSTQEKVNFGSRKPAPQTTYQVMKAREIYLQTQPLEKKIPMQTYSVSTEGATITFTGRFLFSSSHVGCDDNRTKLAGLDGFTTSNSYQRVKDYYAQETALALTFAAPEKRGKEK

SEQ ID NO: 28 - protein, 508 aa, Artificial Sequence
  Infectious hematopoietic necrosis virus (strain Oregon69)
MDTTITTPLILILITCGANSQTVKPDTASESDQPTWSNPLFTYPEGCTLDKLSKVNASQLRCPRIFDDENRGLIAYPTSIRSLSVGNDLGDIHTQGNHIHKVLYRTICSTGFFGGQTIEKALVEMKLSTKEAGAYDTTTAAALYFPAPRCQWYTDNVQNDLIFYYTTQKSVLRDPYTRDFLDSDFIGGKCTKSPCQTHWSNVVWMGDAGIPACDSSQEIKGHLFVDKISNRVVKATSYGHHPWGLHQACMIEFCGKQWIRTDLGDLISVEYNSGAEILSFPKCEDKTVGMRGNLDDFAYLDDLVKASESREECLEAHAEIISTNSVTPYLLSKFRSPHPGINDVYAMHKGSIYHGMCMTVAVDEVSKDRTTYRAHRATSFTKWERPFGDEWEGFHGLHGNNTTIIPDLEKYVAQYKTSMMEPMSIKSVPHPSILALYNETDVSGISIRKLDSFDLQSLHWSFWPTISALGGIPFALLLAVAACCCWSGRPPTPSASQSIPMYHLANRS

SEQ ID NO: 29 - protein, 230 aa, Artificial Sequence; identical to SEQ ID NO: 26
  Infectious hematopoietic necrosis virus (strain Oregon69)

SEQ ID NO: 30 - protein, 1986 aa, Artificial Sequence
  Infectious hematopoietic necrosis virus (strain Oregon69)
MDFFDLDIEIKQERLPAECSLNSPLNYSLSAQLTDRMTPRTENVRRQRERLRSHMREHFRVKDLSTLDNDSTRLHARLTEDLITIQSPEIDSSVLENWTPLKSYYASLDYTLPEKTAFKWEHAAPYWNLFAQLRAILLQSQKIKKQETGTRELYSCGPLQIEFVEGVVLYFTDRGSKEEFTKSGELPSVTPYADFLAWIKIISQRAQAVLMAVILRVTDKGLSPLPESLLAIYQNVDDILKRAGQPAIDLLKLWEPLVITKLGELLGDRFGLEEDFRNTIRGEATKLAKNLFISRGLNRLMDILDQQTDAQPLFQFFGLFKHFAYPRVFSRDTIQAIQEVSDRPSSISAVDFLHDQCEIRKEFYVRYINAYHRAPGLDLSALSPSSFLRDSLERGKIPNERSPLYSNKEWYFVKFTKSIEWPVSDTLSTFLSDKAITRDRPAWIEDGHSGRDMSEKRLLLKFIKENFSSVADIVGAAEAIYNNQEDRLIALKVKEMELKIKGRGFGLMTFMPRLLQVLRESIAKKTQKLFPEITMTSSDLDMKKRKFMLSKKSDDRRGFIHVNKSLDINKFCTSQRQFNSNAVFSSLDELMGTFPLFSRVHEIFEKTWIVDGSSSDPPNLAHFTRILDECTALGLDTPHIWADGVFSGLKGGIEGLCQYVWTICLLLRVERVMQKTALTHYILAQGDNVIITIIVPVEIHRDGTIPEQESRRILALSRDIDLSLESELEKSGLTLKIEETLTSENISIYGKDLHCPQHLTLAIKKAASAAIISSEQYQDVPTFLSGLGTSLEALSECVNNKVGVHLFGVIMGVAGWRDLATHQTWRGWRYPYHKKAITGRIRASEMKLSKGEPTELSISVLSKRRRETETLIELLSNSLLGSALGMLAFPTPLDLEKRGVGDYITHRLTIARKALLSGHLDPRIGRKVESACNIPLSSRTDLSKLFDSPFSLNIATEEDATAVIKRQATKILRLQEIKNEKLKAQIDNMDKGIATLDAALAGATNINPRLNYMIRSITDEKESEMFVTKFASARTMRTLAMNHSSELPIVILLEMKSQQKETYTIWRTKRPPVTMWKCSTVLAKELRDTSWGKNIIGGTSPSPIEAMETIQIDPTEWEDRRSQDAMSINYYLSRAGMDEQTAKLTRGFLVPYYGTQTKPLVAKAYLELKGNPRTNKALLLLSVRESLVKTGSNLDKLIIKLCSHALDIDVASLPALRAQEEAAAGEGLRGGIKESMSPVGPDNFYTNITHKVFNRKWATPYHVNIADFIIQGLIETRRHLLVNERMNGLLPVSSVKCTSCFRKKEREFFDIPEEFTWKNESKTSDPAYTYFTTWCDLPRVSNLPEMDQRSATRLLGRGLALNRSSSGEIITKFYSMPMESQRLLHPVELLLGYGEGVIFGYLRSQHINHGALFHIGDESLAKKLRRYVLDTKTQHAKQIGYLFQDEDSLHELLGQGLCPYIPRSIPLTITELTNACAITTIRATEVILSTKARIHHMPVQAIDESDVDTSRLAANTMQTILGDPRPMNLVHLDCDLTHNMVAWESEVELDILKSENFHIDGLLVELTARELPIGDTPWKQRDWTCSNDPKIIAKGIKTKSLFIHQGVTGAINLIPDLLVVIGGGLGGCAVPYLQEWPDTPIIFATLFDERERISEDGDLIVPPELLVRGMAPRMIEREILEAELCDITNEGNRRLLIRLVTKNKGGGKVVLIDEIENRGAPESLLQSSLQDLFEKLDKVCKLTSVHTVRESTVEQFSQRVNSIKRSRKAVTLHWNRYNRRDQFEALVIVKGEEAKSDYRISTITSAKAFRKIDEQLEIDGRLSSTQWSLPALPSREKNILFGYVSSVFLKMNLALSANDMDRERLIETIEGTAPGLISWKEKLEHRDHAHRSDIEEKGITQDKIFNLICLSWVLKGLRYGVWDTDAQSIVAQTVYITRGPKLCPLGEKPKRIFASFKLQSGKRVEDAKGFLSALLHLEGFFPLGEQ

SEQ ID NO: 31 - DNA, 11065 bp, Artificial Sequence
  Viral hemorrhagic septicemia virus strain FA281107, complete genome
aataacggcacataagtggcaaaaagttttcaagtttgaaattccaagaatttggaaagtgaaagttgaacacagagtcatatctcgtaatctttgaacaaaagaactcagttgaagaatggacggaggacttcgtgcagcgttctcaggtttgaatgacgtcaggatcgaccccaccggtggagagggacgggtacttgtacctggtgaggtggagctcattgtgtatgtgggtgggtttagtgcagatgatgggaaggtgatcgtagatgcactttctgcacttgggggaccccagactgtgcaggcattgtccgttcttctctcctatgtacttcaagggaacaaacaggaggacctggaggcaaagtgcaaggtgctcacagacatgggcttcagggtgacacagtcaaccagggcgacgggcatcgatgcaggaatcctgatgcctatgagagagctggccctggccgtcaataacgacagcctcatggacatcgttaaggggaccctgatgacatgttcccttctgaccaagtactcggtggacaagatgatcaagtacatcaccaagaagcttggggacctggcagacactcagggaattggggaactgcagcacttcaccgccgacaaggcagccatcaggaaactcgcagggtgtgtgcgtcccgggcagaagatcaccaaggccctctatgcattcatcctgaccgagatcgcagatcccaccacccaatcaagggcccgagccatgggggcattgaggctcaacgggacaggaatgaccatgatcgggttgttcacccaggccgccaacaacctgggcattgccccggcgaagctgctggaggacctctgcatggagtctctggttgagtcagccagacggatcatccagctgatgagacaggtgtcagaggcaaagtccatccaagagcgctacgccatcatgatgagtcggatgctgggggaatcctactacaagtcgtacggactaaatgacaactccaagatctcctacattctgtcgcagatcagtgggaagtacgcagtggactccctggaaggcctggaggggatcaaggtgacagagaagttccgcgagttcgctgaacttgttgccgaggtcctggtggacaagtacgagaagattggggaggacagtactgaggtttcagatgtcatcagggaggcggctagacagcacgcgcgcaggacatccgccaagccagagcccaaggcccgcaacttcaggagccccaccggaagagggaaggagcaggatacagaggagtctgacgatgaggactaccccgaagactccgattaaaaagcactctcgtctcatgaccaacatagatagaaaaaaatggcacgattataagaatttctctttgatcaaagcaaggcaaacaccgaaatcacacaatggctgatattgagatgagtgaatccttggtcctgtcccatgggtccctagctgacctggataggaaactagacaacgcccccaaggacactaggtcagctcttttttcatctacctcagggttcaccaaacaaaaatcaagtcccaagaagaagcccaatcctacaacgctggaagaactcattggacacttcgtctctgaggatctccagctagacgccacaaaggccttcggacagctcctaagacgtatcaagatgtcccatcaggaagaacttactcaacatctggagaaggtcaacggagagaaccgggccaagatgggagccctcctggagtctcagaaagaaaatgggaagaagaccgacaacatactctcaatcctcatctccatgcgaggagaaggggcggaaaatgcatccaaaaagcccaaggtcctagatggggatcaggtccggaacgagagggcacttggattcaacaggggactgactacggctgccatcgccatgaagaagttcaagttagaagatcctctcgcactctgcaaaggctcagtcaaacgagcagctctctccgccatggaaaaggaggaatacgacggggagcgagagacctactccacagtctcaaaagcgattaaggcggagctggacaagctagagtagggacaacacaatgcatcgtacagatagaaaaaaacggcacgactgttcgagcaaacttactcccaaaaagagacactccagcagccagcaaaccccagacaatccacaactcaagacatgactctgttcaaaagaaagcgcaccatcctggttccccctcctcacatcacgtccaacgacgaggaccgtgtctcaaccatcctgacggaaggcactctaaccatcacgggtcctcctccgggaaaccaggtcgacaagatctgcatggctatgaagctggctcgagccattctctgcgaggatcagcacccagcattcaatcccctggtttatctattccagagtgctatgatctttggagagaccagtgagaagattgacttcgggactcgtagcaagaccctgatcacaagcttcaagatagctgaggcaaaggccatctacctggaatcaagtccggtgagatctcgcatagaagccaagaagtataccactcccatcagacatgggagtgtgacttactatggcccgttcgtgtttgcggatgaccacgttggagggaaaggccaccgcgagaaactgggggcactatgtgggtttctccagtcgggaccttacgggcaggcgaaggactactacaatcgtgctgtcgaggaagaaatgggcattctcccgagggacccaaagcgcagatctggagcctcttctgctcagccccggtaggtccaaatgacaaactcaggggtgataccatactcccattagatagaaaaaaatggcacatttgtgtacacaacaagctatacctacaatggaatggaatacttttttcttggtgatcttgatcattctcatcaagagcaccacatcacagatcacccaaagacctccgatcgagaacatctcgacgtaccatgcagattgggacactccgttgtacactcacccctccaactgcagagaagactcctttgtcccgattcgaccagctcaactcaggtgtcctcatgaatttgaagacatcaacaagggactggtctccgttccaactcagatcattcatctcccgctatcagtcaccagcgtctccgcagttgcaagtggccactacctgcacagagtgacctatcgagtcacctgctcaaccagcttctttgggggacaaaccatcgaaaagaccatcctggaagcaaaattgtctcgtcaggaggccgcaaatgaggcaagcaaagaccatgagtacccgttctttcctgaaccctcctgcatctggatgaaaaataatgtccataaggatataactcactattataagaccccaaaaacagtctctgtggatctctacagcaggaaatttcttaaccctgatttcatagagggggtttgtacgacctcgccctgtcagactcattggcaaggagtctattgggtcggtgccacacccacagcccattgtcccacttcagaaacactagaggggcacctgttcaccagaacccatgatcacagagtggtcaaggcaattgtggcgggccatcacccatggggactcacgatggcatgcacagtgacattctgtgggacagattggatcaagaccgacctgggagacctgatcaaggtggtaggacaggggggcgaaacaaaactgactccaaaaaaatgtgtcaatgctgacatccagatgaggggggcaacagacgacttctcatatctcaaccatctcatcacaaacatggctcaaaggaccgagtgtctagacgcccatagtgacatcactgcctccggtaaaatatcctcgtttctcctctcaaagtttcgtcccagccaccccggacccggcaaggcacattacctccttgatggtctaatcatgcgaggtgactgtgactatgaggcggtagtcagtatcaactacaatagcgctcagtacaagacggtgaacaacacatggaagtcatggaaacggatagacaacaatacagatgggtacgatgggatgatatttggggacaaactgatcatcccagacatcgagaaatatcagagtatctatgatagtgggatgctcgttcaaaggaacctggtggaagttcctcatctgagcattgtgtttgtctccaacacatctgatctctccaccaatcacatccacaccaacttaatcccctcggattggtcattcaactggagcctctggccgtcattatcagggatgggagttgtgggaggggccttccttctattggtactttgctgttgttgcaaagcgtctcctcctattccaaattacgggattcctatgcagcaattctccagaaatcagatggtctgaacacacctgcccgaatgaacatcattatcctcataggtagatagaaaaaaatggcacttctttgaaaaagaaacatgacaacccaactcgcgctcgacacagtcagcttttctccactcgtcctccgcgagatgatcacacacaggctcacattcgacccaagtaactacctcaactgcgacatcgatcggtcggacgtatccaccatgagtttcttcgaaacgactcttcccaggatcttggatgacttgagggcttgcccacggcttccctacctccatgtactcgacatgaggataagtctcctggagagaacccattacatgttcaaaaacgtcccctccagtcccgccacaaccggcagaccgactgatcctggactcatcattatttcatgtgcagatgttggggtcctaacaaatggctccgggttcacttcatgagcctcccccccgcccctcagatagaaaaaaatggcacttttgttgtttgtaatcctacgctcaacaaagaagtgaccctacagggggtaatacctgacacaatcaaattagatcaccttatagcaaccatggaaatgtttgaactagaccgggaagtacatcaggagcgacttccctccgaatgttcgctgaactctcccctaaacctttccctatctctccaactcttcgggcgcctaactccaaagacagaacacatcaggtaccaagctgggaggataaagagatggctcgtcaaacagtaccaactggtgcacctcagagagcttgagattgattcaacaagaatccaaggatatctgatcccccatcttttgaagacacagagtaatgagttaggatcgtcggtaataaaaaactgggggatggtctccaagtactacctgtctctagggtacacccttcccccaaaggacaagtttgactttcgagaagttgctccttactggaacttggcctcacagttgagagaggtcactttggagagccagaaggtggataccagagggaaagaaaaaagaaagttgtaccaggtggaagatgtagagttcgagtttaaagagggggtggtggtaattcgtgcgggcctggatggactcctgaatgagttccttgggggggaaaaacttggggcagtaacatatgtggaatacttggccttcttcaaaatcatcaatcaaagagcacaagcactcctgctcacggccatctgccaggcccttgaaccagacctagttcctccttgtagtggaatactttccgtctatgcagaggtcgactccgtactccgtcgggcgggacaatccgccattgacctgctcaaattgtgggaacctcttgtcctgacaaaactgggggacgtgctcggtgacagattcggcctcgaggacgacttcaaggacacaattaggggagaggcgaacaaattagctaagaaactccacgtcaccagatcttataaacggatgatgaagaccctagatcaggagactagggcacaggccctctttcaatattttggactgttcaaacacttcgcctacccccgcgtctattcgagagaaaccattgaggctattcaagaggtcagtgacaaaccgagtgactcgtccccactcaattatctaagtgaccaatgcaaaatcagggaggagttctacatccggtacacgaaggcttatcacagggcaccggccatgaacctgggtcaactaggacagggttcctacctacgccaggttttggaggcgggaaagatccccaacgccaagaatgccctctattcgctcctcgagtggttctttgttcgttttgagaagagcatcgaatggcccctcagtgacaccttatccaccttcctctcagacaaggcgatcacccaaaatagggacatctggtatgacgggggatcatcaggaagggacaccacagagaagagactactcctgaagtttatcaaggagaatgaagacagtgtggaaaaggtgatcctcaaggccgatgaaatctacgataaggaggcggatcagatcattgcactgaaagtcaaggagatggaactgaaaatcaaagggagaggatttggcttgatggctttcaagccaagactacttcaggtcctccgagagagtatcgccaagaagaccagcaagttattcccagaaataaccatgaccttttcagacctcgagttgaaaaagaaaaaatttcaactttctaggaagagcgatgacaggaggggatacatccatatcagcaaaagtcttgacataaacaagttctgcaccagtcaacgccagttcaactccctggcagtcttccagagcctagatgaacttctggggacagatcagctcttcacgagggtacacgagatcttcgaaaaaacatggatcgtggatggatccgccagtgaccccccagacctggtgacgttcaaggcgaggtatgaagaggcactggccctagggattgaggccccgcacgtgtgggcagacggagccttctctggactaatgggtggaattgagggtctctgtcagtacgtctggacaatctgtctcttactcagagtggagcgcgtcctcgctgtgacccaactgactcactttgtcatggcacaaggggacaatgtgatcatcaatctcatcatccctgttgaggtggaccgggtgggtggggttgtcgaaggggaacgtgccagaattcaacgcatcagcaaagacatcgactctgctctcgaacgagagctgctgaggagtggcttgactcttaagatagaggagaccctgaccagcgaaaacctctcaatctacggaaaagacctccattgcccacaacacctgacccttgccgtgaagaaggcaggatctgcctctatcatatcaagcgaacaatatcaagacgtgcccacctttctgtcaggcctcgggacgggaatggaaacaatctcagagtgcgtcaacaacaaagtgagtgctcatctgttcggggtcattctgggagcggctggatggaagagcctcgcacagagacaaacctggaaagggtgggaatacccctttcagaatgagacctcccggagacaggtccggtcacagggaatccttcttcagaagggggagtccacaatggtacacaaagagcccgaggccaacccggaaaaacggaccatagaactgctcctagtgagtagtcttttcggatcagctctggggatgctcccctttcccactcccattgaccttgagaagaggggcgtgggagactacgtcactcaccggctctcaatcgttaagatggccctggtctcaaaaaaactccccaataggatgatcgagatgattgtctccaccatgaaccttcccctttctcgagaacaggatctcactaagttattcgactctccattctccctcaatcttgccacagaggaagacgctgcatcagtgatcaagcgactcgcaaggggcacactaagggggcttgacatcaaaaacaaaaaactggcagatcacatagccaccatggatcagggaataacccaaattgacgaagcactggcaagtgcggacacaatcaaccccaggatcgcctaccaatttagaaacatcacagaccaaaaagagtcagaaatgttcgtcaccaagttcgccaccgcgaaaaccatgcggatggttgccctctcctcatctcaggatgtctcagttgtaggacttttgaacaaacgcagccaggcaaaagagatctacaccatttggcggacacagaggaagggggagacactctggacatgctcgacccagcaggcaaagaaactcagggaccgatcctggggaaaaaacataattggggtcacctctccttcccctctggaagccattctcttcaagctgattgatccaatttcctgggaggaagaaaaggaggcacaccacttcaccatccattactacctctcaaagcccagtctttcttcaaagacagcccacacgaccaggggtccacttgttccctacttcggaacccagaccaagccactgatcgcaaaagcctacatggagctcaagggaaatcccaggaccaacaaggctctgcagttgcttagcatgagggagaccatgatcaaggcagggtccaatctagacaaactcctcttgtccctgtgttctaacgccctagacatcgacgtcaactccctcccttcacttcaggcacaagaagaggccagtgctggagagggggtccgaggaggtatcaaagagtccatgtccccagttggaccggacaacctctacacacacatcacccacaaggtgttcgagcgtcagtggttgtctgagtttcatgtgaacatcgctgacttcatcatctggggaatcacgaagactcggcagcatctccaggtcaacactgacctgggggggagcctcccgatctgtgttccagcgtgtcaagagtgctacagagagaaagaacgtgtcttcctagacatcccaagggagactgagtgggtcaacacctccaccacttcagacaaggcccaaacctacttctccacctggtgtgatctcccaagagtgtccaccctcccctcactagaccagaaggacgcaacctgtctgatggggaggtccatggcaactcaaaaatctactccgggggaatccatcaccaagttctacagcacagccccagacacccatcgacttctccatcctgtgaccttggtcctgggctatgcagaaggaacaattttttcttacatgaaaagtcagcacaacattcatgggtccctttaccatccgaacatcgaagagatcgaaccagctcttgagaagtatgtgatcgacacgaagacatcccataccaagcacctgggatatctgtttcaagatgccgactccctccaggaactgctagagacaggaatgacaccatacattccccgatccattcctctcacgatcactgaactgacaagtgcatgctgcatgacacttgccaaagcaatctccattgtcctcagaaccggagtgaccattccattgatgccagaaaatggatacggagaaaacgatatccaagtggcaagactcaccgccaactccctttcccgactcatgcccagggggaggattcagctggtctacctggactgtgacctgacctcgcagatgaccgcgtgggtgccaaccagccagccgagcgtcctgggatctgtcaacttccacatcgagggggttaccatctccctcacagcaacagagatgagagtgggacaagaaagctgggaagagagaaagtggacatgttcaaacaacagacacatcatcgcaaagggggtcaagaccaaatctctgttcatacatcagagcgtccccgagatcatcacacacccgcccgacctcattgtggtgataggagggggcctagggggctgtgtagtgccctaccttcaaaagtggagaggtcccaaagtaatcttctgcactctctttgatgaacgagagcggatctcggaggacggagacctgatcattcccccggagctcctagttcgaggactcgctgggagaatgattgagaaagaactcctggaggctgagatgtgtgacatcacggtcaaggggaatagggacctcctcatcaaagtagtgcagaaatgggttcagccgaacgaacatgtccttctcattgatgagattgaaaataggggggatcaagagtcagtactccaatcttccatcagtgagcttctgacaagaatggacagcgtgtgcaacctcacttctgttcacaccatccgagagaccggacctcgacaattcgcccagagggtgaacacgattaggagaggcagaaggacagcaacactccactggaaccaatacaaccgaagagaccaggtggaggccctcctacttgttgagagccacaccaggaagaccgaactgcacgtcacatcatccgccgtacaggcggcattcagaaagatagacgagaagctcgagtcggagagtcgactagagcacagcaaatggtcactccctgaactccctccaagggagaaagacatcctacttggatatgtggcatctgtctttttaaagctagggctggttgtgacagacagacacatgtcggctgccgcgctcatcactctcctcgaagaagctgggccaaaaatgatatcctgggataagaagatggaacatcgaacctgggcctcctcggatgccatcactgagaaaggaatcacccaggaccagatcttttcccttctctgctttgcatgggcccttaggggactaaaatcaggggactgggaacacaacgcagacgccatcattctccaggatgtccacatcgacacaggtccgaggctctgtcagatgggcgagtctcccaagaggacatttgcatccttcaggctgcacaacaccaaaaaggcggaggatctcaaggggtacttaggtgctctgctccacctggagtccttctttccatttggagaacagtgacgatcgccccgctcatcaattagattgaaaaaaactatgacctgggttcaatgaccacgtgaaccctcgtgggcaccatgtcccagacctcccgcctcggattt

SEQ ID NO: 32 - protein, 404 aa, Artificial Sequence
  N protein [Viral hemorrhagic septicemia virus]
MDGGLRAAFSGLNDVRIDPTGGEGRVLVPGEVELIVYVGGFSADDGKVIVDALSALGGPQTVQALSVLLSYVLQGNKQEDLEAKCKVLTDMGFRVTQSTRATGIDAGILMPMRELALAVNNDSLMDIVKGTLMTCSLLTKYSVDKMIKYITKKLGDLADTQGIGELQHFTADKAAIRKLAGCVRPGQKITKALYAFILTEIADPTTQSRARAMGALRLNGTGMTMIGLFTQAANNLGIAPAKLLEDLCMESLVESARRIIQLMRQVSEAKSIQERYAIMMSRMLGESYYKSYGLNDNSKISYILSQISGKYAVDSLEGLEGIKVTEKFREFAELVAEVLVDKYEKIGEDSTEVSDVIREAARQHARRTSAKPEPKARNFRSPTGRGKEQDTEESDDEDYPEDSD

SEQ ID NO: 33 - protein, 222 aa, Artificial Sequence
  P protein [Viral hemorrhagic septicemia virus]
MADIEMSESLVLSHGSLADLDRKLDNAPKDTRSALFSSTSGFTKQKSSPKKKPNPTTLEELIGHFVSEDLQLDATKAFGQLLRRIKMSHQEELTQHLEKVNGENRAKMGALLESQKENGKKTDNILSILISMRGEGAENASKKPKVLDGDQVRNERALGFNRGLTTAAIAMKKFKLEDPLALCKGSVKRAALSAMEKEEYDGERETYSTVSKAIKAELDKLE

SEQ ID NO: 34 - protein, 201 aa, Artificial Sequence
  M protein [Viral hemorrhagic septicemia virus]
MTLFKRKRTILVPPPHITSNDEDRVSTILTEGTLTITGPPPGNQVDKICMAMKLARAILCEDQHPAFNPLVYLFQSAMIFGETSEKIDFGTRSKTLITSFKIAEAKAIYLESSPVRSRIEAKKYTTPIRHGSVTYYGPFVFADDHVGGKGHREKLGALCGFLQSGPYGQAKDYYNRAVEEEMGILPRDPKRRSGASSAQPR

SEQ ID NO: 35 - protein, 507 aa, Artificial Sequence
  G protein [Viral hemorrhagic septicemia virus]
MEWNTFFLVILIILIKSTTSQITQRPPIENISTYHADWDTPLYTHPSNCREDSFVPIRPAQLRCPHEFEDINKGLVSVPTQIIHLPLSVTSVSAVASGHYLHRVTYRVTCSTSFFGGQTIEKTILEAKLSRQEAANEASKDHEYPFFPEPSCIWMKNNVHKDITHYYKTPKTVSVDLYSRKFLNPDFIEGVCTTSPCQTHWQGVYWVGATPTAHCPTSETLEGHLFTRTHDHRVVKAIVAGHHPWGLTMACTVTFCGTDWIKTDLGDLIKVVGQGGETKLTPKKCVNADIQMRGATDDFSYLNHLITNMAQRTECLDAHSDITASGKISSFLLSKFRPSHPGPGKAHYLLDGLIMRGDCDYEAVVSINYNSAQYKTVNNTWKSWKRIDNNTDGYDGMIFGDKLIIPDIEKYQSIYDSGMLVQRNLVEVPHLSIVFVSNTSDLSTNHIHTNLIPSDWSFNWSLWPSLSGMGVVGGAFLLLVLCCCCKASPPIPNYGIPMQQFSRNQMV

SEQ ID NO: 36 - protein, 122 aa, Artificial Sequence
  NV protein [Viral hemorrhagic septicemia virus]
MTTQLALDTVSFSPLVLREMITHRLTFDPSNYLNCDIDRSDVSTMSFFETTLPRILDDLRACPRLPYLHVLDMRISLLERTHYMFKNVPSSPATTGRPTDPGLIIISCADVGVLTNGSGFTS

SEQ ID NO: 37 - protein, 1984 aa, Artificial Sequence
  L protein [Viral hemorrhagic septicemia virus]
MEMFELDREVHQERLPSECSLNSPLNLSLSLQLFGRLTPKTEHIRYQAGRIKRWLVKQYQLVHLRELEIDSTRIQGYLIPHLLKTQSNELGSSVIKNWGMVSKYYLSLGYTLPPKDKFDFREVAPYWNLASQLREVTLESQKVDTRGKEKRKLYQVEDVEFEFKEGVVVIRAGLDGLLNEFLGGEKLGAVTYVEYLAFFKIINQRAQALLLTAICQALEPDLVPPCSGILSVYAEVDSVLRRAGQSAIDLLKLWEPLVLTKLGDVLGDRFGLEDDFKDTIRGEANKLAKKLHVTRSYKRMMKTLDQETRAQALFQYFGLFKHFAYPRVYSRETIEAIQEVSDKPSDSSPLNYLSDQCKIREEFYIRYTKAYHRAPAMNLGQLGQGSYLRQVLEAGKIPNAKNALYSLLEWFFVRFEKSIEWPLSDTLSTFLSDKAITQNRDIWYDGGSSGRDTTEKRLLLKFIKENEDSVEKVILKADEIYDKEADQIIALKVKEMELKIKGRGFGLMAFKPRLLQVLRESIAKKTSKLFPEITMTFSDLELKKKKFQLSRKSDDRRGYIHISKSLDINKFCTSQRQFNSLAVFQSLDELLGTDQLFTRVHEIFEKTWIVDGSASDPPDLVTFKARYEEALALGIEAPHVWADGAFSGLMGGIEGLCQYVWTICLLLRVERVLAVTQLTHFVMAQGDNVIINLIIPVEVDRVGGVVEGERARIQRISKDIDSALERELLRSGLTLKIEETLTSENLSIYGKDLHCPQHLTLAVKKAGSASIISSEQYQDVPTFLSGLGTGMETISECVNNKVSAHLFGVILGAAGWKSLAQRQTWKGWEYPFQNETSRRQVRSQGILLQKGESTMVHKEPEANPEKRTIELLLVSSLFGSALGMLPFPTPIDLEKRGVGDYVTHRLSIVKMALVSKKLPNRMIEMIVSTMNLPLSREQDLTKLFDSPFSLNLATEEDAASVIKRLARGTLRGLDIKNKKLADHIATMDQGITQIDEALASADTINPRIAYQFRNITDQKESEMFVTKFATAKTMRMVALSSSQDVSVVGLLNKRSQAKEIYTIWRTQRKGETLWTCSTQQAKKLRDRSWGKNIIGVTSPSPLEAILFKLIDPISWEEEKEAHHFTIHYYLSKPSLSSKTAHTTRGPLVPYFGTQTKPLIAKAYMELKGNPRTNKALQLLSMRETMIKAGSNLDKLLLSLCSNALDIDVNSLPSLQAQEEASAGEGVRGGIKESMSPVGPDNLYTHITHKVFERQWLSEFHVNIADFIIWGITKTRQHLQVNTDLGGSLPICVPACQECYREKERVFLDIPRETEWVNTSTTSDKAQTYFSTWCDLPRVSTLPSLDQKDATCLMGRSMATQKSTPGESITKFYSTAPDTHRLLHPVTLVLGYAEGTIFSYMKSQHNIHGSLYHPNIEEIEPALEKYVIDTKTSHTKHLGYLFQDADSLQELLETGMTPYIPRSIPLTITELTSACCMTLAKAISIVLRTGVTIPLMPENGYGENDIQVARLTANSLSRLMPRGRIQLVYLDCDLTSQMTAWVPTSQPSVLGSVNFHIEGVTISLTATEMRVGQESWEERKWTCSNNRHIIAKGVKTKSLFIHQSVPEIITHPPDLIVVIGGGLGGCVVPYLQKWRGPKVIFCTLFDERERISEDGDLIIPPELLVRGLAGRMIEKELLEAEMCDITVKGNRDLLIKVVQKWVQPNEHVLLIDEIENRGDQESVLQSSISELLTRMDSVCNLTSVHTIRETGPRQFAQRVNTIRRGRRTATLHWNQYNRRDQVEALLLVESHTRKTELHVTSSAVQAAFRKIDEKLESESRLEHSKWSLPELPPREKDILLGYVASVFLKLGLVVTDRHMSAAALITLLEEAGPKMISWDKKMEHRTWASSDAITEKGITQDQIFSLLCFAWALRGLKSGDWEHNADAIILQDVHIDTGPRLCQMGESPKRTFASFRLHNTKKAEDLKGYLGALLHLESFFPFGEQ